(19) United States
(12) Patent Application Publication
(10) Pub. No.: US 2026/0132471 A1
(43) Pub. Date: May 14, 2026

(54) GENOMIC BIOMARKERS OF EARLY RECURRENCE IN COLORECTAL CANCER (CRC)

(71) Applicant: Foundation Medicine, Inc., Boston, MA (US)

(72) Inventors: Richard Sheng Poe HUANG, Cary, NC (US); Julia Coelho Franca QUINTANILHA, Boston, MA (US)

(73) Assignee: Foundation Medicine, Inc., Boston, MA (US)

(21) Appl. No.: 19/386,725

(22) Filed: Nov. 12, 2025

Related U.S. Application Data
(63) Continuation of application No. PCT/US2024/029495, filed on May 15, 2024.
(60) Provisional application No. 63/466,969, filed on May 16, 2023.

(51) Int. Cl. C12Q 1/6886; G01N 33/57535

(52) U.S. Cl. CPC C12Q 1/6886; G01N 33/57535; C12Q 2600/118; C12Q 2600/156; G01N 2333/70532

(57) ABSTRACT

Provided herein are biomarkers associated with colorectal cancer (CRC) recurrence, as well as methods of treatment and uses related thereto. Detection of a biomarker of the disclosure can be used to identify individuals that may be at risk for CRC recurrence and/or may benefit from treatment with an anti-cancer therapy.

### CROSS-REFERENCE TO RELATED APPLICATION

This application is a continuation application of International Application No. PCT/US2024/029495, filed on May 15, 2024, which claims the priority benefit of U.S. Provisional Patent Application No. 63/466,969, filed on May 16, 2023, the disclosures of each of which are incorporated herein by reference in their entirety.

### TECHNICAL FIELD

Provided herein are biomarkers of colorectal cancer (CRC) recurrence, methods related to detecting such biomarkers, as well as methods of diagnosis/treatment and uses related thereto.

### BACKGROUND

Colorectal cancer (CRC) is a leading cause of death worldwide (Ogunwobi et al., Int J Mol Sci. (2020) 21(15): 5311). Despite an increasing trend in initial cure rates, 20-30% of patients with stage I-III CRC experience recurrence of disease (Qaderi et al., Int J Colorectal Dis (2021) 36:2399-2410).

Biomarkers such as microsatellite instability (MSI) and BRAF mutations have been used to help identify actionable targeted therapies for CRC patients (Bhalla et al. Clin Lab Med. (2018) 38:311-42). Prognostic biomarkers used in the clinic are typically based on the mutational status of genes known to be important in CRC carcinogenesis (NRAS, KRAS, BRAF) or associated with defects in the DNA mismatch repair system (MMR), the defects of which are the underlying mechanism of MSI (Popat et al., J. Clin. Oncol. (2004) 23:609-618. doi: 10.1200/JCO.2005.01.086).

The U.S. Food and Drug Administration (FDA) has cleared or approved a small number of diagnostics for CRC based on use of one or more of KRAS, EGFR, NRAS, and BRAF biomarkers (U.S. FDA, List of Cleared or Approved Companion Diagnostic Devices [In Vitro and Imaging Tools]; available at the website: www.fda.gov/medical-devices/vitro-diagnostics/list-cleared-or-approved-companion-diagnostic-devices-vitro-and-imaging-tools). However, to date, no biomarkers of recurrence of CRC have been identified. Thus, the genomics of CRC recurrence remain unclear.

Accordingly, there is a need in the art for characterizing biomarkers of CRC recurrence, and for developing methods, compositions, and assays for evaluating and treating patients with such biomarkers.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In some aspects, provided herein is a method of identifying an individual at risk for colorectal cancer (CRC) recurrence who may benefit from a treatment comprising an anti-cancer therapy, the method comprising detecting in one or more samples from the individual one or more biomarkers selected from: (a) a microsatellite instability high (MSI-H) status, (b) a high tumor mutational burden (TMB), (c) a PD-L1 positive status, and (d) an alteration in one or more genes, wherein the one or more genes comprise BRAF, PTEN, RNF43, ASXL1, CREBBP, MLL2, BCORL1, ATR, or SPEN, or any combination thereof; wherein detection of the one or more biomarkers in the one or more samples identifies the individual as being at risk for CRC recurrence and as likely to benefit from a treatment comprising an anti-cancer therapy.

In some aspects, provided herein is a method of identifying an individual at risk for CRC recurrence who may benefit from a treatment comprising an anti-cancer therapy, the method comprising detecting in one or more samples from the individual one or more biomarkers selected from: (a) an MSI-H status, (b) a high TMB, (c) a PD-L1 positive status, and (d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein detection of the one or more biomarkers in the one or more samples identifies the individual as being at risk for CRC recurrence and as likely to benefit from a treatment comprising an anti-cancer therapy.

In some aspects, provided herein is a method of identifying an individual at risk for CRC recurrence who may benefit from a treatment comprising an anti-cancer therapy, the method comprising detecting in one or more samples from the individual one or more biomarkers selected from: (a) an MSI-H status, (b) a high TMB, and (c) a PD-L1 positive status; wherein detection of the one or more biomarkers in the one or more samples identifies the individual as being at risk for CRC recurrence and as likely to benefit from a treatment comprising an anti-cancer therapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, (c) a PD-L1 positive status, and (d) an alteration in one or more genes, wherein the one or more genes comprise BRAF, PTEN, RNF43, ASXL1, CREBBP, MLL2, BCORL1, ATR, or SPEN, or any combination thereof, and wherein detection of the one or more biomarkers in the one or more samples identifies the individual as one who is at risk for CRC recurrence and may benefit from a treatment comprising an anti-cancer therapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, (c) a PD-L1 positive status, and (d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and wherein detection of the one or more biomarkers in the one or more samples identifies the individual as one who is at risk for CRC recurrence and may benefit from a treatment comprising an anti-cancer therapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, and (c) a PD-L1 positive status; and wherein detection of the one or more biomarkers in the one or more samples identifies the individual as one who is at risk for CRC recurrence and may benefit from a treatment comprising an anti-cancer therapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, (c) a PD-L1 positive status, and (d) an alteration in one or more genes, wherein the one or more genes comprise BRAF, PTEN, RNF43, ASXL1, CREBBP, MLL2, BCORL1, ATR, or SPEN, or any combination thereof, and wherein detection of the one or more biomarkers in the one or more samples identifies the individual as one who is at risk for CRC recurrence and may benefit from a chemotherapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, (c) a PD-L1 positive status, and (d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and wherein detection of the one or more biomarkers in the one or more samples identifies the individual as one who is at risk for CRC recurrence and may benefit from a chemotherapy.

In some aspects, provided herein is a method of selecting a therapy for an individual at risk for CRC recurrence, the method comprising detecting in one or more samples from the individual one or more biomarkers selected from: (a) an MSI-H status, (b) a high TMB, and (c) a PD-L1 positive status; and wherein detection of the one or more biomarkers in the one or more samples identifies the individual as one who is at risk for CRC recurrence and may benefit from a chemotherapy.

In some embodiments of any of the aspects provided herein, detection of the one or more biomarkers in the one or more samples identifies the individual as being at greater risk for CRC recurrence as compared to an individual with a CRC that does not comprise the one or more biomarkers.

In some aspects, provided herein is a method of identifying one or more treatment options for an individual at risk for CRC recurrence, the method comprising: (a) detecting in one or more samples from the individual one or more biomarkers selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof; and (b) generating a report comprising one or more treatment options identified for the individual based, at least in part, on detection of the one or more biomarkers in the one or more samples, wherein the one or more treatment options comprise an anti-cancer therapy, and wherein the report indicates that the individual is at risk for CRC recurrence based, at least in part, on detection of the one or more biomarkers in the one or more samples.

In some aspects, provided herein is a method of identifying one or more treatment options for an individual at risk for CRC recurrence, the method comprising: (a) detecting in one or more samples from the individual one or more biomarkers selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and (b) generating a report comprising one or more treatment options identified for the individual based, at least in part, on detection of the one or more biomarkers in the one or more samples, wherein the one or more treatment options comprise an anti-cancer therapy, and wherein the report indicates that the individual is at risk for CRC recurrence based, at least in part, on detection of the one or more biomarkers in the one or more samples.

In some aspects, provided herein is a method of identifying one or more treatment options for an individual at risk for CRC recurrence, the method comprising: (a) detecting in one or more samples from the individual one or more biomarkers selected from: (i) an MSI-H status, (ii) a high TMB, and (iii) a PD-L1 positive status; and (b) generating a report comprising one or more treatment options identified for the individual based, at least in part, on detection of the one or more biomarkers in the one or more samples, wherein the one or more treatment options comprise an anti-cancer therapy, and wherein the report indicates that the individual is at risk for CRC recurrence based, at least in part, on detection of the one or more biomarkers in the one or more samples.

In some aspects, provided herein is a method of identifying one or more treatment options for an individual at risk for CRC recurrence, the method comprising: (a) acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and (b) generating a report comprising one or more treatment options identified for the individual based at least in part on said knowledge, wherein the one or more treatment options comprise an anti-cancer therapy, and wherein the report indicates that the individual is at risk for CRC recurrence based, at least in part, on the knowledge of the one or more biomarkers in the one or more samples.

In some aspects, provided herein is a method of identifying one or more treatment options for an individual at risk for CRC recurrence, the method comprising: (a) acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and (b) generating a report comprising one or more treatment options identified for the individual based at least in part on said knowledge, wherein the one or more treatment options comprise an anti-cancer therapy, and wherein the report indicates that the individual is at risk for CRC recurrence based, at least in part, on the knowledge of the one or more biomarkers in the one or more samples.

In some aspects, provided herein is a method of identifying one or more treatment options for an individual at risk for CRC recurrence, the method comprising: (a) acquiring knowledge of one or more biomarkers in one or more samples from the individual, wherein the one or more biomarkers are selected from: (i) an MSI-H status, (ii) a high TMB, and (iii) a PD-L1 positive status; and (b) generating a report comprising one or more treatment options identified for the individual based at least in part on said knowledge, wherein the one or more treatment options comprise an anti-cancer therapy, and wherein the report indicates that the individual is at risk for CRC recurrence based, at least in part, on the knowledge of the one or more biomarkers in the one or more samples.

In some embodiments of any of the aspects provided herein, the report indicates that the individual is at greater risk for CRC recurrence as compared to an individual with a CRC that does not comprise the one or more biomarkers.

In some aspects, provided herein is a method of selecting a treatment for an individual at risk for CRC recurrence, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof; and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

In some aspects, provided herein is a method of selecting a treatment for an individual at risk for CRC recurrence, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

In some aspects, provided herein is a method of selecting a treatment for an individual at risk for CRC recurrence, comprising acquiring knowledge of one or more biomarkers in one or more samples from the individual, wherein the one or more biomarkers are selected from: (i) an MSI-H status, (ii) a high TMB, and (iii) a PD-L1 positive status; and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

In some embodiments of any of the aspects provided herein, responsive to the acquisition of said knowledge, the individual is classified as being at greater risk for CRC recurrence as compared to an individual with a CRC that does not comprise the one or more biomarkers.

In some aspects, provided herein is a method of predicting survival of an individual having a CRC, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof; and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting survival of an individual having a CRC, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting survival of an individual having a CRC, comprising acquiring knowledge of one or more biomarkers in one or more samples from the individual, wherein the one or more biomarkers are selected from: (i) an MSI-H status, (ii) a high TMB, and (iii) a PD-L1 positive status; and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting survival of an individual having a CRC treated with a treatment comprising an anti-cancer therapy, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting survival of an individual having a CRC treated with a treatment comprising an anti-cancer therapy, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting survival of an individual having a CRC treated with a treatment comprising an anti-cancer therapy, comprising acquiring knowledge of one or more biomarkers in one or more samples from the individual, wherein the one or more biomarkers are selected from: (i) an MSI-H status, (ii) a high TMB, and (iii) a PD-L1 positive status; and wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

In some aspects, provided herein is a method of predicting likelihood of recurrence of a CRC in an individual, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof; and wherein responsive to the acquisition of said knowledge, the individual is predicted to be at risk of CRC recurrence.

In some aspects, provided herein is a method of predicting likelihood of recurrence of a CRC in an individual, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein responsive to the acquisition of said knowledge, the individual is predicted to be at risk of CRC recurrence.

In some aspects, provided herein is a method of predicting likelihood of recurrence of a CRC in an individual, comprising acquiring knowledge of one or more biomarkers in one or more samples from the individual, wherein the one or more biomarkers are selected from: (i) an MSI-H status, (ii) a high TMB, and (iii) a PD-L1 positive status; and wherein responsive to the acquisition of said knowledge, the individual is predicted to be at risk of CRC recurrence.

In some embodiments of any of the aspects provided herein, the individual is predicted to be at greater risk for CRC recurrence as compared to an individual with a CRC that does not comprise the one or more biomarkers.

In some aspects, provided herein is a method of treating or delaying progression of a CRC, comprising: (a) detecting one or more biomarkers in one or more samples from an individual having a CRC, wherein the one or more biomarkers comprise an MSI-H status, wherein responsive to detecting the one or more biomarkers in the one or more samples, the individual is identified as being at risk for CRC recurrence; and (b) responsive to detecting the one or more biomarkers in the one or more samples, administering to the individual an effective amount of a treatment that comprises an anti-cancer therapy. In some embodiments, the one or more biomarkers further comprise one or more of: a high TMB; a PD-L1 positive status; and an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof. In some embodiments, the one or more biomarkers comprise an alteration in one or more genes, wherein the one or more genes comprise BRAF, PTEN, RNF43, ASXL1, CREBBP, MLL2, BCORL1, ATR, or SPEN, or any combination thereof. In some embodiments, the anti-cancer therapy comprises an immunotherapy.

In some aspects, provided herein is a method of treating or delaying progression of a CRC, comprising: (a) detecting two or more biomarkers in one or more samples from an individual having a CRC, wherein the two or more biomarkers comprise: (i) an MSI-H status and one or more of: (ii) a high TMB, and (iii) a PD-L1 positive status, wherein responsive to detecting the one or more biomarkers in the one or more samples, the individual is identified as being at risk for CRC recurrence; and (b) responsive to detecting the one or more biomarkers in the one or more samples, administering to the individual an effective amount of a treatment that comprises an anti-cancer therapy. In some embodiments, the one or more biomarkers further comprise an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof. In some embodiments, the one or more biomarkers comprise an alteration in one or more genes, wherein the one or more genes comprise BRAF, PTEN, RNF43, ASXL1, CREBBP, MLL2, BCORL1, ATR, or SPEN, or any combination thereof. In some embodiments, the anti-cancer therapy comprises an immunotherapy.

In some aspects, provided herein is a method of treating or delaying progression of a CRC in an individual, comprising: (a) acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, wherein responsive to the acquisition of said knowledge the individual is identified as being at risk for CRC recurrence; and (b) responsive to said knowledge, administering to the individual an effective amount of a treatment that comprises an anti-cancer therapy.

In some aspects, provided herein is a method of treating or delaying progression of a CRC in an individual, comprising: (a) acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein responsive to the acquisition of said knowledge the individual is identified as being at risk for CRC recurrence; and (b) responsive to said knowledge, administering to the individual an effective amount of a treatment that comprises an anti-cancer therapy.

In some aspects, provided herein is a method of treating or delaying progression of a CRC in an individual, comprising: (a) acquiring knowledge of one or more biomarkers in one or more samples from the individual, wherein the one or more biomarkers are selected from: (i) an MSI-H status, (ii) a high TMB, and (iii) a PD-L1 positive status, wherein responsive to the acquisition of said knowledge the individual is identified as being at risk for CRC recurrence; and (b) responsive to said knowledge, administering to the individual an effective amount of a treatment that comprises an anti-cancer therapy.

In some aspects, provided herein is a method of treating or delaying progression of a CRC in an individual, comprising administering to an individual having a CRC an effective amount of a treatment that comprises an anti-cancer therapy, wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof.

In some aspects, provided herein is a method of treating or delaying progression of a CRC in an individual, comprising administering to an individual having a CRC an effective amount of a treatment that comprises an anti-cancer therapy, wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof.

In some aspects, provided herein is a method of treating or delaying progression of a CRC in an individual, comprising administering to an individual having a CRC an effective amount of a treatment that comprises an anti-cancer therapy, wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from: (i) an MSI-H status, (ii) a high TMB, and (iii) a PD-L1 positive status.

In some embodiments of any of the aspects provided herein, wherein the individual is identified as being at greater risk for CRC recurrence as compared to an individual with a CRC that does not comprise the one or more biomarkers.

In some aspects, provided herein is a method of monitoring, evaluating or screening an individual for CRC recurrence risk, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and wherein responsive to the acquisition of said knowledge, the individual is predicted to have increased risk of CRC recurrence, as compared to an individual with a CRC that does not comprise the one or more biomarkers.

In some aspects, provided herein is a method of monitoring, evaluating or screening an individual for CRC recurrence risk, comprising acquiring knowledge of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein responsive to the acquisition of said knowledge, the individual is predicted to have increased risk of CRC recurrence, as compared to an individual with a CRC that does not comprise the one or more biomarkers.

In some aspects, provided herein is a method of monitoring, evaluating or screening an individual for CRC recurrence risk, comprising acquiring knowledge of one or more biomarkers in one or more samples from the individual, wherein the one or more biomarkers are selected from: (i) an MSI-H status, (ii) a high TMB, and (iii) a PD-L1 positive status; and wherein responsive to the acquisition of said knowledge, the individual is predicted to have increased risk of CRC recurrence, as compared to an individual with a CRC that does not comprise the one or more biomarkers.

In some aspects, provided herein is a method of assessing one or more biomarkers of CRC recurrence, the method comprising: (a) detecting one or more biomarkers in one or more samples from an individual having a CRC, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and (b) providing an assessment of the one or more biomarkers in the one or more samples, wherein detecting the one or more biomarkers in the one or more samples from the individual identifies the individual as being at risk for CRC recurrence.

In some aspects, provided herein is a method of assessing one or more biomarkers of CRC recurrence, the method comprising: (a) detecting one or more biomarkers in one or more samples from an individual having a CRC, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and (b) providing an assessment of the one or more biomarkers in the one or more samples, wherein detecting the one or more biomarkers in the one or more samples from the individual identifies the individual as being at risk for CRC recurrence.

In some aspects, provided herein is a method of assessing one or more biomarkers of CRC recurrence, the method comprising: (a) detecting one or more biomarkers in one or more samples from an individual having a CRC, wherein the one or more biomarkers are selected from: (i) an MSI-H status, (ii) a high TMB, and (iii) a PD-L1 positive status; and (b) providing an assessment of the one or more biomarkers in the one or more samples, wherein detecting the one or more biomarkers in the one or more samples from the individual identifies the individual as being at risk for CRC recurrence.

In some aspects, provided herein is a method of detecting the presence or absence of a CRC in an individual, the method comprising: (a) detecting the presence or absence of a CRC in a sample from the individual; and (b) detecting the presence or absence of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, wherein detecting the presence of a CRC and the one or more biomarkers in one or more samples from the individual identifies the individual as being at risk for CRC recurrence.

In some aspects, provided herein is a method of detecting the presence or absence of a CRC in an individual, the method comprising: (a) detecting the presence or absence of a CRC in a sample from the individual; and (b) detecting the presence or absence of one or more biomarkers in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein detecting the presence of a CRC and the one or more biomarkers in one or more samples from the individual identifies the individual as being at risk for CRC recurrence.

In some aspects, provided herein is a method of detecting the presence or absence of a CRC in an individual, the method comprising: (a) detecting the presence or absence of a CRC in a sample from the individual; and (b) detecting the presence or absence of one or more biomarkers in one or more samples from the individual, wherein the one or more biomarkers are selected from: (i) an MSI-H status, (ii) a high TMB, and (iii) a PD-L1 positive status, wherein detecting the presence of a CRC and the one or more biomarkers in one or more samples from the individual identifies the individual as being at risk for CRC recurrence.

In some aspects, provided herein is a method for monitoring recurrence of a CRC in an individual, the method comprising: (a) detecting, in one or more samples obtained from the individual at a first time point, the presence or absence of one or more biomarkers; (b) detecting, in one or more samples obtained from the individual at a second time point after the first time point, the presence or absence of one or more biomarkers; and (c) providing an assessment of CRC recurrence risk in the individual based, at least in part, on the presence or absence of the one or more biomarkers in the one or more samples at the first time point and/or at the second time point; wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, wherein detecting the presence of the one or more biomarkers in the one or more samples at the first time point and/or at the second time point identifies the individual as being at risk for CRC recurrence.

In some aspects, provided herein is a method for monitoring recurrence of a CRC in an individual, the method comprising: (a) detecting, in one or more samples obtained from the individual at a first time point, the presence or absence of one or more biomarkers; (b) detecting, in one or more samples obtained from the individual at a second time point after the first time point, the presence or absence of one or more biomarkers; and (c) providing an assessment of CRC recurrence risk in the individual based, at least in part, on the presence or absence of the one or more biomarkers in the one or more samples at the first time point and/or at the second time point; wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein detecting the presence of the one or more biomarkers in the one or more samples at the first time point and/or at the second time point identifies the individual as being at risk for CRC recurrence.

In some aspects, provided herein is a method for monitoring recurrence of a CRC in an individual, the method comprising: (a) detecting, in one or more samples obtained from the individual at a first time point, the presence or absence of one or more biomarkers; (b) detecting, in one or more samples obtained from the individual at a second time point after the first time point, the presence or absence of one or more biomarkers; and (c) providing an assessment of CRC recurrence risk in the individual based, at least in part, on the presence or absence of the one or more biomarkers in the one or more samples at the first time point and/or at the second time point; wherein the one or more biomarkers are selected from: (i) an MSI-H status, (ii) a high TMB, and (iii) a PD-L1 positive status, wherein detecting the presence of the one or more biomarkers in the one or more samples at the first time point and/or at the second time point identifies the individual as being at risk for CRC recurrence.

In some embodiments of any of the aspects provided herein, the methods further comprise selecting a treatment, administering a treatment, adjusting a treatment, adjusting a dose of a treatment, or applying a treatment to the individual based, at least in part, on detecting the presence of the one or more biomarkers at the first time point and/or at the second time point, wherein the treatment comprises an anti-cancer therapy.

In some aspects, provided herein is a method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, or SPEN, or any combination thereof, wherein identifying the presence of the one or more biomarkers in the one or more samples identifies the individual as being at risk for CRC recurrence, wherein the candidate treatment comprises an anti-cancer therapy.

In some aspects, provided herein is a method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein identifying the presence of the one or more biomarkers in the one or more samples identifies the individual as being at risk for CRC recurrence, wherein the candidate treatment comprises an anti-cancer therapy.

In some aspects, provided herein is a method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from: (i) an MSI-H status, and (ii) a high TMB, wherein identifying the presence of the one or more biomarkers in the one or more samples identifies the individual as being at risk for CRC recurrence, wherein the candidate treatment comprises an anti-cancer therapy.

In some embodiments of any of the aspects provided herein, the methods further comprise detecting the presence or absence of an additional biomarker in sample from the individual. In some embodiments, the additional biomarker is a PD-L1 positive status. In some embodiments, the presence of the additional biomarker in the sample identifies the individual as being at risk for CRC recurrence. In some embodiments, the presence of the biomarker(s) in one or more samples from the individual identifies the individual as one who may benefit from a treatment comprising an anti-cancer therapy. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS).

In some aspects, provided herein is a method of treating or delaying progression of a CRC, comprising: (a) detecting one or more biomarkers in one or more samples from an individual having a CRC, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, wherein responsive to detecting the one or more biomarkers in the one or more samples, the individual is identified as being at risk for CRC recurrence; and (b) responsive to detecting the one or more biomarkers in the one or more samples, administering to the individual an effective amount of a treatment that comprises an anti-cancer therapy.

In some aspects, provided herein is a method of treating or delaying progression of a CRC, comprising: (a) detecting one or more biomarkers in one or more samples from an individual having a CRC, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, wherein responsive to detecting the one or more biomarkers in the one or more samples, the individual is identified as being at risk for CRC recurrence; and (b) responsive to detecting the one or more biomarkers in the one or more samples, administering to the individual an effective amount of a treatment that comprises an anti-cancer therapy.

In some aspects, provided herein is a method of treating or delaying progression of a CRC, comprising: (a) detecting one or more biomarkers in one or more samples from an individual having a CRC, wherein the one or more biomarkers are selected from: (i) an MSI-H status, (ii) a high TMB, and (iii) a PD-L1 positive status, wherein responsive to detecting the one or more biomarkers in the one or more samples, the individual is identified as being at risk for CRC recurrence; and (b) responsive to detecting the one or more biomarkers in the one or more samples, administering to the individual an effective amount of a treatment that comprises an anti-cancer therapy.

In some embodiments of any of the aspects or embodiments provided herein, the individual is identified as being at greater risk for CRC recurrence as compared to an individual with a CRC that does not comprise the one or more biomarkers. In some embodiments, the acquiring knowledge of the one or more biomarkers in one or more samples from the individual comprises detecting the one or more biomarkers in the one or more samples.

In some embodiments of any of the aspects or embodiments provided herein, the CRC is early CRC.

In some embodiments of any of the aspects or embodiments provided herein, the CRC is Stage 0, Stage I, Stage II, Stage III, or Stage IV CRC. In some embodiments, the CRC is a Stage IV CRC. In some embodiments, the CRC comprises an alteration in an APC and/or TP53 gene; optionally wherein: (a) the alteration in an APC gene comprises one or more of: R1450*, R876*, splice site 835−8A>G, T1556fs*3, E1309fs*4, R213*, R216*, R564*, R283*, R232*, R1114*, S1465fs*3, Q1367*, R805*, R499*, Q1429*, Q1406*, E1379*, Q1338*, E1309*, E1306*, Y935*, R554*, E941*, Q1378*, V1414fs*1, R302*, E1353*, Q1291*, Y935fs*1, S1356*, E1322*, E1408*, Q1303*, E1397*, P1319fs*2, E1464fs*8, E1295*, E1286*, L1488fs*19, Q1294*, N1455fs*18, S1411fs*4, K534*, G1312*, S1495fs*12, S1344*, R1399fs*9, F1491fs*16, S1415fs*4, S1400*, S1346*, S1315*, Q1328*, E1345*, E1317*, T1493fs*14, S1436fs*37, S1421fs*52, K1182*, D1486fs*21, R332*, F1396fs*19, A1492fs*22, splice site 1548+1G>A, S457*, S1545*, S1465fs*9, R640W, S943*, S1501fs*6, Q188*, Q1477*, Q1244*, P1442fs*31, P1440fs*33, P1439fs*34, K670*, I1580fs*70, E763*, E1554*, W699*, W423*, V1452fs*21, S1415fs*8, S1355fs*19, Q767*, Q236*, L1302fs*3, E893*, E1573*, E1544*, Y1376*, T1556fs*9, T1438fs*35, Q1480*, Q1096*, P1443fs*30, P1424fs*49, M1383fs*3, L1488fs*26, H1490fs*24, E225*, E1538fs*5, E1374*, E1353fs*62, E1309fs*6, C1387*, Y1376fs*9, W1049*, S932*, S811*, S770*, S1539*, S1400fs*1, S1389fs*5, S1355fs*60, S1327*, S1200fs*7, R653K, R1314fs*7, Q901*, Q793*, Q695*, Q264*, Q1228*, Q1065*, P1433fs*40, L1489fs*19, K1310fs*11, I1580fs*69, I1311fs*4, G1288*, F814fs*6, E991*, E403fs*51, E1577fs*73, E1552*, E1536*, E1530*, E1461*, E1353fs*21, E1284*, E1155*, D170fs*4, C1410*, A1351fs*3, Y935fs*19, Y796fs*2, Y1376fs*10, Y1075*, W685*, V452fs*7, T1445fs*28, splice site 835−1G>A, splice site 645+1G>A, splice site 1409−5A>G, splice site 1409−2A>G, splice site 1312+1G>A, S596*, S1581fs*69, S1567*, S1495fs*19, S1355fs*20, S1298fs*7, S1282*, S1272*, R904fs*7, R904fs*12, R405*, R2237*, Q886*, Q789*, Q757*, Q445*, Q260*, Q1477fs*30, Q1469*, Q1444*, or Q1256*; and/or (b) the alteration in a TP53 gene comprises one or more of: R175H, R282W, R273H, R248Q, R273C, G245S, R213*, R196*, R248W, R306*, R342*, C176F, M237I, C238Y, P152L, Y220C, G266E, C141Y, splice site 375G>A, V173L, V173M, C135F, R158H, W146*, C176Y, I195T, C275Y, G244S, H179R, H179Y, H214R, R249S, R337C, T211I, V172F, V272M, E285K, E286K, P151S, P278S, R273L, splice site 673−1G>A, T125M, V272L, C275F, E294*, F113V, G244D, G245D, K132N, P27fs*17, Q104*, splice site 672+1G>T, splice site 782+1G>A, V122fs*26, Y163C, A161T, C135Y, C242F, G266R, L257P, L257Q, N131fs*27, P151H, P153fs*28, S166*, S215G, S215N, splice site 375+1G>T, splice site 559+1G>A, V216M, V73fs*76, C238F, C242fs*5, C242Y, E171*, E204*, E258G, E271K, G244A, G266*, H178fs*3, H193Y, K132R, L194F, N239fs*9, P250L, P278T, Q165*, R110L, R209fs*6, R213L, R213Q, S127F, S215I, splice site 672G>A, splice site 782+1G>T, splice site 88_96+1delAACGTTCTGG, V274F, Y126C, Y126N, Y205D, Y234C, Y236C, A159V, A276G, C135R, C135W, C141R, C141W, C277F, D259Y, E198*, E258*, E271*, E298*, E51*, F134L, F270C, G244C, G266V, H193L, L130F, L130V, L194P, L35fs*9, MIT, N247L, P191del, Q167*, Q192*, Q317*, R181H, R181P, R249M, S214W, S241fs*6, S261fs*85, S90fs*33, S90fs*59, splice site 375+5G>T, splice site 375G>C, splice site 376−1G>A, splice site 560−1G>A, splice site 560−1G>T, splice site 560−2A>T, splice site 560−3T>G, splice site 673−1G>T, splice site 920−2A>G, splice site 993+1G>A, V157F, V216L, V274A, V274G, W91*, Y163H, Y205H, Y234H, Y236fs*14, Y236H, C124fs*25, C141*, C182fs*65, C229*, C238R, D393fs*78, E180*, E198fs*49, E221*, E224D, E258A, E258K, E285*, E336fs*4, E339*, E349*, F109V, F134C, F134V, or F270L. In some embodiments, the CRC is Stage I, Stage II, or Stage III CRC. In some embodiments, the CRC comprises an alteration in one or more of a BRCA1, BRCA2, MSH6, MLH1, and/or MSH2 gene, optionally wherein: (a) the alteration in a BRCA1 gene comprises one or more of: K339fs*2, K654fs*47, Q1756fs*74, Y655fs*18, A224fs*4, E181*, E577*, E732*, H1686R, K1711fs*3, L1098fs*4, L63F, Q74*, R1203*, R1495M, R1751*, S1457*, S324fs*16, splice site 4185+2_4185+22>A, splice site 442−2A>G, splice site 5277+1G>A, or V340fs*6; (b) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*; (c) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H; (d) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G; and/or (e) the alteration in a MSH2 gene comprises one or more of: A230fs*16, S233fs*13, R680*, E580*, E480*, Y408*, splice site 943−1G>C, splice site 942+3A>T, R406*, R389*, Q61*, Q574*, Q324*, L634*, L277fs*5, L187R, K449fs*5, I134fs*8, G683R, E86fs*4, E850*, E188*, C778fs*35, or C778fs*34. In some embodiments, the CRC comprises an alteration in one or more of a RNF43, MLL2, MSH3, PTCH1, CDK12, ARID1A, ASXL1, MSH6, BCORL1, CTNNB1, MLH1, CIC, MAP3K, ATR, MSH2, CTCF, JAK1, QKI, CDH1, CASP8, NOTCH3, EP300, BRCA2, MEN1, or BCOR gene, or any combination thereof, optionally wherein: (a) the alteration in a RNF43 gene comprises one or more of: G659fs*41, R117fs*41, R225fs*194, R117fs*8, R145*, P660fs*41, P660fs*87, R371*, R132*, R330*, R337*, A273fs*147, E37fs*11, K181fs*4, S216L, V479fs*25, Y248*, Y332*, A169T, A193fs*6, A78T, C290*, E258fs*162, E318*, E37*, F103fs*20, G257fs*162, G29*, G659fs*87, H352fs*87, I48T, K60fs*2, L311fs*108, L311fs*132, L53fs*1, L82*, L88fs*13, M1I, M55fs*7, N167fs*1, P370fs*49, P715fs*15, P77fs*18, Q153*, Q233*, Q254*, Q283*, Q426*, Q426fs*77, Q6fs*29, Q8*, R113*, R225fs*195, R286W, R49fs*3, S607L, S687fs*13, splice site 375+1G>A, splice site 583−177_592del187, splice site 687+1G>A, splice site 688−1G>A, splice site 850−2A>G, splice site 952+2T>C, T158fs*10, V271fs*11, V271fs*149, V299fs*120, V490fs*12, W13*, W13fs*26, W159*, W165*, W200*, W302*, Y332fs*110, or Y332fs*111; (b) the alteration in a MLL2 gene comprises one or more of: P2354fs*30, G1235fs*95, P647fs*283, P648fs*2, R4904*, A1390fs*27, Q836fs*94, A2119fs*25, C2436fs*49, C346fs*17, G5182fs*61, H1497fs*30, R2443fs*6, R4238C, R845fs*3, T382fs*20, T4629fs*11, V1244fs*86, V4799M, A1390fs*42, A2169T, A2205fs*59, A221fs*40, A3552fs*4, C5123*, C5142fs*5, D2769N, E2962fs*42, F1790fs*12, F2494fs*49, F2566fs*17, G1317*, G1960fs*87, G1995*, G2262fs*37, G2265fs*21, G3189*, G3698fs*51, H77fs*53, I4491fs*1, I977fs*23, K1686fs*36, K304fs*30, K3140fs*2, K4843fs*15, L1020fs*36, L1271fs*15, L2331fs*46, L2594fs*97, L3716fs*296, L3880fs*131, L5183fs*16, L5318fs*14, P1460fs*46, P2206fs*58, P2382fs*2, P367fs*35, P4380fs*4, P444fs*2, P4968fs*27, P506fs*424, P583fs*347, P62fs*9, P886fs*44, Q1377R, Q1557*, Q211*, Q3471*, Q3811fs*201, Q3839fs*42, Q3909fs*103, Q3934*, Q3950R, Q4235fs*98, Q4284*, Q791fs*139, Q809fs*121, Q809fs*3, R1252*, R1687fs*4, R2099*, R2471*, R2771*, R2830*, R4198*, R466C, R5048H, R5086*, R5120C, R5282*, R5454*, R755fs*3, S102fs*28, S1107fs*12, S1684fs*38, S1684T, S2532fs*11, S2910fs*32, S3159fs*16, S4010fs*12, S4507fs*12, S456*, S4789fs*27, splice site 14644−1G>T, splice site 16413−2A>G, T209fs*11, T2191fs*11, T698fs*232, V1670fs*52, V3089fs*30, Y2199fs*65, or Y2907fs*3; (c) the alteration in a MSH3 gene comprises one or more of: K383fs*32, L564fs*1, E342*, K902fs*5, K99fs*3, N1020fs*17, N385fs*19, N524fs*3, N739fs*8, N861fs*6, or R268*; (d) the alteration in a PTCH1 gene comprises one or more of: S1203fs*52, R1308fs*64, Y1316fs*56, L39fs*41, R1308fs*17, E61fs*18, N97fs*43, V1164I, A563V, C1398fs*54, C727fs*19, E1242K, G526*, L50fs*39, N97fs*20, P643fs*11, R602*, or R6fs*1; (e) the alteration in a CDK12 gene comprises one or more of: G1461fs*38, Q1291fs*3, T1463fs*50, G1271fs*23, R983*, E59fs*33, E751*, E887*, H1035fs*7, I873fs*11, K445*, L342fs*8, L996*, N474fs*8, N864fs*2, P683fs*70, P686fs*13, P974L, Q115*, Q1418*, R1048*, R1331*, R298*, R890H, S133fs*24, splice site 1047−2A>C, T1346fs*7, or T212fs*18; (f) the alteration in a ARID1A gene comprises one or more of: D1850fs*33, D1850fs*4, F2141fs*59, G276fs*87, P1326fs*155, P224fs*8, Q766fs*67, K1072fs*21, Q1452fs*29, Q372fs*19, Q758fs*75, Q802fs*15, R1989*, A339fs*24, M1634fs*14, Q372fs*28, R693*, Y551fs*72, G314fs*49, P1115fs*46, P1568fs*44, P1898fs*25, Q1200*, Q1519fs*8, Q1631*, R1150fs*4, S1000Y, S11fs*91, W1073fs*32, A134fs*98, A1539fs*27, A27fs*24, A339fs*61, A62fs*39, A77fs*24, D1850fs*34, D2178fs*47, D2260fs*5, E1297*, E1733*, E1783fs*6, E2058*, E2120*, E992*, G1110fs*51, G122fs*278, G126fs*274, G1740*, G2069fs*50, G2087R, G236fs*163, G277fs*86, G285fs*78, G37fs*14, G801fs*32, G82fs*19, G83fs*28, G987fs*50, K1094fs*67, K1905fs*18, K250*, L1049fs*55, L1841fs*2, L2082fs*53, L2238fs*30, L2270fs*8, M1154fs*7, M1273fs*10, M1318fs*163, M1388fs*94, M1564fs*8, M1595fs*19, M1634fs*1, M890fs*46, N1313fs*168, N2109fs*26, P1175fs*5, P1451fs*41, P1468fs*13, P146fs*86, P1560fs*5, P225fs*175, P469fs*150, Q1188*, Q1212*, Q1250*, Q1327*, Q1327fs*11, Q1420*, Q1512*, Q1584*, Q1650*, Q1708*, Q1835*, Q1835fs*1, Q1974*, Q2115*, Q2176*, Q2176fs*48, Q288*, Q505fs*117, Q521*, Q538*, Q546fs*73, Q566*, Q575fs*46, Q581*, Q611*, Q633*, Q806fs*11, R1223C, R1335*, R1446*, R1461*, R1658fs*40, R1658W, R1722*, R1869fs*30, S1465fs*25, S1645fs*46, S2264*, S255fs*145, S366fs*25, S536fs*87, S617fs*2, S617fs*6, splice site 1921−3_1925delTAGGATCT, splice site 2879−2A>G, splice site 3715+1G>C, splice site 4005−2A>T, T1514fs*13, T1743M, T2252fs*27, T286fs*114, T894fs*25, V1561fs*11, V63fs*38, W1545*, W1670*, W2050*, W2091*, Y1101fs*1, Y1377*, Y2076*, Y222*, Y422*, or Y551fs*68; (g) the alteration in a ASXL1 gene comprises one or more of: G645fs*58, G646fs*12, R693*, E635fs*15, G646fs*58, Q592*, A627fs*8, E41K, E518*, E566*, E676*, E917*, F354L, G1376fs*74, G643fs*15, G643fs*61, G967del, L983fs*8, N1158fs*6, P1377fs*3, P763fs*12, P808fs*10, Q561fs*1, Q588*, Q695*, Q768fs*6, R541fs*162, R596fs*107, R718fs*7, S1335fs*115, S747fs*25, S892fs*16, splice site 140+2T>G, splice site 471+1G>A, T957fs*26, V737fs*10, W1037*, or W583*; (h) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H; (i) the alteration in a BCORL1 gene comprises one or more of: P1681fs*20, A1166fs*56, G1682fs*4, A74fs*42, M644fs*4, A858fs*67, Q1001fs*49, R1299*, S803fs*83, A74fs*20, A971fs*4, E1655*, E619*, I389fs*29, K1207N, K1330fs*17, L275fs*143, N1412fs*38, P323fs*95, Q459*, R1196*, R1297*, R1338*, R1420*, R609*, R743fs*13, splice site 4306−2A>G, or W1105*; (j) the alteration in a CTNNB1 gene comprises one or more of: S45F, T41A, S45P, Q773*, R587*, S33C, T41I, D32N, E334K, G34E, N387K, R449C, S45A, T257L, W25*, W383R, D17_Q78del, D17_T75del, E568*, G69*, I35_G38del, I35S, K19_S37>N, K335fs*10, K335L, M8_V79del, R376H, R515Q, R582Q, R582W, R90*, R95*, S33F, S33T, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242−39del684, splice site 14−338_81>TTAC, splice site 14−39_225del251, splice site 14−48_241+12del288, splice site 14−5_97del89, splice site 14−6_241+74del308, splice site 14−69_242-10del488, splice site 14−7_89del83, splice site 14−80_241+22>CAT, splice site 14−91_241+25del344, splice site 1954+1G>A, splice site 1954+1G>T, splice site 1955−1G>A, splice site 2138−2A>C, splice site 241+1G>C, splice site 32_241+94del304, splice site 60_287del428, splice site 65_274del410, splice site 74_241+52del220, splice site 78_241+11del175, splice site 87_242−79del277, splice site 98_241+8>A, V22_S33del, V22_S37del, W25_I35>C, W383C, or W776*; (k) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G; (l) the alteration in a CIC gene comprises one or more of: P1597fs*23, P1248fs*54, P509fs*14, P1116fs*45, T1375fs*40, P135fs*70, P1598fs*16, S1117fs*34, S961fs*6, A785fs*139, A900fs*24, D449fs*23, E367*, G136fs*8, G1600fs*14, P1128fs*33, P1336fs*3, P1529fs*91, P404fs*31, P515fs*8, P518fs*5, P574fs*154, P768fs*156, P911fs*13, P98fs*107, Q378*, R1313W, R201W, R353*, S902fs*21, S904fs*27, splice site 583−1G>A, T1541fs*18, T1541fs*79, or T328fs*78; (m) the alteration in a MAP3K1 gene comprises one or more of: C635*, E126*, E1293fs*3, E788*, G1074fs*8, G608fs*48, H114fs*50, K1160fs*12, L380fs*4, L915*, N1212fs*33, P74fs*3, Q1022*, Q320*, R208*, R288*, R307fs*5, R532*, S101fs*63, splice site 3983−1G>A, splice site 483−1G>A, splice site 633+2T>A, T1145fs*6, T457fs*31, V1045fs*12, or V569I; (n) the alteration in a ATR gene comprises one or more of: I774fs*5, R1814fs*10, F1091fs*28, F1134fs*6, I774fs*3, E148*, E1699*, E2579*, F2168*, F222fs*11, I1264fs*14, I691fs*5, I774fs*6, K446fs*11, K773fs*3, L1029fs*20, Q195*, R1015Q, R1814fs*8, R2001*, R223fs*1, R224fs*18, R2533*, R2547*, R2598*, S2207fs*15, S825fs*13, splice site 5381−1G>A, W1591*, or Y1844*; (o) the alteration in a MSH2 gene comprises one or more of: A230fs*16, C778fs*34, E188*, E86fs*4, R680*, S233fs*13, or Y408*; (p) the alteration in a CTCF gene comprises one or more of: T204fs*26, T204fs*18, R166C, E363fs*5, A137fs*17, A225V, D194fs*28, E112*, E145*, E182fs*9, G32fs*30, H19fs*15, MiT, N259fs*44, P50L, R11W, R275C, splice site 374−1G>T, splice site 854−1G>T, T317fs*91, Y15H, or Y195*; (q) the alteration in a JAK1 gene comprises one or more of: K860fs*16, P430fs*2, G741D, or K496N; (r) the alteration in a QKI gene comprises one or more of: K134fs*14, A313V, E42*, G77fs*14, L236fs*54, R319*, splice site 1010−233_*194del444, splice site 142+1G>A, splice site 143−1G>T, or splice site 546+1G>T; (s) the alteration in a CDH1 gene comprises one or more of: P126fs*89, P127fs*41, R492fs*44, S70fs*13, A241fs*3, A634V, C28*, D257G, D291N, D400G, G169fs*46, L214P, L711V, P372fs*8, Q16*, R335*, R63*, R74*, S111fs*6, S18fs*39, splice site 1137G>A, splice site 1138−1G>A, splice site 1565+1G>A, splice site 1711+2T>C, splice site 688−1G>T, T323fs*33, T340M, or W532*; (t) the alteration in a CASP8 gene comprises one or more of: R449*, K490fs*73, F373fs*26, I350fs*4, E212*, R452*, A197fs*14, P411L, R194fs*17, R68*, Y252*, A264fs*24, C196Y, D380fs*19, D380fs*2, E195*, E36*, F152fs*18, F296fs*11, G11R, K478fs*10, K478fs*19, L59fs*12, L62P, N475fs*13, R250W, splice site 1355+2T>C, splice site 151+1G>A, V222fs*13, V268fs*8, V492fs*71, or Y8fs*1; (u) the alteration in a NOTCH3 gene comprises one or more of: A1802fs*8, C1344fs*76, P695fs*165, C43fs*32, G1318fs*245, G2035fs*50, P1317fs*103, T250fs*122, A1020fs*252, A1927T, C720fs*1, C87fs*149, D352fs*2, E1492fs*84, G2035fs*60, G2081fs*4, G707D, K2069fs*16, L2092fs*57, P2033fs*62, P2115fs*10, P42fs*194, R1589Q, R2031fs*54, R6fs*28, S1448fs*115, S157fs*5, splice site 119−156_197+47del282, splice site 1606+1G>A, splice site 5668−1G>T, T1098fs*174, or W1425*; (v) the alteration in a EP300 gene comprises one or more of: H2324fs*55, M1470fs*26, R1187H, splice site 1282+1G>A, C1385F, D1399N, H2324fs*29, L2303fs*74, L415P, R86*, C1408*, C1738*, E643fs*2, G54*, K1469fs*3, M1339fs*26, N1236fs*41, N419fs*12, Q2282*, Q498*, Q501fs*6, R1055*, R1281*, R1312*, R1627W, R2185*, R2263*, R2330fs*49, R580Q, R648*, S1214Y, S19fs*19, S2271fs*8, splice site 1169−2A>G, splice site 2379+1G>C, splice site 3671+1G>A, splice site 4453−2A>G, T1021fs*3, or Y1467H; (w) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*; (x) the alteration in a MEN1 gene comprises one or more of: R521fs*43, E184V, L105fs*13, R457Q, R521fs*15, R532*, or splice site 799−9G>A; and/or (y) the alteration in a BCOR gene comprises one or more of: P1587fs*53, Q1174fs*8, K1173fs*31, G400fs*42, R810*, Q1272fs*20, R1480*, S158fs*28, S336fs*45, C1329fs*45, D328fs*50, E1025*, E1030fs*48, E1182fs*6, E485fs*42, G154*, G906fs*5, G95fs*16, H1179fs*1, I1290fs*2, K1061fs*52, K1137fs*4, K1271fs*64, K1330*, K839fs*17, L279fs*21, N1425S, N390fs*53, N529fs*28, P602fs*67, P931fs*15, Q1174*, Q1274*, Q348*, Q430*, Q600*, R1053fs*26, R1181fs*1, R1498fs*36, R976*, S1371L, S336fs*42, splice site 166−2A>C, splice site 2997+1G>T, splice site 3239−2A>G, splice site 4072−2A>G, T1331fs*4, or V806fs*10. In some embodiments, the CRC is microsatellite stable (MSS) and/or does not comprise a POLE and/or POLD1 alteration, and wherein the CRC comprises an alteration in a CTNNB1 and/or MAP3K1 gene, optionally wherein: (a) the alteration in a CTNNB1 gene comprises one or more of: T41A, S45F, Q773*, R587*, S33C, D32N, N387K, S45P, T257L, W25*, D17_Q78del, D17_T75del, I35_G38del, I35S, K19_S37>N, K335L, M8_V79del, R376H, R582Q, R582W, R90*, R95*, S33F, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242−39del684, splice site 14−338_81>TTAC, splice site 14−39_225del251, splice site 14−48_241+12del288, splice site 14−6_241+74del308, splice site 14−69_242−10del488, splice site 14−7_89del83, splice site 14−80_241+22>CAT, splice site 14−91_241+25del344, splice site 1954+1G>A, splice site 1954+1G>T, splice site 1955−1G>A, splice site 241+1G>C, splice site 32_241+94del304, splice site 60_287del428, splice site 65_274del410, splice site 74_241+52del220, splice site 78_241+11del175, splice site 87_242−79del277, splice site 98_241+8>A, T41I, V22_S33del, V22_S37del, W25_I35>C, W383C, W383R, or W776*; and/or (b) the alteration in a MAP3K1 gene comprises one or more of: E126*, E1293fs*3, E788*, H114fs*50, L380fs*4, P74fs*3, Q1022*, R532*, S101fs*63, splice site 483−1G>A, T1145fs*6, or T457fs*31.

In some embodiments of any of the aspects or embodiments provided herein, the CRC is a primary CRC or is metastatic.

In some embodiments of any of the aspects or embodiments provided herein, the CRC comprises one or more KRAS alterations, optionally wherein the one or more KRAS alterations comprise a G12C, G12D, G12V, or G13D amino acid substitution.

In some embodiments of any of the aspects or embodiments provided herein, the CRC is mismatch repair deficient (dMMR).

In some embodiments of any of the aspects or embodiments provided herein, the CRC is not dMMR CRC.

In some embodiments of any of the aspects or embodiments provided herein, the CRC is an adenocarcinoma, a carcinoid tumor, a familial CRC, a gastrointestinal stromal tumor (GIST), a colorectal lymphoma, a squamous cell carcinoma, a leiomyosarcoma, or an angiosarcoma.

In some embodiments of any of the aspects or embodiments provided herein, the one or more biomarkers comprise an MSI-H status.

In some embodiments of any of the aspects or embodiments provided herein, the MSI-H status is detected by sequencing, a polymerase chain reaction (PCR) amplification technique, a non-PCR amplification technique, an isothermal amplification technique, a capillary electrophoresis method, immunohistochemistry, or any combination thereof. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS).

In some embodiments of any of the aspects or embodiments provided herein, the MSI-H status is detected based on DNA sequencing of up to about 114 loci; and/or wherein the MSI-H status is determined if at least about 1.24% of analyzed loci have instability.

In some embodiments of any of the aspects or embodiments provided herein, the one or more biomarkers comprise a high TMB.

In some embodiments of any of the aspects or embodiments provided herein, the high TMB comprises a TMB of ≥10 mutations/Megabase (mut/Mb).

In some embodiments of any of the aspects or embodiments provided herein, the high TMB is detected based on about 0.79 megabases (Mb) of sequenced DNA.

In some embodiments of any of the aspects or embodiments provided herein, the high TMB is detected based on about 0.80 Mb of sequenced DNA.

In some embodiments of any of the aspects or embodiments provided herein, the high TMB is detected on between about 0.83 Mb and about 1.14 Mb of sequenced DNA, or between about 0.8 Mb and about 1.1 Mb of sequenced DNA.

In some embodiments of any of the aspects or embodiments provided herein, the high TMB is detected based on about 1.1 Mb of sequenced DNA.

In some embodiments of any of the aspects or embodiments provided herein, the high TMB is detected based on up to about 1.24 Mb of sequenced DNA.

In some embodiments of any of the aspects or embodiments provided herein, the high TMB is detected based on up to about 1.1 Mb of sequenced DNA.

In some embodiments of any of the aspects or embodiments provided herein, high TMB comprises a TMB of at least about 10 mut/Mb, at least about 15 mut/Mb, at least about 20 mut/Mb, at least about 25 mut/Mb, at least about 30 mut/Mb, at least about 35 mut/Mb, at least about 40 mut/Mb, at least about 45 mut/Mb, at least about 50 mut/Mb, at least about 55 mut/Mb, at least about 60 mut/Mb, at least about 65 mut/Mb, at least about 70 mut/Mb, at least about 75 mut/Mb, at least about 80 mut/Mb, at least about 85 mut/Mb, at least about 90 mut/Mb, at least about 95 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

In some embodiments of any of the aspects or embodiments provided herein, the high TMB is detected by sequencing, whole exome sequencing, whole genome sequencing, gene-targeted sequencing, or next-generation sequencing.

In some embodiments of any of the aspects or embodiments provided herein, the one or more biomarkers comprise a PD-L1 positive status.

In some embodiments of any of the aspects or embodiments provided herein, the PD-L1 positive status is detected based on PD-L1 protein expression. In some embodiments, PD-L1 protein expression is determined using an immunohistochemistry assay. In some embodiments, the immunohistochemistry assay is a DAKO PD-L1 22C3 assay. In some embodiments, PD-L1 expression is assessed based on a tumor proportion score (TPS). In some embodiments, the PD-L1 positive status comprises a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%. In some embodiments, the PD-L1 positive status comprises a TPS of between about 1% and about 49%. In some embodiments, the PD-L1 positive status comprises a TPS of at least about 1%. In some embodiments, the PD-L1 positive status comprises a TPS of at least about 25%, at least about 50%, or at least about 75%. In some embodiments, PD-L1 expression is assessed based on a combined positive score (CPS). In some embodiments, the PD-L1 positive status comprises a CPS of at least about 1 or at least about 10. In some embodiments, the immunohistochemistry assay is a VENTANA SP 142 assay. In some embodiments, PD-L1 expression is assessed based on the proportion of tumor area occupied by PD-L1-expressing tumor-infiltrating immune cells of any intensity (IC), or the percentage of PD-L1-expressing tumor cells of any intensity (TC). In some embodiments, the CRC is PD-L1 positive. In some embodiments, the PD-L1 positive status comprises a TC or IC of at least about 1%.

In some embodiments of any of the aspects or embodiments provided herein, the one or more biomarkers comprise an alteration in the one or more genes.

In some embodiments of any of the aspects or embodiments provided herein, the alteration in the one or more genes comprises a point mutation; an insertion-deletion (indel); an in-frame deletion of one or more codons; an intragenic deletion; an intragenic insertion; a deletion of a full gene; an inversion; an interchromosomal or intrachromosomal translocation; a tandem duplication; a gene fusion; a genomic rearrangement; a splice site mutation; and/or a gene amplification or duplication.

In some embodiments of any of the aspects or embodiments provided herein: (a) the alteration in BRAF is a V600E, D594G, G469A, N581S, G466V, K483E, L485F, L485S, or T241M alteration, or any combination thereof, (b) the alteration in PTEN is a K267fs*9, N323fs*21, R233*, R130*, R130Q, E299*, R173H, T319fs*1, C136Y, C250fs*2, D24fs*20, E157fs*23, E242fs*9, F90fs*9, H93R, I33del, K164fs*3, K330*, L247*, L325P, L57fs*6, N323fs*2, N340fs*3, N63fs*11, P95L, Q171*, Q219*, Q245*, Q261*, R234W, S59*, splice site 209+5G>A, splice site 210−1G>C, splice site 626_634+2delGAACTTGCAGT, splice site 79+1G>A, T131N, or V133I alteration, or any combination thereof; (c) the alteration in RNF43 is a G659fs*41, R117fs*41, R225fs*194, R132*, R145*, R330*, Y332*, A193fs*6, A273fs*147, A78T, E258fs*162, G257fs*162, K181fs*4, L311fs*132, M1I, P660fs*87, Q153*, Q233*, Q426*, Q426fs*77, Q8*, R225fs*195, R337*, splice site 375+1G>A, V271fs*11, V299fs*120, V479fs*25, W159*, W302*, Y248*, or Y332fs*110V alteration, or any combination thereof (d) the alteration in ASXL1 is a G645fs*58, G646fs*12, R693*, A627fs*8, E41K, E676*, G646fs*58, L983fs*8, P1377fs*3, P763fs*12, Q561fs*1, or S892fs*16 alteration, or any combination thereof; (e) the alteration in CREBBP is a I1084fs*15, P1423fs*36, A1824T, Q1209fs*25, G1145fs*23, R714H, I1084fs*3, K668fs*27, L555fs*7, P2094L, P937fs*61, Q278*, Q911*, R1446H, S801*, splice site 3836+1G>A, or Y1503H alteration, or any combination thereof; (f) the alteration in MLL2 is a P2354fs*30 9, G1235fs*95, P647fs*283, T382fs*20, A2205fs*59, C2436fs*49, C346fs*17, D2769N, E2962fs*42, F1790fs*12, G2265fs*21, H77fs*53, I977fs*23, K1686fs*36, K304fs*30, L1020fs*36, L5183fs*16, P1460fs*46, P2206fs*58, P367fs*35, P4380fs*4, P444fs*2, P4968fs*27, P506fs*424, P583fs*347, P648fs*2, Q1377R, Q3811fs*201, R1252*, R1687fs*4, R2771*, R2830*, R4238C, R4904*, R5048H, R5282*, R755fs*3, S1107fs*12, S1684T, S2910fs*32, S4507fs*12, S4789fs*27, splice site 14644−1G>T, splice site 16413−2A>G, T209fs*11, V1244fs*86, V1670fs*52, or V4799M alteration, or any combination thereof; (g) the alteration in BCORL1 is a P1681fs*20, A1166fs*56, A971fs*4, E1655*, E619*, G1682fs*4, K1207N, P323fs*95, Q1001fs*49, R1297*, R1299*, R1420*, or W1105* alteration, or any combination thereof; (h) the alteration in ATR is a I774fs*5, F1091fs*28, F1134fs*6, I774fs*3, E2579*, F2168*, I691fs*5, K446fs*11, K773fs*3, R2001*, R223fs*1, R2547*, or W1591* alteration, or any combination thereof; (i) the alteration in SPEN is a A2105fs*33, R806fs*14, A2105fs*18, H2985fs*199, I1052fs*40, I577fs*37, N2002fs*20, P2495fs*4, P2839fs*50, P3631fs*3, Q253fs*109, R1936*, R2332H, or V1294fs*7 alteration, or any combination thereof, (j) the alteration in BRCA1 is a K654fs*47, Q1756fs*74, Q74*, R1203*, S324fs*16, splice site 4185+2_4185+22>A, or splice site 442−2A>G alteration, or any combination thereof; (k) the alteration in BRCA2 is a E2981K, E2981fs*7, N1784fs*7, R2842C, T3033fs*29, A1237fs*2, C3233fs*15, D252fs*24, E2144*, E3316fs*2, E597*, E866*, M2393fs*19, N1287fs*6, N1784fs*3, N2189fs*2, R2034H, R2318*, T3085fs*26, V1862fs*1, or W2830* alteration, or any combination thereof; and/or (l) the alteration in MSH6 is a F1088fs*5, F1088fs*2, F1088fs*3, E946*, A1236fs*4, A1320fs*5, C694fs*4, K1140fs*24, K247fs*32, L1356fs*1, R240*, R248fs*8, R298*, or R361H alteration, or any combination thereof.

In some embodiments of any of the aspects or embodiments provided herein, the CRC is microsatellite stable (MSS). In some embodiments of any of the aspects or embodiments provided herein, the CRC does not have an alteration in a POLE and/or POLD1 gene. In some embodiments, the one or more genes comprise one or more of BRAF, PTEN, or RNF43; optionally wherein: (a) the alteration in BRAF is a V600E, D594G, N581S, G466V, G469A, K483E, L485F, or L485S alteration, or any combination thereof; (b) the alteration in PTEN is a R233*, C136Y, D24fs*20, E242fs*9, E299*, H93R, K330*, L247*, L57fs*6, N323fs*2, N340fs*3, N63fs*11, P95L, Q171*, Q219*, Q245*, R130*, R130Q, splice site 209+5G>A, splice site 210−1G>C, splice site 626_634+2delGAACTTGCAGT, T131N, or V133I alteration, or any combination thereof; and/or (c) the alteration in RNF43 is a Y332*, A193fs*6, A273fs*147, E258fs*162, M1I, P660fs*87, Q233*, Q426fs*77, Q8*, R117fs*41, R145*, R337*, splice site 375+1G>A, V271fs*11, W159*, W302*, Y248*, or Y332fs*110 alteration, or any combination thereof.

In some embodiments of any of the aspects or embodiments provided herein, the alteration in the one or more genes is detected by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments of any of the aspects or embodiments provided herein, the methods further comprise selectively enriching for one or more nucleic acid molecules in a sample from the individual comprising nucleotide sequences corresponding to the one or more genes; wherein the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the one or more genes and producing nucleic acid hybrids; and (b) isolating the nucleic acid hybrids to produce the enriched sample. In some embodiments, the selectively enriching comprises amplifying one or more nucleic acid molecules in a sample from the individual comprising nucleotide sequences corresponding to the one or more genes in the sample; thereby producing the enriched sample. In some embodiments, the methods further comprise sequencing the enriched sample to detect the alteration in the one or more genes.

In some embodiments of any of the aspects or embodiments provided herein, the methods further comprise (a) optionally, ligating one or more adapters onto nucleic acid molecules in a sample from the individual, thereby generating ligated nucleic acids; (b) optionally, amplifying nucleic acids from the ligated nucleic acids; (c) optionally, capturing or enriching from the amplified nucleic acids a plurality of nucleic acids comprising nucleotide sequences corresponding to the one or more genes; (d) sequencing, by a sequencer, the plurality of nucleic acids to obtain a plurality of sequence reads corresponding to the one or more genes; (e) analyzing the plurality of sequence reads; and (f) based on the analysis, detecting the alteration in the one or more genes. In some embodiments, the plurality of nucleic acids comprising nucleotide sequences corresponding to the one or more genes is captured from the amplified nucleic acids by hybridization with one or more bait molecules. In some embodiments, the capturing comprises: (a) combining the one or more bait molecules with the amplified nucleic acids, thereby hybridizing the one or more bait molecules to nucleic acids comprising nucleotide sequences corresponding to the one or more genes and producing nucleic acid hybrids; and (b) isolating the nucleic acid hybrids. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the one or more genes and/or the alteration in the one or more genes. In some embodiments, the one or more adapters comprise amplification primers, flow cell adapter sequences, substrate adapter sequences, sample index sequences, or unique molecular identifier (UMI) sequences. In some embodiments, the sequencer comprises a next generation sequencer. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS).

In some embodiments of any of the aspects or embodiments provided herein, the methods further comprise generating a molecular profile for the individual, based, at least in part, on detecting or acquiring knowledge of the one or more biomarkers in the one or more samples from the individual. In some embodiments, the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual further comprises results from a nucleic acid sequencing-based test. In some embodiments, the method further comprises selecting a treatment, administering a treatment, or applying a treatment to the individual based on the generated molecular profile.

In some embodiments of any of the aspects or embodiments provided herein, the methods comprise generating a report indicating the presence or absence of the one or more biomarkers in one or more samples from the individual.

In some embodiments of any of the aspects or embodiments provided herein, the methods comprise generating, by the one or more processors, a report indicating the presence or absence of the alteration in the one or more genes in a sample from the individual.

In some embodiments of any of the aspects or embodiments provided herein, the methods comprise transmitting the report to the individual, a caregiver, a healthcare provider, a physician, an oncologist, an electronic medical record system, a hospital, a clinic, a third-party payer, an insurance company, or a government office. In some embodiments, the report is transmitted via a computer network or a peer-to-peer connection. In some embodiments, the one or more bait molecules comprise a capture nucleic acid molecule configured to hybridize to a nucleotide sequence corresponding to the one or more genes. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the one or more bait molecules are conjugated to an affinity reagent and/or to a detection reagent. In some embodiments, the affinity reagent is an antibody, an antibody fragment, or biotin, and/or wherein the detection reagent is a fluorescent marker. In some embodiments, the capture nucleic acid molecule comprises a DNA, RNA, or mixed DNA/RNA molecule.

In some embodiments of any of the aspects or embodiments provided herein, detecting, acquiring knowledge of, or identifying the presence of the one or more biomarkers in the one or more samples from the individual identifies or predicts the individual as being at risk for early CRC recurrence. In some embodiments, detecting, acquiring knowledge of, or identifying the presence of the one or more biomarkers in the one or more samples from the individual identifies or predicts the individual as being at greater risk for early CRC recurrence, as compared to an individual with a CRC that does not comprise the one or more biomarkers. In some embodiments, detecting, acquiring knowledge of, or identifying the presence of the one or more biomarkers in the one or more samples from the individual identifies or predicts the individual as being at risk for CRC recurrence within about one year or less from the time of initial diagnosis of the CRC. In some embodiments, detecting, acquiring knowledge of, or identifying the presence of the one or more biomarkers in the one or more samples from the individual identifies or predicts the individual as being at greater risk for CRC recurrence within about one year or less from the time of initial diagnosis of the CRC, as compared to an individual with a CRC that does not comprise the one or more biomarkers.

In some embodiments of any of the aspects or embodiments provided herein, further comprises administering an anti-cancer therapy to the individual based, at least in part, on detecting, acquiring knowledge, or identifying the presence of the one or more biomarkers in the one or more samples from the individual.

In some embodiments of any of the aspects or embodiments provided herein, the chemotherapy comprises 5-fluorouracil (5-FU), irinotecan, oxaliplatin, capecitabine, or Trifluridine/Tipiracil, or any combination thereof, and/or wherein the method further comprises administering the chemotherapy to the individual.

In some embodiments of any of the aspects or embodiments provided herein, detecting, acquiring knowledge of, or identifying the presence of the one or more biomarkers in the one or more samples from the individual identifies the individual as one who should be monitored or assessed for CRC recurrence more frequently, as compared to an individual with a CRC that does not comprise the one or more biomarkers.

In some embodiments of any of the aspects or embodiments provided herein, detecting, acquiring knowledge of, or identifying the presence of the one or more biomarkers in the one or more samples from the individual identifies the individual as a candidate to receive a more aggressive anti-cancer therapy for CRC and/or an anti-cancer therapy for CRC of greater duration, as compared to an individual with a CRC that does not comprise the one or more biomarkers.

In some embodiments of any of the aspects or embodiments provided herein, detecting, acquiring knowledge of, or identifying the presence of the one or more biomarkers in the one or more samples from the individual identifies the individual as one who: (a) should be administered a standard-of-care treatment for CRC, optionally a more aggressive standard-of-care treatment for CRC and/or a standard-of-care treatment for CRC of greater duration, as compared to an individual with a CRC that does not comprise the one or more biomarkers; or (b) should be administered an anti-cancer therapy other than a standard-of-care treatment for CRC, or an anti-cancer therapy combined with a standard-of-care treatment for CRC. In some embodiments, the standard-of-care treatment for CRC comprises a chemotherapy combined with an anti-VEGF agent or an anti-EGFR agent.

In some embodiments of any of the aspects or embodiments provided herein, the chemotherapy comprises a leucovorin calcium (folinic acid), fluorouracil, and oxaliplatin combination (FOLFOX). In some embodiments, the anti-VEGF agent is an anti-VEGF antibody, optionally wherein the anti-VEGF antibody is bevacizumab. In some embodiments, the anti-EGFR agent is an anti-EGFR antibody, optionally wherein the anti-EGFR antibody is cetuximab.

In some embodiments of any of the aspects or embodiments provided herein, the anti-cancer therapy comprises a chemotherapy, a radiation therapy, an immunotherapy, a targeted therapy, a surgery, or any combination thereof, optionally wherein the chemotherapy comprises 5-fluorouracil (5-FU), irinotecan, oxaliplatin, capecitabine, or Trifluridine/Tipiracil, or any combination thereof.

In some embodiments of any of the aspects or embodiments provided herein, the anti-cancer therapy comprises a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), a tyrosine kinase inhibitor therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the aspects or embodiments provided herein, the one or more biomarkers comprise an MSI-H status, a high TMB, and/or a PD-L1 positive status, and wherein the anti-cancer therapy comprises an immunotherapy.

In some embodiments of any of the aspects or embodiments provided herein: the one or more biomarkers comprise an alteration in a BRAF gene, and wherein the anti-cancer therapy comprises a BRAF-targeted therapy, an EGFR-targeted therapy, or a combination thereof, the one or more biomarkers comprise an alteration in a PTEN gene, and wherein the anti-cancer therapy comprises a PTEN-targeted therapy; the one or more biomarkers comprise an alteration in a RNF43 gene, and wherein the anti-cancer therapy comprises a RNF43-targeted therapy, a BRAF-targeted therapy, an EGFR-targeted therapy, a MEK-targeted therapy, or a combination thereof, the one or more biomarkers comprise an alteration in an ASXL1 gene, and wherein the anti-cancer therapy comprises an ASXL1-targeted therapy; the one or more biomarkers comprise an alteration in a CREBBP gene, and wherein the anti-cancer therapy comprises a CREBBP-targeted therapy; the one or more biomarkers comprise an alteration in an MLL2 gene, and wherein the anti-cancer therapy comprises an MLL2-targeted therapy; the one or more biomarkers comprise an alteration in a BCORL1 gene, and wherein the anti-cancer therapy comprises a BCORL1-targeted therapy; the one or more biomarkers comprise an alteration in an ATR gene, and wherein the anti-cancer therapy comprises an ATR-targeted therapy; the one or more biomarkers comprise an alteration in a SPEN gene, and wherein the anti-cancer therapy comprises a SPEN-targeted therapy; the one or more biomarkers comprise an alteration in a BRCA1 gene, and wherein the anti-cancer therapy comprises a BRCA1-targeted therapy; the one or more biomarkers comprise an alteration in a BRCA2 gene, and wherein the anti-cancer therapy comprises a BRCA2-targeted therapy; and/or the one or more biomarkers comprise an alteration in a MSH6 gene, and wherein the anti-cancer therapy comprises a MSH6-targeted therapy. In some embodiments, the BRAF-targeted therapy comprises an anti-EGFR agent or a combination of a chemotherapy and an anti-EGFR agent, optionally wherein the anti-EGFR agent is an anti-EGFR antibody.

In some embodiments of any of the aspects or embodiments provided herein, the anti-cancer therapy is a first-line anti-cancer therapy for the CRC.

In some embodiments of any of the aspects or embodiments provided herein, the methods further comprise obtaining the one or more samples from the individual. In some embodiments, one or more of the samples from the individual are obtained about 3 months or less from the time of diagnosis of a CRC in the individual. In some embodiments, one or more of the samples from the individual are obtained prior to the individual having received a treatment for the CRC.

In some embodiments of any of the aspects or embodiments provided herein, one or more of the samples from the individual comprise or are derived from a tissue biopsy sample, a liquid biopsy sample, or a normal control. In some embodiments, one or more of the samples from the individual are or are derived from a liquid biopsy sample and comprise blood, plasma, cerebrospinal fluid, sputum, stool, urine, or saliva. In some embodiments, one or more of the samples from the individual are a liquid biopsy and comprise circulating tumor cells (CTCs). In some embodiments, one or more of the samples from the individual are a liquid biopsy sample and comprise cell-free DNA (cfDNA), circulating tumor DNA (ctDNA), or any combination thereof.

In some embodiments of any of the aspects or embodiments provided herein, one or more of the samples from the individual comprise a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some aspects, provided herein is an anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and wherein detection of the one or more biomarkers in the one or more samples identifies the individual as being at risk for CRC recurrence.

In some aspects, provided herein is an anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and wherein detection of the one or more biomarkers in the one or more samples identifies the individual as being at risk for CRC recurrence.

In some aspects, provided herein is an anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from: (i) an MSI-H status, (ii) a high TMB, and (iii) a PD-L1 positive status; and wherein detection of the one or more biomarkers in the one or more samples identifies the individual as being at risk for CRC recurrence.

In some aspects, provided herein is a chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and wherein detection of the one or more biomarkers in the one or more samples identifies the individual as being at risk for CRC recurrence.

In some aspects, provided herein is a chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein detection of the one or more biomarkers in the one or more samples identifies the individual as being at risk for CRC recurrence.

In some aspects, provided herein is a chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from: (i) an MSI-H status, (ii) a high TMB, and (iii) a PD-L1 positive status; and wherein detection of the one or more biomarkers in the one or more samples identifies the individual as being at risk for CRC recurrence.

In some aspects, provided herein is an anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof, and wherein detection of the one or more biomarkers in the one or more samples identifies the individual as being at risk for CRC recurrence.

In some aspects, provided herein is an anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein detection of the one or more biomarkers in the one or more samples identifies the individual as being at risk for CRC recurrence.

In some aspects, provided herein is an anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, and (iii) a PD-L1 positive status; and wherein detection of the one or more biomarkers in the one or more samples identifies the individual as being at risk for CRC recurrence.

In some aspects, provided herein is a chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, or SPEN, or any combination thereof; and wherein detection of the one or more biomarkers in the one or more samples identifies the individual as being at risk for CRC recurrence.

In some aspects, provided herein is a chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, (iii) a PD-L1 positive status, and (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and wherein detection of the one or more biomarkers in the one or more samples identifies the individual as being at risk for CRC recurrence.

In some aspects, provided herein is a chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from: (i) an MSI-H status, (ii) a high TMB, and (iii) a PD-L1 positive status; and wherein detection of the one or more biomarkers in the one or more samples identifies the individual as being at risk for CRC recurrence.

In some aspects, provided herein is a system, comprising: a memory configured to store one or more program instructions, and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a CRC; (b) analyze the plurality of sequence reads for the presence of one or more biomarkers, wherein the one or more biomarkers are selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, or SPEN, or any combination thereof, and (c) detect, based on the analyzing, the one or more biomarkers in the one or more samples, wherein detection of the one or more biomarkers in the one or more samples identifies the individual as being at risk for CRC recurrence.

In some aspects, provided herein is a system, comprising: a memory configured to store one or more program instructions, and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a CRC; (b) analyze the plurality of sequence reads for the presence of one or more biomarkers, wherein the one or more biomarkers are selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and (c) detect, based on the analyzing, the one or more biomarkers in the one or more samples, wherein detection of the one or more biomarkers in the one or more samples identifies the individual as being at risk for CRC recurrence.

In some aspects, provided herein is a system, comprising: a memory configured to store one or more program instructions, and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a CRC; (b) analyze the plurality of sequence reads for the presence of one or more biomarkers, wherein the one or more biomarkers are selected from: (i) an MSI-H status, and (ii) a high TMB; and (c) detect, based on the analyzing, the one or more biomarkers in the one or more samples, wherein detection of the one or more biomarkers in the one or more samples identifies the individual as being at risk for CRC recurrence.

In some aspects, provided herein is a non-transitory computer readable storage medium comprising one or more programs executable by one or more computer processors for performing a method, the method comprising: (a) obtaining, using the one or more processors, a plurality of sequence reads of one or more nucleic acid molecules, wherein the one or more nucleic acid molecules are derived from one or more samples obtained from an individual having a CRC; (b) analyzing, using the one or more processors, the plurality of sequence reads for the presence of one or more biomarkers, wherein the one or more biomarkers are selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, or SPEN, or any combination thereof, and (c) detecting, using the one or more processors and based on the analyzing, the one or more biomarkers in the one or more samples, wherein detection of the one or more biomarkers in the one or more samples identifies the individual as being at risk for CRC recurrence.

In some aspects, provided herein is a non-transitory computer readable storage medium comprising one or more programs executable by one or more computer processors for performing a method, the method comprising: (a) obtaining, using the one or more processors, a plurality of sequence reads of one or more nucleic acid molecules, wherein the one or more nucleic acid molecules are derived from one or more samples obtained from an individual having a CRC; (b) analyzing, using the one or more processors, the plurality of sequence reads for the presence of one or more biomarkers, wherein the one or more biomarkers are selected from: (i) an MSI-H status, (ii) a high TMB, and (iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and (c) detecting, using the one or more processors and based on the analyzing, the one or more biomarkers in the one or more samples, wherein detection of the one or more biomarkers in the one or more samples identifies the individual as being at risk for CRC recurrence.

In some aspects, provided herein is a non-transitory computer readable storage medium comprising one or more programs executable by one or more computer processors for performing a method, the method comprising: (a) obtaining, using the one or more processors, a plurality of sequence reads of one or more nucleic acid molecules, wherein the one or more nucleic acid molecules are derived from one or more samples obtained from an individual having a CRC; (b) analyzing, using the one or more processors, the plurality of sequence reads for the presence of one or more biomarkers, wherein the one or more biomarkers are selected from: (i) an MSI-H status, and (ii) a high TMB; and (c) detecting, using the one or more processors and based on the analyzing, the one or more biomarkers in the one or more samples, wherein detection of the one or more biomarkers in the one or more samples identifies the individual as being at risk for CRC recurrence.

In some embodiments of any of the aspects provided herein, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and further optionally wherein the massively parallel sequencing technique comprises next generation sequencing (NGS).

In some embodiments of any of the aspects or embodiments provided herein, the one or more program instructions when executed by the one or more processors are further configured to generate, based at least in part on the detecting, a molecular profile for the sample.

In some embodiments of any of the aspects or embodiments provided herein, the method further comprises generating, based at least in part on the detecting, a molecular profile for the sample. In some embodiments, the individual is administered a treatment based at least in part on the molecular profile; optionally wherein the treatment comprises an anti-cancer therapy. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, a PD-L1 expression test, or any combination thereof. In some embodiments, the molecular profile further comprises results from a nucleic acid sequencing-based test. In some embodiments, the molecular profile further indicates the presence or absence of a PD-L1 positive status in a sample from the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts a process flowchart for selection of the colorectal cancer (CRC) patient cohorts used in Example 1 herein. CGDB, clinico-genomic database; dx, diagnosis; mo, month; yr, year.

FIGS. 2A-2D provide a comparison of genomic alterations in CRC patients with Stage I-III and Stage IV disease. FIG. 2A is a bar plot showing the prevalence of actionable alterations in patients with Stage I-III and Stage IV CRC. For each alteration, data for Stage IV are shown on the left, and data for Stage I-III are shown on the right. Statistically significant (adjusted p-value<0.05) differences are noted by asterisks. FIG. 2B shows the prevalence of the indicated KRAS mutations among patients with Stage I-III and Stage IV CRC. FIG. 2C shows a volcano plot depicting gene alterations identified in patients with Stage I-III and Stage IV CRC. The dashed horizontal line indicates the statistical significance threshold (adjusted p-value≤0.05). OR, odds ratio; FDR, false discovery rate. FIG. 2D shows a bar plot of the prevalence of gene alterations statistically significant in FIG. 2C in microsatellite stable (MSS) patients with no POLE/POLD1 mutations. For each alteration, data for Stage IV are shown on the left, and data for Stage I-III are shown on the right. Statistically significant (adjusted p-value<0.05) differences are noted by asterisks.

FIGS. 3A-3C provide a comparison of genomic alterations in MSS and non-POLE/POLD1 mutated CRC patients with Stage I-III and Stage IV disease. FIG. 3A is a bar plot showing the prevalence of actionable alterations in patients with Stage I-Ill and Stage IV disease who are MSS and non-POLE/POLD1 mutated. For each alteration, data for Stage IV are shown on the left, and data for Stage I-Ill are shown on the right. FIG. 3B shows the prevalence of the indicated KRAS mutations among patients with Stage I-III and Stage IV disease who are MSS and non-POLE/POLD1 mutated. FIG. 3C shows a volcano plot depicting gene alterations identified in patients with Stage I-III and Stage IV disease who are MSS and non-POLE/POLD1 mutated. The dashed horizontal line indicates the statistical significance threshold (adjusted p-value≤0.05). OR, odds ratio; FDR, false discovery rate.

FIGS. 4A-4D provide a comparison of genomic alterations in patients with Stage I-III CRC whose disease recurred early (≤1 year after initial diagnosis) or late (>1 year after initial diagnosis). FIG. 4A is a bar plot showing the prevalence of actionable alterations in patients with Stage I-III CRC whose disease recurred early or late. For each alteration, data for Stage IV are shown on the left, and data for Stage I-III are shown on the right. Statistically significant (adjusted p-value<0.05) differences are noted by asterisks. FIG. 4B shows the prevalence of the indicated KRAS mutations among patients with early or late CRC recurrence. FIG. 4C shows a volcano plot depicting gene alterations identified in patients with Stage I-Ill CRC with early or late recurrence. The dashed horizontal line indicates the statistical significance threshold (adjusted p-value≤0.05). OR, odds ratio; FDR, false discovery rate. FIG. 4D shows a bar plot of the prevalence of gene alterations statistically significant in FIG. 4C in MSS patients with no POLE/POLD1 mutations that had early or late CRC recurrence. For each alteration, data for late recurrence are shown on the left, and data for early recurrence are shown on the right. Statistically significant (adjusted p-value<0.05) differences are noted by asterisks.

FIGS. 5A-5C provide a comparison of genomic alterations in MSS and non-POLE/POLD1 mutated, Stage I-Ill CRC patients whose disease recurred early (≤1 year after initial diagnosis) or late (>1 year after initial diagnosis). FIG. 5A is a bar plot showing the prevalence of actionable alterations in MSS and non-POLE/POLD1 mutated CRC patients with early or late disease recurrence. For each alteration, data for late recurrence are shown on the left, and data for early recurrence are shown on the right. FIG. 5B shows the prevalence of the indicated KRAS mutations in MSS and non-POLE/POLDI mutated CRC patients with early or late disease recurrence. FIG. 5C shows a volcano plot depicting gene alterations identified in MSS and non-POLE/POLD1 mutated CRC patients with early or late disease recurrence. The dashed horizontal line indicates the statistical significance threshold (adjusted p-value≤0.05). OR, odds ratio; FDR, false discovery rate.

FIG. 6 depicts an exemplary device, in accordance with some embodiments.

FIG. 7 depicts an exemplary system, in accordance with some embodiments.

FIG. 8 depicts a block diagram of an exemplary process for detecting one or more biomarkers of CRC recurrence, in accordance with some embodiments.

### DETAILED DESCRIPTION

The present disclosure relates generally to detecting biomarkers that may be used for assessing colorectal cancer (CRC) recurrence risk, as well as methods of treatment, and uses related thereto.

The disclosure describes results of comprehensive genomic profiling (CGP) of CRC patients to identify biomarkers associated with CRC recurrence. These analyses identified that (a) a microsatellite instability high (MSI-H) status, (b) a high tumor mutational burden (TMB), (c) a PD-L1 positive status, and (d) an alteration in BRAF, PTEN, RNF43, ASXL1, CREBBP, MLL2, BCORL1, ATR, SPEN, BRCA1, BRCA2, or MSH6 were associated with recurrence of CRC (see Example 1, herein). Without wishing to be bound by theory, it is thought that the one or more biomarkers associated with recurrence of CRC, such as one or more, or all, of (a) a microsatellite instability high (MSI-H) status, (b) a high tumor mutational burden (TMB), (c) a PD-L1 positive status, and (d) an alteration in BRAF, PTEN, RNF43, ASXL1, CREBBP, MLL2, BCORL1, ATR, SPEN, BRCA1, BRCA2, or MSH6, or any combination thereof, may identify patients who are at risk for CRC recurrence and could benefit from treatment with anti-cancer therapies, e.g., as described in greater detail below.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning. A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

As used in this specification and the appended claims, the singular forms “a”, “an” and “the” include plural referents unless the context clearly dictates otherwise. Thus, for example, reference to “a molecule” optionally includes a combination of two or more such molecules, and the like.

The terms “about” and “approximately” as used herein refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule”, as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(O)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the disclosure may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-31 0 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 11 9-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The terms “homology” or “identity,” as used herein, refer to sequence similarity between two polynucleotide sequences or between two polypeptide sequences. The phrases “percent identity or homology” and “% identity or homology” refer to the percentage of sequence similarity found in a comparison of two or more polynucleotide sequences or two or more polypeptide sequences. Identity or similarity can be determined by comparing a position in each sequence that can be aligned for purposes of comparison. When a position in the compared sequences is occupied by the same nucleotide base or amino acid, then the molecules are identical at that position.

The term “detection” includes any means of detecting, including direct and indirect detection. The term “biomarker” as used herein (e.g., (a) a microsatellite instability high (MSI-H) status, (b) a high tumor mutational burden (TMB), (c) a PD-L1 positive status, and/or (d) an alteration in one or more genes) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein, generally refers to the process of producing multiple copies of a desired sequence. “Multiple copies” means at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” “normal control”, or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of assessment of biomarkers, one may use the results of an analysis or protocol assessing a biomarker to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

As used herein, by “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen wherein the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker. In certain embodiments, the manufacture or kit is promoted, distributed, or sold as a unit for performing the methods described herein.

The phrases “based on”, “responsive to”, and the like, when used herein mean that the information about one or more biomarkers is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. METHODS, SYSTEMS, AND DEVICES

The present disclosure relates generally to assessing one or more biomarkers associated with colorectal cancer (CRC) recurrence, as well as methods, systems and devices related thereto. Such biomarkers include one or more, or all, of: (a) a microsatellite instability high (MSI-H) status, (b) a high tumor mutational burden (TMB), (c) a PD-L1 positive status, and (d) an alteration in BRAF, PTEN, RNF43, ASXL1, CREBBP, MLL2, BCORL1, ATR, SPEN, BRCA1, BRCA2, or MSH6, or any combination thereof.

As described in greater detail herein, acquiring knowledge of, detecting, or identifying the presence of one or more of such biomarkers in one or more samples from an individual can: a) identify an individual at risk for CRC recurrence; b) identify an individual at risk for CRC recurrence who may benefit from a treatment comprising an anti-cancer therapy; c) be used for selecting a therapy or treatment for an individual at risk for CRC recurrence; d) be used for identifying one or more treatment options for an individual at risk for CRC recurrence; e) be used to predict survival (e.g., length of survival) of an individual having a CRC; f) be used to predict survival (e.g., length of survival) of an individual having a CRC treated with a treatment comprising an anti-cancer therapy; g) be used to predict likelihood of recurrence of a CRC; h) be used in methods for treating or delaying progression of a CRC; i) be used in monitoring, evaluating or screening an individual for CRC recurrence risk; j) be used in monitoring recurrence of a CRC in an individual; k) identify, classify or predict an individual as being at risk for CRC recurrence; l) identify, classify or predict an individual as being at greater risk for CRC recurrence, for example, as compared to an individual with a CRC that does not comprise the one or more biomarkers; m) identify, classify or predict an individual as being at risk for CRC recurrence and as likely to benefit from a treatment comprising an anti-cancer therapy; n) identify, classify or predict an individual as being at risk for CRC recurrence and as likely to benefit from a chemotherapy; o) identify, classify or predict an individual as being at risk for CRC recurrence, and (i) as a candidate to receive a treatment comprising an anti-cancer therapy, or (ii) as likely to respond to a treatment that comprises an anti-cancer therapy; p) identify, classify or predict an individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identify, classify or predict an individual as being at risk for early CRC recurrence; r) identify, classify or predict an individual as being at greater risk for early CRC recurrence, as compared to an individual with a CRC that does not comprise the one or more biomarkers; s) identify, classify or predict an individual as being at risk for CRC recurrence within about one year or less from the time of initial diagnosis of the CRC; and/or t) identify, classify or predict an individual as being at greater risk for CRC recurrence within about one year or less from the time of initial diagnosis of the CRC, as compared to an individual with a CRC that does not comprise the one or more biomarkers.

### A. Biomarkers of CRC Recurrence

Certain aspects of the present disclosure relate to biomarkers that can be used to assess colorectal cancer (CRC) recurrence risk in an individual. In some embodiments, such biomarkers include one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof. In some embodiments, the biomarkers include one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, or SPEN, or any combination thereof. In some embodiments, the biomarkers include one or more, or all, of: (a) an MSI-H status, (b) a high TMB, and (c) a PD-L1 positive status.

In some embodiments, acquiring knowledge of, detecting or identifying the presence of one or more of such biomarkers in one or more samples from an individual, e.g., an individual having a CRC, suspected of having a CRC, being tested for CRC, or being treated for CRC, can be used for one or more, or all, of: a) identifying an individual at risk for CRC recurrence; b) identifying an individual at risk for CRC recurrence who may benefit from a treatment comprising an anti-cancer therapy; c) selecting a therapy or treatment for an individual at risk for CRC recurrence; d) identifying one or more treatment options for an individual at risk for CRC recurrence; e) predicting survival (e.g., length of survival) of an individual having a CRC; f) predicting survival (e.g., length of survival) of an individual having a CRC treated with a treatment comprising an anti-cancer therapy; g) predicting likelihood of recurrence of a CRC; h) treating or delaying progression of a CRC; i) monitoring, evaluating or screening an individual for CRC recurrence risk; j) monitoring recurrence of a CRC in an individual; k) identifying, classifying or predicting an individual as being at risk for CRC recurrence; l) identifying, classifying or predicting an individual as being at greater risk for CRC recurrence, for example, as compared to an individual with a CRC that does not comprise the one or more biomarkers; m) identifying, classifying or predicting an individual as being at risk for CRC recurrence and as likely to benefit from a treatment comprising an anti-cancer therapy; n) identifying, classifying or predicting an individual as being at risk for CRC recurrence and as likely to benefit from a chemotherapy; o) identifying, classifying or predicting an individual as being at risk for CRC recurrence, and (i) as a candidate to receive a treatment comprising an anti-cancer therapy, or (ii) as likely to respond to a treatment that comprises an anti-cancer therapy; p) identifying, classifying or predicting an individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identifying, classifying or predicting an individual as being at risk for early CRC recurrence; r) identifying, classifying or predicting an individual as being at greater risk for early CRC recurrence, as compared to an individual with a CRC that does not comprise the one or more biomarkers; s) identifying, classifying or predicting an individual as being at risk for CRC recurrence within about one year or less from the time of initial diagnosis of the CRC; and/or t) identifying, classifying or predicting an individual as being at greater risk for CRC recurrence within about one year or less from the time of initial diagnosis of the CRC, as compared to an individual with a CRC that does not comprise the one or more biomarkers.

### (i) Microsatellite Instability Status

In some embodiments, the methods provided herein comprise detecting, acquiring knowledge of, or identifying the presence or absence of a microsatellite instability high (MSI-H) status in one or more samples from an individual, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence.

Microsatellite instability (MSI) is a condition of genetic hypermutability that can result from impaired DNA mismatch repair (MMR). The presence of MSI can indicate that MMR is not functioning normally. MSI has been associated with mutations in any one of five human MMR genes: MSH2, MLH1, MSH6, PMS2, and PMS1. Cancers with a high degree of MSI are termed MSI high, or MSI-H, e.g., as described below.

MSI status, such as MSI-H, may be assessed using any suitable method known in the art. For example, MSI may be measured using sequencing (e.g., a massively parallel sequencing (MPS) technique, whole genome sequencing (WGS), whole exome sequencing, targeted sequencing, direct sequencing, or a Sanger sequencing technique), next generation sequencing (see, e.g., Hempelmann et al., J Immunother Cancer (2018) 6(1):29), a PCR-based amplification technique, a non-PCR amplification technique, an isothermal amplification technique, Fluorescent multiplex PCR, capillary electrophoresis (see, e.g., Arulananda et al., J Thorac Oncol (2018) 13(10):1588-94), immunohistochemistry (see, e.g., Cheah et al., Malays J Pathol (2019) 41(2):91-100), or single-molecule molecular inversion probes (smMIPs, see, e.g., Waalkes et al., Clin Chem (2018) 64(6):950-8). In some embodiments, MSI is assessed based on DNA sequencing (e.g., next generation sequencing) of intronic homopolymer repeat loci for length variability. In some embodiments, MSI is assessed based on DNA sequencing (e.g., next generation sequencing) of up to about 114 loci. In some embodiments, MSI is assessed based on DNA sequencing (e.g., next generation sequencing NGS) of about 114 intronic homopolymer repeat loci for length variability. In some embodiments, MSI status is determined as described in Trabucco et al., J Mol Diagn. 2019 November; 21(6):1053-1066.

In some specific embodiments, MSI status is assessed using immunohistochemistry, e.g., based on MLH1, MSH2, MSH6, and PMS2 staining on tumor samples to identify the loss of protein expression that characterizes MMR deficiency as a surrogate for MSI. In other embodiments, MSI status is assessed using PCR to evaluate a panel of microsatellite markers, e.g., a 5-marker panel that includes for example, 2 mononucleotide (BAT25/26) and 3 dinucleotide markers (D2S123, D5S346, and D17S250), to identify instability in the loci. In some embodiments, MSI status is assessed using sequencing, e.g., NGS, to identify length variability, for example in intronic homopolymer repeat loci (e.g., about 114 such loci).

In some embodiments, MSI status is assessed using nucleic acid sequencing data and a per-locus analysis of variant allele frequencies (i.e., the frequencies for variant alleles of altered length) at a plurality of microsatellite loci. Several filters may be used to exclude candidate alleles that are likely to be the result of noise, sequencing errors, or that are germline alleles. The input microsatellite allele sequences are individually categorized as stable or unstable, and an MSI score for the sample is calculated by dividing the number of unstable loci (i.e., loci exhibiting at least one unstable allele) by the total number of loci evaluated for allelic stability (e.g., the total number of loci that met a specified minimum sequencing coverage requirement) to determine the fraction of microsatellite loci that are unstable. A threshold may be applied to the MSI score for classification of the sample as microsatellite instability-high (MSI-H), microsatellite instability-equivocal (MSI-E), or microsatellite stable (MSS). In some specific embodiments, MSI status may be assessed by: receiving, by one or more processors, nucleic acid sequence data for a plurality of microsatellite loci in the sample; identifying, by the one or more processors, a set of microsatellite loci from the plurality of microsatellite loci based on a coverage requirement; applying, by the one or more processors, a set of sequence-based exclusion criteria to the set of microsatellite loci to identify a subset of the set of microsatellite loci; calculating, by the one or more processors, a microsatellite instability (MSI) score for the sample based on the number of microsatellite loci in the set and the number of microsatellite loci in the subset; comparing, by the one or more processors, the MSI score to a threshold (e.g., a first threshold); and if the MSI score is greater than or equal to the threshold, determining an MSI status of high microsatellite instability (MSI-H) for the sample. In some embodiments, an MSI status of high microsatellite instability may be indicative of a deficient DNA mismatch repair mechanism in a tissue of the subject. In some embodiments, applying a set of exclusion criteria to a set of microsatellite loci may comprise “filtering” the set of microsatellite loci, or “removing” microsatellite loci that meet the set of exclusion criteria from the original set of microsatellite loci. In some embodiments, the methods may further comprise comparing the MSI score to a second threshold if the MSI score is less than the first (e.g., the predetermined) threshold; and if the MSI score is less than or equal to the second threshold, determining an MSI status of microsatellite stable (MSS) for the sample; if the MSI score is greater than the second threshold, determining an MSI status of equivocal microsatellite instability (MSI-E) for the sample.

In some instances, the threshold (or first threshold) may be determined by performing a plurality of iterations to obtain a plurality of candidate first threshold values, and averaging the plurality of candidate first threshold values to determine the first threshold. In some instances, each iteration of the (or first) threshold determination process comprises: randomly selecting a subset of a plurality of samples from a plurality of patients; calculating a plurality of MSI scores for the subset; and obtaining a candidate first threshold value that maximizes concordance with determinations of high microsatellite instability obtained using a reference microsatellite instability assay for the subset of the plurality of samples from the plurality of patients. In some instances, for example, the candidate first threshold value is set to a value that maximizes a sum of positive percent agreement (PPA) and negative percent agreement (NPA) with microsatellite instability status results obtained using the reference microsatellite instability assay, while requiring that NPA is greater than a first minimum requirement. In some instances, the second threshold is determined by performing a plurality of iterations to obtain a plurality of candidate second threshold values, and averaging the plurality of candidate second threshold values to determine the second threshold. In some instances, each iteration of the second threshold determination process may comprise randomly selecting a subset of a plurality of samples from a plurality of patients; calculating a plurality of MSI scores for the subset; and obtaining a candidate second threshold value that maximizes concordance with determinations of microsatellite stability obtained using a reference microsatellite instability assay for the subset of the plurality of samples from the plurality of patients. In some instances, the candidate second threshold value is set to a value that maximizes the sum of positive percent agreement (PPA) and negative percent agreement (NPA) with microsatellite instability status results obtained using the reference microsatellite instability assay, while requiring that NPA is greater than a second minimum requirement.

The sequencing-based methods described above to assess MSI status enable the analysis of a large number of microsatellite loci (e.g., hundreds to thousands of individual microsatellite loci)—each comprising mononucleotide, dinucleotide, trinucleotide, or longer repeat sequence motifs—for the presence of variant alleles having altered length. Various exclusion criteria may be applied to the input microsatellite sequence data, for example, to eliminate loci for which the sequencing coverage is inadequate, or to eliminate loci that exhibit alleles that fail to meet a minimum allele frequency requirement, correspond to known germline alleles, correspond to known sequencing errors, and the like, from the analysis, thereby improving the accuracy of the determination of microsatellite instability (MSI) status.

In some embodiments, MSI status is assessed according to the methods described in WO2023287410, which is hereby incorporated by reference in its entirety.

In some embodiments, an MSI-H status is determined if about 1% or more (e.g., any of about 1% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%) of analyzed loci show instability. In some embodiments, an MSI-H status is determined if about 1.24% or more (e.g., any of about 1.24% or more, about 1.5% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%) of analyzed loci show instability.

In some embodiments, an MSI-H status is determined if about 30% or more (e.g., any of about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100% or more) of analyzed loci show instability. In some embodiments, a microsatellite stable (MSS) status is determined if none of the microsatellite markers analyzed show instability. In some embodiments, an MSI-low (MSI-L) status is determined if fewer than 30% (e.g., any of about 29% or less, about 25% or less, about 20% or less, about 15% or less, about 10% or less, about 5% or less, or about 1% or less) of the analyzed loci are unstable. See, e.g., Battaglin et al., Microsatellite instability in colorectal cancer: overview of its clinical significance and novel perspectives. Clin Adv Hematol Oncol. 2018; 16(11):735-745, for a review microsatellite instability in CRC.

In some embodiments, a CRC of the disclosure is, or is determined to be (e.g., according to any method known in the art and/or described herein), microsatellite stable. In some embodiments, a CRC of the disclosure has, or is determined to have (e.g., according to any method known in the art and/or described herein), high microsatellite instability (MSI-H). In some embodiments, a CRC of the disclosure has, or is determined to have (e.g., according to any method known in the art and/or described herein), low microsatellite instability (MSI-L).

In some embodiments, acquiring knowledge of, detecting or identifying the presence of an MSI-H status in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), can be used for one or more, or all, of: a) identifying the individual as being at risk for CRC recurrence; b) identifying the individual as being at risk for CRC recurrence and as one who may benefit from a treatment comprising an anti-cancer therapy; c) selecting a therapy or treatment for the individual; d) identifying one or more treatment options for the individual; e) predicting survival (e.g., length of survival) of the individual; f) predicting survival (e.g., length of survival) of the individual when treated with a treatment comprising an anti-cancer therapy; g) predicting likelihood of recurrence of a CRC in the individual; h) treating or delaying progression of a CRC in the individual; i) monitoring, evaluating or screening the individual for CRC recurrence risk; j) monitoring recurrence of a CRC in the individual; k) identifying, classifying or predicting the individual as being at risk for CRC recurrence; l) identifying, classifying or predicting the individual as being at greater risk for CRC recurrence, for example, as compared to an individual with a CRC that does not comprise the one or more biomarkers; m) identifying, classifying or predicting the individual as being at risk for CRC recurrence and as likely to benefit from a treatment comprising an anti-cancer therapy; n) identifying, classifying or predicting the individual as being at risk for CRC recurrence and as likely to benefit from a chemotherapy; o) identifying, classifying or predicting the individual as being at risk for CRC recurrence, and (i) as a candidate to receive a treatment comprising an anti-cancer therapy, or (ii) as likely to respond to a treatment that comprises an anti-cancer therapy; p) identifying, classifying or predicting the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identifying, classifying or predicting the individual as being at risk for early CRC recurrence; r) identifying, classifying or predicting the individual as being at greater risk for early CRC recurrence, as compared to an individual with a CRC that does not comprise the one or more biomarkers; s) identifying, classifying or predicting the individual as being at risk for CRC recurrence within about one year or less from the time of initial diagnosis of the CRC; and/or t) identifying, classifying or predicting the individual as being at greater risk for CRC recurrence within about one year or less from the time of initial diagnosis of the CRC, as compared to an individual with a CRC that does not comprise the one or more biomarkers.

In some embodiments, MSI status is assessed in sample from an individual, such as a sample described herein. In some embodiments, the sample from the individual comprises fluid, cells, or tissue, e.g., from a liquid biopsy or a tissue biopsy such as a tumor biopsy, e.g., a described in greater detail herein. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

### (ii) Tumor Mutational Burden

In some embodiments, the methods provided herein comprise detecting, acquiring knowledge of, or identifying the presence or absence of high tumor mutational burden (TBM) in one or more samples from an individual, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence.

Tumor mutational burden (TMB) refers to the approximate amount of gene mutations that occurs in a cancer or tumor.

TMB may be measured using any suitable method known in the art. For example, using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, TMB is measured using gene-targeted sequencing, e.g., using a nucleic acid hybridization-capture method, e.g., coupled with sequencing. See, e.g., Fancello et al., J Immunother Cancer (2019) 7:183.

In some embodiments, TMB is measured according to the methods provided in WO2017151524A1, which is hereby incorporated by reference in its entirety. In some embodiments, TMB is measured according to the methods described in Montesion, M., et al., Cancer Discovery (2021) 11(2):282-92.

In some embodiments, TMB is measured according to the methods described in Chalmers et al., Genome Med (2017) 19; 9(1):34. In some embodiments, TMB is assessed as the number of somatic, coding, base substitution, and indel mutations per megabase of genome examined. In some embodiments, all base substitutions and indels in the coding regions of targeted genes, including synonymous alterations, are counted. In some embodiments, assessment of TMB further comprises filtering/counting of the base substitutions and indels, comprising one or more, or all, of the following steps: (a) synonymous mutations are counted in order to reduce sampling noise; (b) non-coding alterations are not counted; (c) alterations listed as known somatic alterations in COSMIC (see, e.g., cancer.sanger.ac.uk/cosmic) and truncations in tumor suppressor genes are not counted; (d) alterations predicted to be germline by a somatic-germline zygosity algorithm (Sun et al., Cancer Res. 2014; 74(19S):1893) are not counted; (e) alterations that are recurrently predicted to be germline are not counted; (f) known germline alterations in dbSNP (see, e.g., www.ncbi.nlm.nih.gov/snp/) are not counted; and (g) germline alterations occurring with two or more counts in the ExAC database (Lek et al., Nature. 2016; 536:285-91.) are not counted. In some embodiments, to calculate TMB per megabase, the total number of mutations counted (e.g., as described above) is divided by the size of the coding region of the targeted territory. In some embodiments, the nonparametric Mann-Whitney U test is used to test for significance in difference of means between two populations.

In some embodiments, TMB is assessed based on the number of non-driver somatic coding mutations/megabase (mut/Mb) of genome sequenced.

In some embodiments, TMB is measured in the sample by whole exome sequencing. In some embodiments, TMB is measured in the sample using next-generation sequencing. In some embodiments, TMB is measured in the sample using whole genome sequencing. In some embodiments, TMB is measured in the sample by gene-targeted sequencing. In some embodiments, TMB is measured on between about 0.7 Mb and about 1.3 Mb of sequenced DNA. In some embodiments, TMB is measured on any of about 0.7 Mb, about 0.75 Mb, about 0.79 Mb, about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, TMB is measured on about 0.79 Mb of sequenced DNA. In some embodiments, TMB is measured on between about 0.83 Mb and about 1.14 Mb of sequenced DNA. In some embodiments, TMB is measured on about 0.8 Mb of sequenced DNA. In some embodiments, TMB is measured on between about 0.83 Mb and about 1.14 Mb of sequenced DNA. In some embodiments, TMB is measured on between about 0.83 Mb and about 1.1 Mb of sequenced DNA. In some embodiments, TMB is measured on up to about 1.24 Mb of sequenced DNA. In some embodiments, TMB is measured on up to about 1.1 Mb of sequenced DNA.

In some embodiments, a high TMB comprises a TMB of at least about 5 mut/Mb. In some embodiments, a high TMB comprises a TMB of at least about 10 mut/Mb. In some embodiments, a high TMB comprises a TMB of at least about 20 mut/Mb. In some embodiments, a high TMB comprises a TMB of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mu/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, a high TMB comprises a TMB of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, a high TMB comprises a TMB of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, measuring TMB comprises assessing mutations in a sample derived from a cancer in an individual. In some embodiments, measuring TMB comprises assessing mutations in a sample derived from a cancer in an individual and in a matched normal sample, e.g., a sample from the individual derived from a tissue or other source that is free of the cancer.

In some embodiments, TMB is obtained from a plurality of sequence reads, e.g., a plurality of sequence reads obtained by sequencing nucleic acids corresponding to at least a portion of a genome (such as from an enriched or unenriched sample), e.g., according to any sequencing method known in the art or described herein. In some embodiments, TMB is determined based on the number of non-driver somatic coding mutations per megabase of genome sequenced.

In some embodiments, acquiring knowledge of, detecting or identifying the presence of a high TMB status in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), can be used for one or more, or all, of: a) identifying the individual as being at risk for CRC recurrence; b) identifying the individual as being at risk for CRC recurrence and as one who may benefit from a treatment comprising an anti-cancer therapy; c) selecting a therapy or treatment for the individual; d) identifying one or more treatment options for the individual; e) predicting survival (e.g., length of survival) of the individual; f) predicting survival (e.g., length of survival) of the individual when treated with a treatment comprising an anti-cancer therapy; g) predicting likelihood of recurrence of a CRC in the individual; h) treating or delaying progression of a CRC in the individual; i) monitoring, evaluating or screening the individual for CRC recurrence risk; j) monitoring recurrence of a CRC in the individual; k) identifying, classifying or predicting the individual as being at risk for CRC recurrence; l) identifying, classifying or predicting the individual as being at greater risk for CRC recurrence, for example, as compared to an individual with a CRC that does not comprise the one or more biomarkers; m) identifying, classifying or predicting the individual as being at risk for CRC recurrence and as likely to benefit from a treatment comprising an anti-cancer therapy; n) identifying, classifying or predicting the individual as being at risk for CRC recurrence and as likely to benefit from a chemotherapy; o) identifying, classifying or predicting the individual as being at risk for CRC recurrence, and (i) as a candidate to receive a treatment comprising an anti-cancer therapy, or (ii) as likely to respond to a treatment that comprises an anti-cancer therapy; p) identifying, classifying or predicting the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identifying, classifying or predicting the individual as being at risk for early CRC recurrence; r) identifying, classifying or predicting the individual as being at greater risk for early CRC recurrence, as compared to an individual with a CRC that does not comprise the one or more biomarkers; s) identifying, classifying or predicting the individual as being at risk for CRC recurrence within about one year or less from the time of initial diagnosis of the CRC; and/or t) identifying, classifying or predicting the individual as being at greater risk for CRC recurrence within about one year or less from the time of initial diagnosis of the CRC, as compared to an individual with a CRC that does not comprise the one or more biomarkers.

In some embodiments, TMB is assessed in sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

(iii) PD-L1 Expression

In some embodiments, the methods provided herein comprise detecting, acquiring knowledge of, or identifying the presence or absence of a PD-L1 positive status in one or more samples from an individual, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, PD-L1 expression is measured by immunohistochemistry using commercially available antibody clones 22C3 (Dako/Agilent) or SP142 (Ventana), e.g., according to methods known in the art and/or described herein.

In some embodiments, a PD-L1 positive status is determined if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a PD-L1 positive status is determined if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells. In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells.

In some embodiments of any of the methods provided herein, a sample from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) is determined to be PD-L1-negative if less than 1% of tumor cells in the sample express PD-L1. In some embodiments of any of the methods provided herein, a sample from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) is determined to be PD-L1 positive if at least about 1% of tumor cells in the sample express PD-L1.

In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs) using a VENTANA PD-L1 assay (SP142). Additional information about the VENTANA SP142 assay may be found in the website: www[dot]accessdata[dot]fda[dot]gov/cdrh_docs/pdf16/P160002c.pdf. In some embodiments, a PD-L1 positive status is determined if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein (e.g., are positive for PD-L1 protein). In some embodiments, a PD-L1 positive status is determined if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor area, e.g., in a sample, is occupied by PD-L1-expressing tumor-infiltrating immune cells (ICs) and/or tumor cells (TCs) of any intensity.

In some embodiments, the level of PD-L1 protein expression is assessed based on a tumor proportion score (TPS). The TPS is the percentage of tumor cells showing partial or complete PD-L1 membrane staining (e.g., at a ≥1+ intensity on a 0, 1+, 2+, and 3 scale) relative to all tumor cells present in the sample. In some embodiments, the TPS is calculated as: the number of PD-L1-positive tumor cells/Total number of PD-L1-positive tumor cells+Total number of PD-L1-negative tumor cells. A PD-L1 low positive status refers to a TPS of between 1% and 49%, PD-L1 high positive status refers to a TPS of 50% or greater, and a PD-L1 negative status refers to a TPS of less than 1%. In some embodiments, a PD-L1 positive status includes a PD-L1 low positive status or a PD-L1 high positive status. In some embodiments, a PD-L1 positive status comprises a TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%. In some embodiments, a PD-L1 low positive status comprises a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%. In some embodiments, a PD-L1 high positive status comprises a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In some embodiments, a PD-L1 negative status comprises a TPS of less than 1%. In some embodiments, the TPS is determined using a DAKO 22C3 assay. Additional information about the DAKO 22C3 assay and the TPS score may be found, e.g., in the website: www[dot]agilent[dot]com/cs/library/usermanuals/public/29158_pd-l1-ihc-22C3-pharmdx-nsclc-interpretation-manual.pdf.

In some embodiments, PD-L1 expression is assessed based on a combined positive score (CPS). The CPS refers to the number of PD-L1 staining cells (e.g., tumor cells, lymphocytes, or macrophages) divided by the total number of viable tumor cells, and multiplied by 100. See, e.g., www[dot]agilent[dot]com/en/product/pharmdx/pd-l1-ihc-22c3-pharmdx-overview#pink3. In some embodiments, a PD-L1 positive status comprises a CPS of at least about 1, at least about 2, at least about 3, at least about 4, at least about 5, at least about 6, at least about 7, at least about 8, at least about 9, or at least about 10. In some embodiments, a cancer of the disclosure, e.g., a CRC, has high PD-L1 expression, e.g., with a CPS of at least about 1, such as between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, or about 100. In some embodiments, PD-L1 expression based on CPS is assessed using a DAKO 22C3 assay. Additional information about the DAKO 22C3 assay and the CPS may be found, e.g., in the websites: www[dot]agilent[dot]com/en/product/pharmdx/pd-l1-ihc-22c3-pharmdx-overview#pink3; www[dot]agilent[dot]com/cs/library/usermanuals/public/29171_22C3-ihc-pharmdx-interpretation-manual-eu.pdf, www[dot]agilent[dot]com/cs/library/usermanuals/public/13350a_eu_urothelial_carcinoma_inter pretation_manual_r3v9_fin_150_single.pdf.pdf; and www[dot]agilent[dot]com/cs/library/usermanuals/public/29314_22c3_pharmDx_hnscc_interpret ation_manual_us.pdf.

In some embodiments of any of the methods provided herein, PD-L1 expression is assessed using a companion diagnostic device, e.g., as provided in www[dot]fda[dot]gov/medical-devices/in-vitro-diagnostics/list-cleared-or-approved-companion-diagnostic-devices-in-vitro-and-imaging-tools.

In some embodiments, acquiring knowledge of, detecting or identifying the presence of a PD-L1 positive status in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), can be used for one or more, or all, of: a) identifying the individual as being at risk for CRC recurrence; b) identifying the individual as being at risk for CRC recurrence and as one who may benefit from a treatment comprising an anti-cancer therapy; c) selecting a therapy or treatment for the individual; d) identifying one or more treatment options for the individual; e) predicting survival (e.g., length of survival) of the individual; f) predicting survival (e.g., length of survival) of the individual when treated with a treatment comprising an anti-cancer therapy; g) predicting likelihood of recurrence of a CRC in the individual; h) treating or delaying progression of a CRC in the individual; i) monitoring, evaluating or screening the individual for CRC recurrence risk; j) monitoring recurrence of a CRC in the individual; k) identifying, classifying or predicting the individual as being at risk for CRC recurrence; l) identifying, classifying or predicting the individual as being at greater risk for CRC recurrence, for example, as compared to an individual with a CRC that does not comprise the one or more biomarkers; m) identifying, classifying or predicting the individual as being at risk for CRC recurrence and as likely to benefit from a treatment comprising an anti-cancer therapy; n) identifying, classifying or predicting the individual as being at risk for CRC recurrence and as likely to benefit from a chemotherapy; o) identifying, classifying or predicting the individual as being at risk for CRC recurrence, and (i) as a candidate to receive a treatment comprising an anti-cancer therapy, or (ii) as likely to respond to a treatment that comprises an anti-cancer therapy; p) identifying, classifying or predicting the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identifying, classifying or predicting the individual as being at risk for early CRC recurrence; r) identifying, classifying or predicting the individual as being at greater risk for early CRC recurrence, as compared to an individual with a CRC that does not comprise the one or more biomarkers; s) identifying, classifying or predicting the individual as being at risk for CRC recurrence within about one year or less from the time of initial diagnosis of the CRC; and/or t) identifying, classifying or predicting the individual as being at greater risk for CRC recurrence within about one year or less from the time of initial diagnosis of the CRC, as compared to an individual with a CRC that does not comprise the one or more biomarkers.

In some embodiments, the level of PD-L1 protein and/or PD-L1 mRNA is assessed in a sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from a cancer or the disclosure, e.g., a CRC.

### (iv) Gene Alterations

In some embodiments, the methods provided herein comprise detecting, acquiring knowledge of, or identifying the presence or absence of an alteration in one or more genes in one or more samples from an individual, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence.

In some embodiments, the alteration in the one or more genes is a point mutation, an insertion-deletion (indel), an in-frame deletion of one or more codons, an intragenic deletion, an intragenic insertion, a deletion of a full gene, an inversion, an interchromosomal or intrachromosomal translocation, a tandem duplication, a gene fusion, a genomic rearrangement, a splice site mutation, and/or a gene amplification or duplication.

In some embodiments, the one or more genes comprise one or more of BRAF, PTEN, RNF43, ASXL1, CREBBP, MLL2, BCORL1, ATR, SPEN, BRCA1, BRCA2, or MSH6, or any combination thereof. In some embodiments, the one or more genes comprise one or more of BRAF, PTEN, RNF43, ASXL1, CREBBP, MLL2, BCORL1, ATR, or SPEN, or any combination thereof.

In some embodiments, the alteration in BRAF is a V600E, D594G, G469A, N581S, G466V, K483E, L485F, L485S, or T241M alteration, or any combination thereof. As used herein “BRAF” refers to a gene encoding a BRAF mRNA or polypeptide. The BRAF gene encodes the B-Raf serine/threonine kinase protein. BRAF is also known as NS7, B-raf, BRAF1, RAFB1, B-RAF1, BRAF-1, 94 kDa B-raf protein, B-Raf proto-oncogene serine/threonine-protein kinase (p94), murine sarcoma viral (v-raf) oncogene homolog B1, proto-oncogene B-Raf, v-raf murine sarcoma viral oncogene homolog B, and v-raf murine sarcoma viral oncogene homolog B1. In some embodiments, a BRAF gene is a human BRAF gene. An exemplary BRAF gene is represented by NCBI Gene ID No. 673. An exemplary BRAF mRNA sequence is represented by NCBI Ref. Seq. NM_004333. An exemplary amino acid sequence of a BRAF polypeptide is represented by NCBI Ref. Seq. NP_004324.

In some embodiments, the alteration in PTEN is a K267fs*9, N323fs*21, R233*, R130*, R130Q, E299*, R173H, T319fs*1, C136Y, C250fs*2, D24fs*20, E157fs*23, E242fs*9, F90fs*9, H93R, I33del, K164fs*3, K330*, L247*, L325P, L57fs*6, N323fs*2, N340fs*3, N63fs*11, P95L, Q171*, Q219*, Q245*, Q261*, R234W, S59*, splice site 209+5G>A, splice site 210−1G>C, splice site 626_634+2delGAACTTGCAGT, splice site 79+1G>A, T131N, or V133I alteration, or any combination thereof. As used herein “PTEN” refers to a gene encoding a PTEN mRNA or polypeptide. The PTEN gene encodes the PTEN phosphatidylinositol 3,4,5-trisphosphate 3-phosphatase and dual-specificity protein phosphatase protein. PTEN is also known as BZS, DEC, CWS1, GLM2, MHAM, TEP1, MMAC1, PTEN1, 10q23del, PTENbeta, MMAC1 phosphatase and tensin homolog, PTENepsilon, mitochondrial PTENalpha, mitochondrial phosphatase and tensin protein alpha, mutated in multiple advanced cancers 1, phosphatase and tensin-like protein, and protein tyrosine phosphatase. In some embodiments, a PTEN gene is a human PTEN gene. An exemplary PTEN gene is represented by NCBI Gene ID No. 5728. An exemplary PTEN mRNA sequence is represented by NCBI Ref. Seq. NM_000314. An exemplary amino acid sequence of a PTEN polypeptide is represented by NCBI Ref. Seq. NP_000305.

In some embodiments, the alteration in RNF43 is a G659fs*41, R117fs*41, R225fs*194, R132*, R145*, R330*, Y332*, A193fs*6, A273fs*147, A78T, E258fs*162, G257fs*162, K181fs*4, L311fs*132, M1I, P660fs*87, Q153*, Q233*, Q426*, Q426fs*77, Q8*, R225fs*195, R337*, splice site 375+1G>A, V271fs*11, V299fs*120, V479fs*25, W159*, W302*, Y248*, or Y332fs*110V alteration, or any combination thereof. As used herein “RNF43” refers to a gene encoding a RNF43 mRNA or polypeptide. The RNF43 gene encodes the RNF43 ring finger protein. RNF43 is also known as URCC, SSPCS, RNF124, E3 ubiquitin-protein ligase RNF43 and RING-type E3 ubiquitin transferase RNF43. An exemplary RNF43 gene is represented by NCBI Gene ID No. 54894. An exemplary RNF43 mRNA sequence is represented by NCBI Ref. Seq. NM_017763. An exemplary amino acid sequence of an RNF43 polypeptide is represented by NCBI Ref. Seq. NP_001292473.

In some embodiments, the alteration in ASXL1 is a G645fs*58, G646fs*12, R693*, A627fs*8, E41K, E676*, G646fs*58, L983fs*8, P1377fs*3, P763fs*12, Q561fs*1, or S892fs*16 alteration, or any combination thereof. As used herein “ASXL1” refers to a gene encoding an ASXL1 mRNA or polypeptide. The ASXL1 gene encodes the ASXL1 polycomb group protein. ASXL1 is also known as MDS, BOPS, ASXL transcriptional regulator 1, additional sex combs like 1 transcriptional regulator, additional sex combs like transcriptional regulator 1, and putative Polycomb group protein ASXL1. An exemplary ASXL1 gene is represented by NCBI Gene ID No. 171023. An exemplary ASXL1 mRNA sequence is represented by NCBI Ref. Seq. NM_015338. An exemplary amino acid sequence of an ASXL1 polypeptide is represented by NCBI Ref. Seq. NP_056153.

In some embodiments, the alteration in CREBBP is a I1084fs*15, P1423fs*36, A1824T, Q1209fs*25, G1145fs*23, R714H, I1084fs*3, K668fs*27, L555fs*7, P2094L, P937fs*61, Q278*, Q911*, R1446H, S801*, splice site 3836+1G>A, or Y1503H alteration, or any combination thereof. As used herein “CREBBP” refers to a gene encoding a CREBBP mRNA or polypeptide. The CREBBP gene encodes the CREBBP CREB-binding protein. CREBBP is also known as CBP, KAT3A, MKHK1, RSTS, RSTS1, histone lysine acetyltransferase CREBBP and protein-lysine acetyltransferase CREBBP. An exemplary CREBBP gene is represented by NCBI Gene ID No. 1387. An exemplary CREBBP mRNA sequence is represented by NCBI Ref. Seq. NM_004380. An exemplary amino acid sequence of a CREBBP polypeptide is represented by NCBI Ref. Seq. NP_004371.

In some embodiments, the alteration in MLL2 is a P2354fs*30 9, G1235fs*95, P647fs*283, T382fs*20, A2205fs*59, C2436fs*49, C346fs*17, D2769N, E2962fs*42, F1790fs*12, G2265fs*21, H77fs*53, I977fs*23, K1686fs*36, K304fs*30, L1020fs*36, L5183fs*16, P1460fs*46, P2206fs*58, P367fs*35, P4380fs*4, P444fs*2, P4968fs*27, P506fs*424, P583fs*347, P648fs*2, Q1377R, Q3811fs*201, R1252*, R1687fs*4, R2771*, R2830*, R4238C, R4904*, R5048H, R5282*, R755fs*3, S1107fs*12, S1684T, S2910fs*32, S4507fs*12, S4789fs*27, splice site 14644−1G>T, splice site 16413−2A>G, T209fs*11, V1244fs*86, V1670fs*52, or V4799M alteration, or any combination thereof. As used herein “MLL2” refers to a gene encoding a MLL2 mRNA or polypeptide. The MLL2 gene encodes the MLL2 histone-lysine N-methyltransferase 2D protein. In some embodiments, an MLL2 gene is a human MLL2 gene. An exemplary MLL2 gene is represented by NCBI Gene ID No. 8085. An exemplary MLL2 mRNA sequence is represented by NCBI Ref. Seq. NM_003482. An exemplary amino acid sequence of an MLL2 polypeptide is represented by NCBI Ref. Seq. NP_003473.

In some embodiments, the alteration in BCORL1 is a P1681fs*20, A1166fs*56, A971fs*4, E1655*, E619*, G1682fs*4, K1207N, P323fs*95, Q1001fs*49, R1297*, R1299*, R1420*, or W1105* alteration, or any combination thereof. As used herein “BCORL1” refers to a gene encoding a BCORL1 mRNA or polypeptide. The BCORL1 gene encodes the BCORL1 BCL-6 corepressor-like protein 1. BCORL1 is also known as SHUVER, BCoR-L1, CXorf10, BCoR-like protein 1. In some embodiments, a BCORL1 gene is a human BCORL1 gene. An exemplary BCORL1 gene is represented by NCBI Gene ID No. 63035. An exemplary BCORL1 mRNA sequence is represented by NCBI Ref. Seq. NM_001184772. An exemplary amino acid sequence of a BCORL1 polypeptide is represented by NCBI Ref. Seq. NP_001171701.

In some embodiments, the alteration in ATR is a I774fs*5, F1091fs*28, F1134fs*6, I774fs*3, E2579*, F2168*, I691fs*5, K446fs*11, K773fs*3, R2001*, R223fs*1, R2547*, or W1591* alteration, or any combination thereof. As used herein “ATR” refers to a gene encoding an ATR mRNA or polypeptide. The ATR gene encodes the ATR serine/threonine-protein kinase protein. ATR is also known as FRP1, MEC1, SCKL, FCTCS, SCKL1, ATR FRAP-related protein-1, MEC1 mitosis entry checkpoint 1 homolog, and ataxia telangiectasia and Rad3-related protein. In some embodiments, an ATR gene is a human ATR gene. An exemplary ATR gene is represented by NCBI Gene ID No. 545. An exemplary ATR mRNA sequence is represented by NCBI Ref. Seq. NM_001184. An exemplary amino acid sequence of an ATR polypeptide is represented by NCBI Ref. Seq. NP_001175.

In some embodiments, the alteration in SPEN is a A2105fs*33, R806fs*14, A2105fs*18, H2985fs*199, I1052fs*40, I577fs*37, N2002fs*20, P2495fs*4, P2839fs*50, P3631fs*3, Q253fs*109, R1936*, R2332H, or V1294fs*7 alteration, or any combination thereof. As used herein “SPEN” refers to a gene encoding a SPEN mRNA or polypeptide. The SPEN gene encodes the SPEN Msx2-interacting protein. SPEN is also known as MINT, SHARP, RATARS, RBM15C, HIAA0929, Msx2 interacting nuclear target (MINT) homolog, SMART/HDAC1-associated repressor protein, nuclear receptor transcription cofactor, and SPEN homolog transcriptional regulator. In some embodiments, a SPEN gene is a human SPEN gene. An exemplary SPEN gene is represented by NCBI Gene ID No. 23013. An exemplary SPEN mRNA sequence is represented by NCBI Ref. Seq. NM_015001. An exemplary amino acid sequence of a SPEN polypeptide is represented by NCBI Ref. Seq. NP_055816.

In some embodiments, the alteration in BRCA1 is a K654fs*47, Q1756fs*74, Q74*, R1203*, S324fs*16, splice site 4185+2_4185+22>A, or splice site 442−2A>G alteration, or any combination thereof. As used herein “BRCA1” refers to a gene encoding a BRCA1 mRNA or polypeptide. The BRCA1 gene encodes the BRCA1 breast cancer type 1 susceptibility protein. BRCA1 is also known as IRIS, PSCP, BRCA1, BRCC1, FANCS, PNCA4; RNF53, BROVCA1, PPP1R53, BRCA1/BRCA2-containing complex subunit 1, Fanconi anemia complementation group S, RING finger protein 53, breast and ovarian cancer susceptibility protein 1, early onset breast cancer 1, and protein phosphatase 1 regulatory subunit 53. In some embodiments, a BRCA1 gene is a human BRCA1 gene. An exemplary BRCA1 gene is represented by NCBI Gene ID No. 672. An exemplary BRCA1 mRNA sequence is represented by NCBI Ref. Seq. NM_007294. An exemplary amino acid sequence of a BRCA1 polypeptide is represented by NCBI Ref. Seq. NP_009225.

In some embodiments, the alteration in BRCA2 is a E2981K, E2981fs*7, N1784fs*7, R2842C, T3033fs*29, A1237fs*2, C3233fs*15, D252fs*24, E2144*, E3316fs*2, E597*, E866*, M2393fs*19, N1287fs*6, N1784fs*3, N2189fs*2, R2034H, R2318*, T3085fs*26, V1862fs*1, or W2830* alteration, or any combination thereof. As used herein “BRCA2” refers to a gene encoding a BRCA2 mRNA or polypeptide. The BRCA2 gene encodes the BRCA2 breast cancer type 2 susceptibility protein. BRCA2 is also known as FAD, FACD, FAD1, GLM3, BRCC2, FANCD, PNCA2, FANCD1, XRCC11, BROVCA2, BRCA1/BRCA2-containing complex subunit 2, DNA repair-associated BRCA2, Fanconi anemia group D1 protein, early onset breast and ovarian cancer susceptibility gene, breast and ovarian cancer susceptibility protein 2, breast cancer 2 tumor suppressor, breast cancer 2 early onset, mutant BRCA2, and mutant DNA repair-associated protein 2. In some embodiments, a BRCA2 gene is a human BRCA2 gene. An exemplary BRCA2 gene is represented by NCBI Gene ID No. 675. An exemplary BRCA2 mRNA sequence is represented by NCBI Ref. Seq. NM_000059. An exemplary amino acid sequence of a BRCA2 polypeptide is represented by NCBI Ref. Seq. NP_000050.

In some embodiments, the alteration in MSH6 is a F1088fs*5, F1088fs*2, F1088fs*3, E946*, A1236fs*4, A1320fs*5, C694fs*4, K1140fs*24, K247fs*32, L1356fs*1, R240*, R248fs*8, R298*, or R361H alteration, or any combination thereof. As used herein “MSH6” refers to a gene encoding a MSH6 mRNA or polypeptide. The MSH6 gene encodes the MSH6 DNA mismatch repair protein. MSH6 is also known as GTBP, HSAP, p160, GTMBP, MSH-6, HNPCC5, LYNCH5, MMRCS3, G/T mismatch-binding protein, mutS protein homolog 6, mutS-alpha 160 kDa subunit, mutS-like protein 6, and sperm-associated protein. In some embodiments, a MSH6 gene is a human MSH6 gene. An exemplary MSH6 gene is represented by NCBI Gene ID No. 2956. An exemplary MSH6 mRNA sequence is represented by NCBI Ref. Seq. NM_000179. An exemplary amino acid sequence of an MSH6 polypeptide is represented by NCBI Ref. Seq. NP_000170.

An alteration in one or more genes as described herein may be detected using any suitable method known in the art. For example, in some embodiments, an alteration in a gene of the disclosure may be detected by sequencing part or all of the gene by next-generation or other sequencing of DNA, RNA, or cDNA. In some embodiments, an alteration in a gene of the disclosure may be detected by PCR amplification of DNA, RNA, or cDNA. In some embodiments, an alteration in a gene of the disclosure may be detected by in situ hybridization, e.g., using fluorescence in situ hybridization (FISH). In some embodiments, an alteration in a gene of the disclosure may be detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Additional exemplary and non-limiting methods for detecting an alteration in a gene of the disclosure are provided below.

In some embodiments, an alteration in a gene of the disclosure may be detected using a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect an alteration in a gene of the disclosure, are described in U.S. Pat. No. 9,340,830 and in WO2012092426A1, which are hereby incorporated by reference in their entirety. In some embodiments, an alteration in a gene of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, an alteration in a gene of the disclosure is detected using an in situ hybridization method, such as a fluorescence in situ hybridization (FISH) method. In some embodiments, FISH analysis is used to identify a chromosomal rearrangement resulting in an alteration in a gene of the disclosure. In some embodiments, FISH analysis is used to identify an RNA molecule comprising or encoding an alteration in a gene of the disclosure. Methods for performing FISH are known in the art and can be used in nearly any type of tissue. In FISH analysis, nucleic acid probes which are detectably labeled, e.g., fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art. Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

In some embodiments, an alteration in a gene of the disclosure is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, an alteration in a gene of the disclosure is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers. The presence of an alteration in a gene of the disclosure in the sample can be determined, for example, based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green. Other amplification methods suitable for use according to the methods provided herein include, e.g., ligase chain reaction (LCR), transcription amplification, self-sustained sequence replication, dot PCR, and linker adapter PCR.

In some embodiments, an alteration in a gene of the disclosure is detected using a sequencing method. Any method of sequencing known in the art can be used to detect an alteration in a gene of the disclosure. Exemplary sequencing methods that may be used include those based on techniques developed by Maxam and Gilbert or Sanger. Automated sequencing procedures may also be used, e.g., including sequencing by mass spectrometry.

In some embodiments, an alteration in a gene of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, an alteration in a gene of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect an alteration in a gene of the disclosure include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect an alteration in a gene of the disclosure include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), wherein the plurality of nucleic acid molecules comprises nucleic acid molecules corresponding to a gene of the disclosure; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to a gene of the disclosure; (f) analyzing the plurality of sequence reads; and (g) based on the analysis, detecting the presence or absence of an alteration in a gene of the disclosure in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to a gene of the disclosure and/or an alteration in a gene of the disclosure. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a gene of the disclosure in said library to produce an enriched sample; (e) sequencing the enriched sample, thereby producing a plurality of sequence reads; (f) analyzing the plurality of sequence reads for the presence of an alteration in a gene of the disclosure; (g) detecting, based on the analyzing step, the presence or absence of an alteration in a gene of the disclosure in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods provided herein, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments of any of the methods provided herein, the methods comprise selectively enriching for one or more nucleic acids in a sample comprising nucleotide sequences corresponding to a gene of the disclosure. In some embodiments, selectively enriching comprises: (a) combining one or more bait molecules with a sequencing library, thereby hybridizing the one or more bait molecules to one or more nucleic acid molecules comprising nucleotide sequences corresponding to a gene of the disclosure and producing nucleic acid hybrids; and (b) isolating the nucleic acid hybrids to produce an enriched sample. In other embodiments, the selectively enriching comprises: (a) combining one or more bait molecules with a sample, thereby hybridizing the one or more bait molecules to one or more nucleic acids in the sample comprising nucleotide sequences corresponding to a gene of the disclosure and producing nucleic acid hybrids; and (b) isolating the nucleic acid hybrids to produce an enriched sample. In other embodiments, the selectively enriching comprises amplifying one or more nucleic acids comprising nucleotide sequences corresponding to a gene of the disclosure, e.g., using a polymerase chain reaction (PCR) amplification technique, a non-PCR amplification technique, or an isothermal amplification technique, to produce an enriched sample. In other embodiments, nucleic acid molecules comprising nucleotide sequences corresponding to a gene of the disclosure are captured from amplified nucleic acid molecules by hybridization to one or more bait molecules. In some embodiments, the methods further comprise sequencing the enriched sample or the captured nucleic acid molecules. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing is performed using a sequencer, optionally a next generation sequencer.

In some embodiments of any of the methods provided herein, the methods further comprise analyzing sequence data (e.g., obtained from sequencing as described above), for the presence or absence of an alteration in one or more genes of the disclosure. In some embodiments, the presence or absence of the alteration in one or more genes of the disclosure is detected using any suitable method known in the art, e.g., as described in Frampton et al., (2013) Nat Biotechnol, 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol, 31:1023-1031. In some embodiments, insertion/deletion (indel) alterations are detected using any suitable method, such as de novo local assembly, e.g., using the de Bruijn approach, see, e.g., Compeau et al., Nat Biotechnol (2011) 29:987-991 and Frampton et al., (2013) Nat Biotechnol, 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation).

In some embodiments, acquiring knowledge of, detecting or identifying the presence of an alteration in one or more genes of the disclosure in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence), can be used for one or more, or all, of: a) identifying the individual as being at risk for CRC recurrence; b) identifying the individual as being at risk for CRC recurrence and as one who may benefit from a treatment comprising an anti-cancer therapy; c) selecting a therapy or treatment for the individual; d) identifying one or more treatment options for the individual; e) predicting survival (e.g., length of survival) of the individual; f) predicting survival (e.g., length of survival) of the individual when treated with a treatment comprising an anti-cancer therapy; g) predicting likelihood of recurrence of a CRC in the individual; h) treating or delaying progression of a CRC in the individual; i) monitoring, evaluating or screening the individual for CRC recurrence risk; j) monitoring recurrence of a CRC in the individual; k) identifying, classifying or predicting the individual as being at risk for CRC recurrence; l) identifying, classifying or predicting the individual as being at greater risk for CRC recurrence, for example, as compared to an individual with a CRC that does not comprise the one or more biomarkers; m) identifying, classifying or predicting the individual as being at risk for CRC recurrence and as likely to benefit from a treatment comprising an anti-cancer therapy; n) identifying, classifying or predicting the individual as being at risk for CRC recurrence and as likely to benefit from a chemotherapy; o) identifying, classifying or predicting the individual as being at risk for CRC recurrence, and (i) as a candidate to receive a treatment comprising an anti-cancer therapy, or (ii) as likely to respond to a treatment that comprises an anti-cancer therapy; p) identifying, classifying or predicting the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; q) identifying, classifying or predicting the individual as being at risk for early CRC recurrence; r) identifying, classifying or predicting the individual as being at greater risk for early CRC recurrence, as compared to an individual with a CRC that does not comprise the one or more biomarkers; s) identifying, classifying or predicting the individual as being at risk for CRC recurrence within about one year or less from the time of initial diagnosis of the CRC; and/or t) identifying, classifying or predicting the individual as being at greater risk for CRC recurrence within about one year or less from the time of initial diagnosis of the CRC, as compared to an individual with a CRC that does not comprise the one or more biomarkers.

An alteration in one or more genes of the disclosure may be detected in any sample described herein. In some embodiments, the sample is obtained from the individual or from the cancer, e.g., the CRC. In some embodiments, the methods further comprise obtaining the sample, e.g., from the individual or from the cancer, e.g., the CRC. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer, e.g., the CRC. In some embodiments, the sample comprises mRNA, DNA, circulating tumor DNA (ctDNA), cell-free DNA, or cell-free RNA from the cancer, e.g., the CRC. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

### B. Colorectal Cancers and Methods Related Thereto

Certain aspects of the present disclosure relate to use of biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) for: identifying an individual at risk for colorectal cancer (CRC) recurrence; identifying an individual at risk for CRC recurrence who may benefit from a treatment comprising an anti-cancer therapy; selecting a therapy or treatment for an individual at risk for CRC recurrence; identifying one or more treatment options for an individual at risk for CRC recurrence; predicting survival (e.g., length of survival) of an individual having a CRC; predicting survival (e.g., length of survival) of an individual having a CRC treated with a treatment comprising an anti-cancer therapy; predicting likelihood of recurrence of a CRC; treating or delaying progression of a CRC; monitoring, evaluating or screening an individual for CRC recurrence risk; and/or monitoring recurrence of a CRC in an individual.

Colorectal cancer (CRC) refers to cancers of the colon or rectum. CRC may also be referred to as colon cancer or rectal cancer. Types of CRC include adenocarcinoma, carcinoid tumor, familial CRC, gastrointestinal stromal tumor (GIST), colorectal lymphoma, squamous cell carcinoma, leiomyosarcoma, and angiosarcoma.

A commonly used staging system for CRC is the American Joint Committee on Cancer (AJCC) TNM system. The TNM system uses the extent of the tumor (T), spread to nearby lymph nodes (N), and spread (metastasis) to distant sites (M) for staging CRC. Staging may be based on pathologic or surgical findings and/or clinical staging. CRCs may be classified as Stage 0, Stage I, Stage II (including Stage IIA-IIC), Stage III (including Stage IIIA-IIIC), and Stage IV (including Stage IVA-IVC). See, for example, www[dot]cancer[dot]org/cancer/types/colon-rectal-cancer/detection-diagnosis-staging/staged.html, for additional information on CRC staging.

In some embodiments, a CRC of the disclosure is an early CRC, such as a CRC in any of Stages I-III. In some embodiments, a CRC of the disclosure is a Stage 0, Stage I, Stage II, Stage III, or Stage IV CRC. In some embodiments, a CRC of the disclosure is a primary CRC or is metastatic. In some embodiments, a CRC of the disclosure comprises one or more KRAS alterations, such as a G12C, G12D, G12V, or G13D amino acid substitution. In some embodiments, a CRC of the disclosure may be either mismatch repair deficient (dMMR), or may not have a mismatch repair deficiency (i.e., is not a dMMR CRC).

In some embodiments, a CRC of the disclosure is a Stage IV CRC. In some embodiments, a Stage IV CRC may comprise one or more alterations in an APC and/or TP53 gene. In some embodiments, the alteration in an APC gene comprises one or more of: R1450*, R876*, splice site 835−8A>G, T1556fs*3, E1309fs*4, R213*, R216*, R564*, R283*, R232*, R1114*, S1465fs*3, Q1367*, R805*, R499*, Q1429*, Q1406*, E1379*, Q1338*, E1309*, E1306*, Y935*, R554*, E941*, Q1378*, V1414fs*1, R302*, E1353*, Q1291*, Y935fs*1, S1356*, E1322*, E1408*, Q1303*, E1397*, P1319fs*2, E1464fs*8, E1295*, E1286*, 1488fs*19, Q1294*, N1455fs*18, S1411fs*4, K534*, G1312*, S1495fs*12, S1344*, R1399fs*9, F1491fs*16, S1415fs*4, S1400*, S1346*, S1315*, Q1328*, E1345*, E1317*, T1493fs*14, S1436fs*37, S1421fs*52, K1182*, D1486fs*21, R332*, F1396fs*19, A1492fs*22, splice site 1548+1G>A, S457*, S1545*, S1465fs*9, R640W, S943*, S1501fs*6, Q188*, Q1477*, Q1244*, P1442fs*31, P1440fs*33, P1439fs*34, K670*, I1580fs*70, E763*, E1554*, W699*, W423*, V1452fs*21, S1415fs*8, S1355fs*19, Q767*, Q236*, L1302fs*3, E893*, E1573*, E1544*, Y1376*, T1556fs*9, T1438fs*35, Q1480*, Q1096*, P1443fs*30, P1424fs*49, M1383fs*3, L1488fs*26, H1490fs*24, E225*, E1538fs*5, E1374*, E1353fs*62, E1309fs*6, C1387*, Y1376fs*9, W1049*, S932*, S811*, S770*, S1539*, S1400fs*1, S1389fs*5, S1355fs*60, S1327*, S1200fs*7, R653K, R1314fs*7, Q901*, Q793*, Q695*, Q264*, Q1228*, Q1065*, P1433fs*40, L1489fs*19, K1310fs*11, I1580fs*69, I1311fs*4, G1288*, F814fs*6, E991*, E403fs*51, E1577fs*73, E1552*, E1536*, E1530*, E1461*, E1353fs*21, E1284*, E1155*, D170fs*4, C1410*, A1351fs*3, Y935fs*19, Y796fs*2, Y1376fs*10, Y1075*, W685*, V452fs*7, T1445fs*28, splice site 835−1G>A, splice site 645+1G>A, splice site 1409−5A>G, splice site 1409−2A>G, splice site 1312+1G>A, S596*, S1581fs*69, S1567*, S1495fs*19, S1355fs*20, S1298fs*7, S1282*, S1272*, R904fs*7, R904fs*12, R405*, R2237*, Q886*, Q789*, Q757*, Q445*, Q260*, Q1477fs*30, Q1469*, Q1444*, or Q1256*. In some embodiments, the alteration in a TP53 gene comprises one or more of: R175H, R282W, R273H, R248Q, R273C, G245S, R213*, R196*, R248W, R306*, R342*, C176F, M237L, C238Y, P152L, Y220C, G266E, C141Y, splice site 375G>A, V173L, V173M, C135F, R158H, W146*, C176Y, I195T, C275Y, G244S, H179R, H179Y, H214R, R249S, R337C, T211I, V172F, V272M, E285K, E286K, P151S, P278S, R273L, splice site 673−1G>A, T125M, V272L, C275F, E294*, F113V, G244D, G245D, K132N, P27fs*17, Q104*, splice site 672+1G>T, splice site 782+1G>A, V122fs*26, Y163C, A161T, C135Y, C242F, G266R, L257P, L257Q, N131fs*27, P151H, P153fs*28, S166*, S215G, S215N, splice site 375+1G>T, splice site 559+1G>A, V216M, V73fs*76, C238F, C242fs*5, C242Y, E171*, E204*, E258G, E271K, G244A, G266*, H178fs*3, H193Y, K132R, L194F, N239fs*9, P250L, P278T, Q165*, R110L, R209fs*6, R213L, R213Q, S127F, S215L, splice site 672G>A, splice site 782+1G>T, splice site 88_96+1delAACGTTCTGG, V274F, Y126C, Y126N, Y205D, Y234C, Y236C, A159V, A276G, C135R, C135W, C141R, C141W, C277F, D259Y, E198*, E258*, E271*, E298*, E51*, F134L, F270C, G244C, G266V, H193L, L130F, L130V, L194P, L35fs*9, MIT, N247L, P191del, Q167*, Q192*, Q317*, R181H, R181P, R249M, S214W, S241fs*6, S261fs*85, S90fs*33, S90fs*59, splice site 375+5G>T, splice site 375G>C, splice site 376−1G>A, splice site 560−1G>A, splice site 560−1G>T, splice site 560−2A>T, splice site 560−3T>G, splice site 673−1G>T, splice site 920−2A>G, splice site 993+1G>A, V157F, V216L, V274A, V274G, W91*, Y163H, Y205H, Y234H, Y236fs*14, Y236H, C124fs*25, C141*, C182fs*65, C229*, C238R, D393fs*78, E180*, E198fs*49, E221*, E224D, E258A, E258K, E285*, E336fs*4, E339*, E349*, F109V, F134C, F134V, or F270L.

In some embodiments, a CRC of the disclosure is a Stage I, Stage II, or Stage III CRC. In some embodiments, a Stage I, Stage II, or Stage III CRC may comprise an alteration in one or more of a BRCA1, BRCA2, MSH6, MLH1, and/or MSH2 gene. In some embodiments, the alteration in a BRCA1 gene comprises one or more of: K339fs*2, K654fs*47, Q1756fs*74, Y655fs*18, A224fs*4, E181*, E577*, E732*, H1686R, K1711fs*3, L1098fs*4, L63F, Q74*, R1203*, R1495M, R1751*, S1457*, S324fs*16, splice site 4185+2_4185+22>A, splice site 442−2A>G, splice site 5277+1G>A, or V340fs*6. In some embodiments, the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*. In some embodiments, the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H. In some embodiments, the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G. In some embodiments, the alteration in a MSH2 gene comprises one or more of: A230fs*16, S233fs*13, R680*, E580*, E480*, Y408*, splice site 943−1G>C, splice site 942+3A>T, R406*, R389*, Q61*, Q574*, Q324*, L634*, L277fs*5, L187R, K449fs*5, I134fs*8, G683R, E86fs*4, E850*, E188*, C778fs*35, or C778fs*34.

In other embodiments, a Stage I, Stage II, or Stage III CRC may comprise an alteration in one or more of a RNF43, MLL2, MSH3, PTCH1, CDK12, ARID1A, ASXL1, MSH6, BCORL1, CTNNB1, MLH1, CIC, MAP3K1, ATR, MSH2, CTCF, JAK1, QKI, CDH1, CASP8, NOTCH3, EP300, BRCA2, MEN1, or BCOR gene, or any combination thereof. In other embodiments, the alteration in a RNF43 gene comprises one or more of: G659fs*41, R117fs*41, R225fs*194, R117fs*8, R145*, P660fs*41, P660fs*87, R371*, R132*, R330*, R337*, A273fs*147, E37fs*11, K181fs*4, S216L, V479fs*25, Y248*, Y332*, A169T, A193fs*6, A78T, C290*, E258fs*162, E318*, E37*, F103fs*20, G257fs*162, G29*, G659fs*87, H352fs*87, I48T, K60fs*2, L311fs*108, L311fs*132, L53fs*1, L82*, L88fs*13, M1I, M55fs*7, N167fs*1, P370fs*49, P715fs*15, P77fs*18, Q153*, Q233*, Q254*, Q283*, Q426*, Q426fs*77, Q6fs*29, Q8*, R113*, R225fs*195, R286W, R49fs*3, S607L, S687fs*13, splice site 375+1G>A, splice site 583−177_592del187, splice site 687+1G>A, splice site 688−1G>A, splice site 850−2A>G, splice site 952+2T>C, T158fs*10, V271fs*11, V271fs*149, V299fs*120, V490fs*12, W13*, W13fs*26, W159*, W165*, W200*, W302*, Y332fs*110, or Y332fs*111. In other embodiments, the alteration in a MLL2 gene comprises one or more of: P2354fs*30, G1235fs*95, P647fs*283, P648fs*2, R4904*, A1390fs*27, Q836fs*94, A2119fs*25, C2436fs*49, C346fs*17, G5182fs*61, H1497fs*30, R2443fs*6, R4238C, R845fs*3, T382fs*20, T4629fs*11, V1244fs*86, V4799M, A1390fs*42, A2169T, A2205fs*59, A221fs*40, A3552fs*4, C5123*, C5142fs*5, D2769N, E2962fs*42, F1790fs*12, F2494fs*49, F2566fs*17, G1317*, G1960fs*87, G1995*, G2262fs*37, G2265fs*21, G3189*, G3698fs*51, H77fs*53, I4491fs*1, I977fs*23, K1686fs*36, K304fs*30, K3140fs*2, K4843fs*15, L1020fs*36, L1271fs*15, L2331fs*46, L2594fs*97, L3716fs*296, L3880fs*131, L5183fs*16, L5318fs*14, P1460fs*46, P2206fs*58, P2382fs*2, P367fs*35, P4380fs*4, P444fs*2, P4968fs*27, P506fs*424, P583fs*347, P62fs*9, P886fs*44, Q1377R, Q1557*, Q211*, Q3471*, Q3811fs*201, Q3839fs*42, Q3909fs*103, Q3934*, Q3950R, Q4235fs*98, Q4284*, Q791fs*139, Q809fs*121, Q809fs*3, R1252*, R1687fs*4, R2099*, R2471*, R2771*, R2830*, R4198*, R466C, R5048H, R5086*, R5120C, R5282*, R5454*, R755fs*3, S102fs*28, S1107fs*12, S1684fs*38, S1684T, S2532fs*11, S2910fs*32, S3159fs*16, S4010fs*12, S4507fs*12, S456*, S4789fs*27, splice site 14644−1G>T, splice site 16413−2A>G, T209fs*11, T2191fs*11, T698fs*232, V1670fs*52, V3089fs*30, Y2199fs*65, or Y2907fs*3. In other embodiments, the alteration in a MSH3 gene comprises one or more of: K383fs*32, L564fs*1, E342*, K902fs*5, K99fs*3, N1020fs*17, N385fs*19, N524fs*3, N739fs*8, N861fs*6, or R268*. In other embodiments, the alteration in a PTCH1 gene comprises one or more of: S1203fs*52, R1308fs*64, Y1316fs*56, L39fs*41, R1308fs*17, E61fs*18, N97fs*43, V1164I, A563V, C1398fs*54, C727fs*19, E1242K, G526*, L50fs*39, N97fs*20, P643fs*11, R602*, or R6fs*1. In other embodiments, the alteration in a CDK12 gene comprises one or more of: G1461fs*38, Q1291fs*3, T1463fs*50, G1271fs*23, R983*, E59fs*33, E751*, E887*, H1035fs*7, I873fs*11, K445*, L342fs*8, L996*, N474fs*8, N864fs*2, P683fs*70, P686fs*13, P974L, Q115*, Q1418*, R1048*, R1331*, R298*, R890H, S133fs*24, splice site 1047−2A>C, T1346fs*7, or T212fs*18. In other embodiments, the alteration in a ARID1A gene comprises one or more of: D1850fs*33, D1850fs*4, F2141fs*59, G276fs*87, P1326fs*155, P224fs*8, Q766fs*67, K1072fs*21, Q1452fs*29, Q372fs*19, Q758fs*75, Q802fs*15, R1989*, A339fs*24, M1634fs*14, Q372fs*28, R693*, Y551fs*72, G314fs*49, P1115fs*46, P1568fs*44, P1898fs*25, Q1200*, Q1519fs*8, Q1631*, R1501fs*4, S1000Y, S11fs*91, W1073fs*32, A134fs*98, A1539fs*27, A27fs*24, A339fs*61, A62fs*39, A77fs*24, D1850fs*34, D2178fs*47, D2260fs*5, E1297*, E1733*, E1783fs*6, E2058*, E2120*, E992*, G1110fs*51, G122fs*278, G126fs*274, G1740*, G2069fs*50, G2087R, G236fs*163, G277fs*86, G285fs*78, G37fs*14, G801fs*32, G82fs*19, G83fs*28, G987fs*50, K1094fs*67, K1905fs*18, K250*, L1049fs*55, L1841fs*2, L2082fs*53, L2238fs*30, L2270fs*8, M1154fs*7, M1273fs*10, M1318fs*163, M1388fs*94, M1564fs*8, M1595fs*19, M1634fs*1, M890fs*46, N1313fs*168, N2109fs*26, P1175fs*5, P1451fs*41, P1468fs*13, P146fs*86, P1560fs*5, P225fs*175, P469fs*150, Q1188*, Q1212*, Q1250*, Q1327*, Q1327fs*11, Q1420*, Q1512*, Q1584*, Q1650*, Q1708*, Q1835*, Q1835fs*1, Q1974*, Q2115*, Q2176*, Q2176fs*48, Q288*, Q505fs*117, Q521*, Q538*, Q546fs*73, Q566*, Q575fs*46, Q581*, Q611*, Q633*, Q806fs*11, R1223C, R1335*, R1446*, R1461*, R1658fs*40, R1658W, R1722*, R1869fs*30, S1465fs*25, S1645fs*46, S2264*, S255fs*145, S366fs*25, S536fs*87, S617fs*2, S617fs*6, splice site 1921−3_1925delTAGGATCT, splice site 2879−2A>G, splice site 3715+1G>C, splice site 4005−2A>T, T1514fs*13, T1743M, T2252fs*27, T286fs*114, T894fs*25, V1561fs*11, V63fs*38, W1545*, W1670*, W2050*, W2091*, Y1101fs*1, Y1377*, Y2076*, Y222*, Y422*, or Y551fs*68. In other embodiments, the alteration in a ASXL1 gene comprises one or more of: G645fs*58, G646fs*12, R693*, E635fs*15, G646fs*58, Q592*, A627fs*8, E41K, E518*, E566*, E676*, E917*, F354L, G1376fs*74, G643fs*15, G643fs*61, G967del, L983fs*8, N1158fs*6, P1377fs*3, P763fs*12, P808fs*10, Q561fs*1, Q588*, Q695*, Q768fs*6, R541fs*162, R596fs*107, R718fs*7, S1335fs*115, S747fs*25, S892fs*16, splice site 140+2T>G, splice site 471+1G>A, T957fs*26, V737fs*10, W1037*, or W583*. In other embodiments, the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H. In other embodiments, the alteration in a BCORL1 gene comprises one or more of: P1681fs*20, A1166fs*56, G1682fs*4, A74fs*42, M644fs*4, A858fs*67, Q1001fs*49, R1299*, S803fs*83, A74fs*20, A971fs*4, E1655*, E619*, I389fs*29, K1207N, K1330fs*17, L275fs*143, N1412fs*38, P323fs*95, Q459*, R1196*, R1297*, R1338*, R1420*, R609*, R743fs*13, splice site 4306−2A>G, or W1105*. In other embodiments, the alteration in a CTNNB1 gene comprises one or more of: S45F, T41A, S45P, Q773*, R587*, S33C, T41I, D32N, E334K, G34E, N387K, R449C, S45A, T257L, W25*, W383R, D17_Q78del, D17_T75del, E568*, G69*, I35_G38del, I35S, K19_S37>N, K335fs*10, K335L, M8_V79del, R376H, R515Q, R582Q, R582W, R90*, R95*, S33F, S33T, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242-39del684, splice site 14−338_81>TTAC, splice site 14−39_225del251, splice site 14−48_241+12del288, splice site 14−5_97del89, splice site 14−6_241+74del308, splice site 14−69_242-10del488, splice site 14−7_89del83, splice site 14−80_241+22>CAT, splice site 14−91_241+25del344, splice site 1954+1G>A, splice site 1954+1G>T, splice site 1955−1G>A, splice site 2138−2A>C, splice site 241+1G>C, splice site 32_241+94del304, splice site 60_287del428, splice site 65_274del410, splice site 74_241+52del220, splice site 78_241+11del175, splice site 87_242−79del277, splice site 98_241+8>A, V22_S33del, V22_S37del, W25_I35>C, W383C, or W776*. In other embodiments, the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G. In other embodiments, the alteration in a CIC gene comprises one or more of: P1597fs*23, P1248fs*54, P509fs*14, P1116fs*45, T1375fs*40, P135fs*70, P1598fs*16, S1117fs*34, S961fs*6, A785fs*139, A900fs*24, D449fs*23, E367*, G136fs*8, G1600fs*14, P1128fs*33, P1336fs*3, P1529fs*91, P404fs*31, P515fs*8, P518fs*5, P574fs*154, P768fs*156, P911fs*13, P98fs*107, Q378*, R1313W, R201W, R353*, S902fs*21, S904fs*27, splice site 583−1G>A, T1541fs*18, T1541fs*79, or T328fs*78. In other embodiments, the alteration in a MAP3K1 gene comprises one or more of: C635*, E126*, E1293fs*3, E788*, G1074fs*8, G608fs*48, H114fs*50, K1160fs*12, L380fs*4, L915*, N1212fs*33, P74fs*3, Q1022*, Q320*, R208*, R288*, R307fs*5, R532*, S101fs*63, splice site 3983−1G>A, splice site 483−1G>A, splice site 633+2T>A, T1145fs*6, T457fs*31, V1045fs*12, or V569I. In other embodiments, the alteration in a ATR gene comprises one or more of: I774fs*5, R1814fs*10, F1091fs*28, F1134fs*6, I774fs*3, E148*, E1699*, E2579*, F2168*, F222fs*11, I1264fs*14, I691fs*5, I774fs*6, K446fs*11, K773fs*3, L1029fs*20, Q195*, R1015Q, R1814fs*8, R2001*, R223fs*1, R224fs*18, R2533*, R2547*, R2598*, S2207fs*15, S825fs*13, splice site 5381−1G>A, W1591*, or Y1844*. In other embodiments, the alteration in a MSH2 gene comprises one or more of: A230fs*16, C778fs*34, E188*, E86fs*4, R680*, S233fs*13, or Y408*. In other embodiments, the alteration in a CTCF gene comprises one or more of: T204fs*26, T204fs*18, R166C, E363fs*5, A137fs*17, A225V, D194fs*28, E112*, E145*, E182fs*9, G32fs*30, H19fs*15, MIT, N259fs*44, P50L, R11W, R275C, splice site 374−1G>T, splice site 854−1G>T, T317fs*91, Y15H, or Y195*. In other embodiments, the alteration in a JAK1 gene comprises one or more of: K860fs*16, P430fs*2, G741D, or K496N. In other embodiments, the alteration in a QKI gene comprises one or more of: K134fs*14, A313V, E42*, G77fs*14, L236fs*54, R319*, splice site 1010−233_*194del444, splice site 142+1G>A, splice site 143−1G>T, or splice site 546+1G>T. In other embodiments, the alteration in a CDH1 gene comprises one or more of: P126fs*89, P127fs*41, R492fs*44, S70fs*13, A241fs*3, A634V, C28*, D257G, D291N, D400G, G169fs*46, L214P, L711V, P372fs*8, Q16*, R335*, R63*, R74*, S111fs*6, S18fs*39, splice site 1137G>A, splice site 1138−1G>A, splice site 1565+1G>A, splice site 1711+2T>C, splice site 688−1G>T, T323fs*33, T340M, or W532*. In other embodiments, the alteration in a CASP8 gene comprises one or more of: R449*, K490fs*73, F373fs*26, I350fs*4, E212*, R452*, A197fs*14, P411L, R194fs*17, R68*, Y252*, A264fs*24, C196Y, D380fs*19, D380fs*2, E195*, E36*, F152fs*18, F296fs*11, GI IR, K478fs*10, K478fs*19, L59fs*12, L62P, N475fs*13, R250W, splice site 1355+2T>C, splice site 151+1G>A, V222fs*13, V268fs*8, V492fs*71, or Y8fs*1. In other embodiments, the alteration in a NOTCH3 gene comprises one or more of: A1802fs*8, C1344fs*76, P695fs*165, C43fs*32, G1318fs*245, G2035fs*50, P1317fs*103, T250fs*122, A1020fs*252, A1927T, C720fs*1, C87fs*149, D352fs*2, E1492fs*84, G2035fs*60, G2081fs*4, G707D, K2069fs*16, L2092fs*57, P2033fs*62, P2115fs*10, P42fs*194, R1589Q, R2031fs*54, R6fs*28, S1448fs*115, S157fs*5, splice site 119-156_197+47del282, splice site 1606+1G>A, splice site 5668−1G>T, T1098fs*174, or W1425*. In other embodiments, the alteration in a EP300 gene comprises one or more of: H2324fs*55, M1470fs*26, R1187H, splice site 1282+1G>A, C1385F, D1399N, H2324fs*29, L2303fs*74, L415P, R86*, C1408*, C1738*, E643fs*2, G54*, K1469fs*3, M1339fs*26, N1236fs*41, N419fs*12, Q2282*, Q498*, Q501fs*6, R1055*, R1281*, R1312*, R1627W, R2185*, R2263*, R2330fs*49, R580Q, R648*, S1214Y, S19fs*19, S2271fs*8, splice site 1169−2A>G, splice site 2379+1G>C, splice site 3671+1G>A, splice site 4453−2A>G, T1021fs*3, or Y1467H. In other embodiments, the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*. In other embodiments, the alteration in a MEN1 gene comprises one or more of: R521fs*43, E184V, L105fs*13, R457Q, R521fs*15, R532*, or splice site 799−9G>A. In other embodiments, the alteration in a BCOR gene comprises one or more of: P1587fs*53, Q1174fs*8, K1173fs*31, G400fs*42, R810*, Q1272fs*20, R1480*, S158fs*28, S336fs*45, C1329fs*45, D328fs*50, E1025*, E1030fs*48, E1182fs*6, E485fs*42, G154*, G906fs*5, G95fs*16, H1179fs*1, I1290fs*2, K1061fs*52, K1137fs*4, K1271fs*64, K1330*, K839fs*17, L279fs*21, N1425S, N390fs*53, N529fs*28, P602fs*67, P931fs*15, Q1174*, Q1274*, Q348*, Q430*, Q600*, R1053fs*26, R1181fs*1, R1498fs*36, R976*, S1371L, S336fs*42, splice site 166−2A>C, splice site 2997+1G>T, splice site 3239−2A>G, splice site 4072−2A>G, T1331fs*4, or V806fs*10.

In some embodiments, a Stage I, Stage II, or Stage III CRC is microsatellite stable (MSS) and/or does not comprise a POLE and/or POLD1 alteration. In some cases, a Stage I, Stage II, or Stage III CRC that is MSS and/or does not comprise a POLE and/or POLD1 alteration comprises an alteration in a CTNNB1 and/or MAP3K1 gene. In some embodiments, the alteration in a CTNNB1 gene comprises one or more of: T41A, S45F, Q773*, R587*, S33C, D32N, N387K, S45P, T257L, W25*, D17_Q78del, D17_T75del, I35_G38del, I35S, K19_S37>N, K335L, M8_V79del, R376H, R582Q, R582W, R90*, R95*, S33F, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242-39del684, splice site 14−338_81>TTAC, splice site 14−39_225del251, splice site 14−48_241+12del288, splice site 14−6_241+74del308, splice site 14−69_242-10del488, splice site 14−7_89del83, splice site 14−80_241+22>CAT, splice site 14−91_241+25del344, splice site 1954+1G>A, splice site 1954+1G>T, splice site 1955−1G>A, splice site 241+1G>C, splice site 32_241+94del304, splice site 60_287del428, splice site 65_274del410, splice site 74_241+52del220, splice site 78_241+11del175, splice site 87_242-79del277, splice site 98_241+8>A, T41L, V22_S33del, V22_S37del, W25_I35>C, W383C, W383R, or W776*. In some embodiments, the alteration in a MAP3K1 gene comprises one or more of: E126*, E1293fs*3, E788*, H114fs*50, L380fs*4, P74fs*3, Q1022*, R532*, S101fs*63, splice site 483−1G>A, T1145fs*6, or T457fs*31.

In some embodiments, the CRC is microsatellite stable (MSS) and/or does not have an alteration in a POLE and/or POLD1 gene, and the one or more biomarkers comprise an alteration in one or more of a BRAF, PTEN, or RNF43 gene. In some embodiments, the alteration in BRAF is a V600E, D594G, N581S, G466V, G469A, K483E, L485F, or L485S alteration, or any combination thereof. In some embodiments, the alteration in PTEN is a R233*, C136Y, D24fs*20, E242fs*9, E299*, H93R, K330*, L247*, L57fs*6, N323fs*2, N340fs*3, N63fs*11, P95L, Q171*, Q219*, Q245*, R130*, R130Q, splice site 209+5G>A, splice site 210−1G>C, splice site 626_634+2delGAACTTGCAGT, T131N, or V133I alteration, or any combination thereof. In some embodiments, the alteration in RNF43 is a Y332*, A193fs*6, A273fs*147, E258fs*162, M1I, P660fs*87, Q233*, Q426fs*77, Q8*, R117fs*41, R145*, R337*, splice site 375+1G>A, V271fs*11, W159*, W302*, Y248*, or Y332fs*110 alteration, or any combination thereof.

Any of the CRCs of the disclosure may comprise one or more biomarkers of the disclosure, such as one or more, or all, of (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof. In some embodiments, a CRC comprises one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, or SPEN, or any combination thereof. In some embodiments, a CRC comprises one or more, or all, of: (a) an MSI-H status, (b) a high TMB, and (c) a PD-L1 positive status. In some embodiments, any of the biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) may be detected or identified in any of the CRCs of the disclosure.

In some embodiments of any of the methods provided herein, the methods comprise acquiring knowledge of, identifying the presence of, or detecting in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof).

In some embodiments, acquiring knowledge of, detecting, or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence): identifies, classifies or predicts the individual as being at risk for CRC recurrence; identifies, classifies or predicts the individual as being at greater risk for CRC recurrence, for example, as compared to an individual with a CRC that does not comprise the one or more biomarkers; identifies, classifies or predicts the individual as being at risk for CRC recurrence and as likely to benefit from a treatment comprising an anti-cancer therapy; identifies, classifies or predicts the individual as being at risk for CRC recurrence and as likely to benefit from a chemotherapy; identifies, classifies or predicts the individual as being at risk for CRC recurrence, and (i) as a candidate to receive a treatment comprising an anti-cancer therapy, or (ii) as likely to respond to a treatment that comprises an anti-cancer therapy; identifies, classifies or predicts the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; identifies, classifies or predicts the individual as being at risk for early CRC recurrence; identifies, classifies or predicts the individual as being at greater risk for early CRC recurrence, as compared to an individual with a CRC that does not comprise the one or more biomarkers; identifies, classifies or predicts the individual as being at risk for CRC recurrence within about one year or less from the time of initial diagnosis of the CRC; and/or identifies, classifies or predicts the individual as being at greater risk for CRC recurrence within about one year or less from the time of initial diagnosis of the CRC, as compared to an individual with a CRC that does not comprise the one or more biomarkers.

In some embodiments, the methods of the disclosure comprise detecting, in one or more samples obtained from an individual at a first time point, the presence or absence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof). In some embodiments, the methods further comprise detecting, in one or more samples obtained from the individual at a second time point after the first time point, the presence or absence of one or more biomarkers of the disclosure. In some embodiments, the methods further comprise providing an assessment of CRC recurrence or CRC recurrence risk, in the individual based, at least in part, on the presence or absence of the one or more biomarkers in one or more samples at the first time point and/or at the second time point. In some embodiments, the presence of the one or more biomarkers in one or more samples at the first and/or second time point identifies the individual as having increased risk of CRC recurrence. In some embodiments, the methods further comprise selecting a treatment, administering a treatment, adjusting a treatment, adjusting a dose of a treatment, or applying a treatment to the individual based, at least in part, on detecting the presence of the one or more biomarkers in one or more samples at the first time point and/or at the second time point, wherein the treatment comprises an anti-cancer therapy, such as an anti-cancer therapy provided herein.

In some embodiments, the methods of the disclosure comprise performing DNA sequencing on one or more samples obtained from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) to determine a sequencing mutation profile. In some embodiments, the sequencing mutation profile identifies the presence or absence of one or more biomarkers (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in the one or more samples. In some embodiments, the sequencing mutation profile further includes sequence information on a group of genes, such as the presence or absence of an alteration in a gene in the group of genes. In some embodiments, the group of genes comprises one or more known/suspected oncogenes and/or tumor suppressors, one or more cancer-related genes, or any combination thereof. In some embodiments, the group of genes comprises at least 1, at least 2, at least 3, at least 4, at least 5, at least 6, at least 7, at least 8, at least 9, at least 10, at least 20, at least 30, at least 40, or more than 40 genes. Alternatively or additionally, in some embodiments, the group of genes comprises one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703, or any combination thereof. Alternatively or additionally, in some embodiments, the group of genes comprises one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB, or any combination thereof. In some embodiments, identifying the presence of the one or more biomarkers in the one or more samples identifies the individual as being at risk for CRC recurrence. In some embodiments, the methods further comprise identifying a candidate treatment for the individual, based at least in part on the sequencing mutation profile. In some embodiments, the candidate treatment comprises an anti-cancer therapy, such as an anti-cancer therapy provided herein. In some embodiments, the presence of the one or more biomarkers in the one or more samples identifies the individual as one who may benefit from a treatment comprising an anti-cancer therapy, e.g., an anti-cancer therapy provided herein. In some embodiments, the presence of the one or more biomarkers in the one or more samples predicts the individual to have longer survival when treated with a treatment comprising an anti-cancer therapy, e.g., as compared to an individual with a CRC that does not comprise the one or more biomarkers. In some embodiments, the DNA sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, such as next generation sequencing (NGS).

In some embodiments of any of the methods provided herein, the methods further comprise detecting or acquiring knowledge of the presence or absence of a cancer, such as a CRC, in a sample from the individual. In some embodiments, the methods comprise detecting or acquiring knowledge of the presence or absence of a cancer in a sample from the individual; and detecting or acquiring knowledge of the presence or absence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from the individual.

In some embodiments of any of the methods provided herein, responsive to acquiring knowledge of, detecting or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual, the methods comprise administering to the individual an effective amount of a treatment that comprises an anti-cancer therapy, such as an anti-cancer therapy provided herein.

In some embodiments of any of the methods provided herein, the methods further comprise generating a report comprising one or more treatment options identified for the individual based at least in part on detection of the one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual. In some embodiments, the one or more treatment options comprise an anti-cancer therapy, such as an anti-cancer therapy provided herein. In some embodiments, the report indicates the presence or absence of the one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual. In some embodiments, the report indicates, based at least in part on acquiring knowledge of, detecting, or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof), that the individual is at risk for CRC recurrence; that the individual is at greater risk for CRC recurrence, for example, as compared to an individual with a CRC that does not comprise the one or more biomarkers; that the individual is at risk for CRC recurrence and is likely to benefit from a treatment comprising an anti-cancer therapy; that the individual is at risk for CRC recurrence and is likely to benefit from a chemotherapy; that the individual is at risk for CRC recurrence, and (i) is a candidate to receive a treatment comprising an anti-cancer therapy, or (ii) is likely to respond to a treatment that comprises an anti-cancer therapy; that the individual is at risk for CRC recurrence and likely to have longer survival when treated with a treatment comprising an anti-cancer therapy, for example, as compared to survival of an individual with a CRC that does not comprise the one or more biomarkers and/or that is not treated with a treatment comprising said anti-cancer therapy; that the individual is at risk for early CRC recurrence; that the individual is at greater risk for early CRC recurrence, as compared to an individual with a CRC that does not comprise the one or more biomarkers; that the individual is at risk for CRC recurrence within about one year or less from the time of initial diagnosis of the CRC; and/or that the individual is at greater risk for CRC recurrence within about one year or less from the time of initial diagnosis of the CRC, as compared to an individual with a CRC that does not comprise the one or more biomarkers.

In some embodiments, acquiring knowledge of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) comprises detecting the one or more biomarkers in the one or more samples.

In some embodiments, the methods of the disclosure further comprise providing an assessment of the one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence).

In some embodiments of any of the methods provided herein, the methods further comprise acquiring knowledge, detecting or identifying the presence in a sample from the individual a base substitution, a short insertion/deletion (indel), a copy number alteration, or a genomic rearrangement in one or more genes, such as one or more known/suspected oncogenes and/or tumor suppressors, one or more cancer-related genes, or any combination thereof. In some embodiments, the one or more genes comprise at least 1, at least 2, at least 3, at least 4, at least 5, at least 6, at least 7, at least 8, at least 9, at least 10, at least 20, at least 30, at least 40, or more than 40 genes. Alternatively or additionally, in some embodiments, the one or more genes comprise one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703, or any combination thereof. Alternatively or additionally, in some embodiments, the one or more gene comprise one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB, or any combination thereof.

In some embodiments of any of the methods provided herein, acquiring knowledge, detecting or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) identifies the individual as one who should be monitored or assessed for CRC recurrence more frequently, as compared to an individual with a CRC that does not comprise the one or more biomarkers. In some embodiments of any of the methods provided herein, acquiring knowledge, detecting or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) identifies the individual as a candidate to receive a more aggressive anti-cancer therapy for CRC and/or an anti-cancer therapy for CRC of greater duration, as compared to an individual with a CRC that does not comprise the one or more biomarkers. In some embodiments of any of the methods provided herein, acquiring knowledge, detecting or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) identifies the individual as one who: (a) should be administered a standard-of-care treatment for CRC, optionally a more aggressive standard-of-care treatment for CRC and/or a standard-of-care treatment for CRC of greater duration, as compared to an individual with a CRC that does not comprise the one or more biomarkers; or (b) should be administered an anti-cancer therapy other than a standard-of-care treatment for CRC, or an anti-cancer therapy combined with a standard-of-care treatment for CRC. In some embodiments, the standard-of-care treatment for CRC comprises a chemotherapy combined with an anti-VEGF agent or an anti-EGFR agent. In some embodiments, the chemotherapy comprises a leucovorin calcium (folinic acid), fluorouracil, and oxaliplatin combination (FOLFOX). In some embodiments, the anti-VEGF agent is an anti-VEGF antibody, such as bevacizumab. In some embodiments, the anti-EGFR agent is an anti-EGFR antibody, such as cetuximab.

In some embodiments, the anti-cancer therapy according to the present disclosure comprises a chemotherapy (e.g., 5-fluorouracil (5-FU), irinotecan, oxaliplatin, capecitabine, or Trifluridine/Tipiracil, or any combination thereof), a radiation therapy, an immunotherapy, a targeted therapy, a surgery, or any combination thereof. In some embodiments, the anti-cancer therapy comprises a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), a tyrosine kinase inhibitor therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA). In some specific embodiments, the anti-cancer therapy comprises a chemotherapeutic agent.

In some embodiments, the one or more biomarkers comprise an MSI-H status, a high TMB, and/or a PD-L1 positive status, and the anti-cancer therapy comprises an immunotherapy. In some embodiments, acquiring knowledge, detecting or identifying the presence of an MSI-H status, a high TMB, and/or a PD-L1 positive status in one or more samples from the individual indicates that the individual is likely to benefit from a treatment comprising an immunotherapy; that the individual is a candidate to receive a treatment comprising an immunotherapy, or is likely to respond to a treatment that comprises an immunotherapy; and/or that the individual is likely to have longer survival when treated with a treatment comprising an immunotherapy, for example, as compared to survival of an individual with a CRC that does not comprise an MSI-H status, a high TMB, and/or a PD-L1 positive status and/or that is not treated with a treatment comprising said immunotherapy.

In some embodiments, the one or more biomarkers comprise an alteration in a BRAF gene, and the anti-cancer therapy comprises a BRAF-targeted therapy, an EGFR-targeted therapy, or a combination thereof. In some embodiments, acquiring knowledge, detecting or identifying the presence of an alteration in a BRAF gene in one or more samples from the individual indicates that the individual is likely to benefit from a treatment comprising a BRAF-targeted therapy, an EGFR-targeted therapy, or a combination thereof, that the individual is a candidate to receive a treatment comprising a BRAF-targeted therapy, an EGFR-targeted therapy, or a combination thereof, or is likely to respond to a treatment that comprises a BRAF-targeted therapy, an EGFR-targeted therapy, or a combination thereof; and/or that the individual is likely to have longer survival when treated with a treatment comprising a BRAF-targeted therapy, an EGFR-targeted therapy, or a combination thereof, for example, as compared to survival of an individual with a CRC that does not comprise an alteration in a BRAF gene and/or that is not treated with a treatment comprising a BRAF-targeted therapy, an EGFR-targeted therapy, or a combination thereof. In some embodiments, the BRAF-targeted therapy comprises an anti-EGFR agent or a combination of a chemotherapy and an anti-EGFR agent. In some embodiments, the anti-EGFR agent is an anti-EGFR antibody.

In some embodiments, the one or more biomarkers comprise an alteration in a PTEN gene, and the anti-cancer therapy comprises a PTEN-targeted therapy. In some embodiments, acquiring knowledge, detecting or identifying the presence of an alteration in a PTEN gene in one or more samples from the individual indicates that the individual is likely to benefit from a treatment comprising a PTEN-targeted therapy; that the individual is a candidate to receive a treatment comprising a PTEN-targeted therapy, or is likely to respond to a treatment that comprises a PTEN-targeted therapy; and/or that the individual is likely to have longer survival when treated with a treatment comprising a PTEN-targeted therapy, for example, as compared to survival of an individual with a CRC that does not comprise an alteration in a PTEN gene and/or that is not treated with a treatment comprising a PTEN-targeted therapy.

In some embodiments, the one or more biomarkers comprise an alteration in a RNF43 gene, and the anti-cancer therapy comprises a RNF43-targeted therapy, a BRAF-targeted therapy, an EGFR-targeted therapy, a MEK-targeted therapy, or a combination thereof. In some embodiments, acquiring knowledge, detecting or identifying the presence of an alteration in a RNF43 gene in one or more samples from the individual indicates that the individual is likely to benefit from a treatment comprising a RNF43-targeted therapy, a BRAF-targeted therapy, an EGFR-targeted therapy, a MEK-targeted therapy, or a combination thereof, that the individual is a candidate to receive a treatment comprising a RNF43-targeted therapy, a BRAF-targeted therapy, an EGFR-targeted therapy, a MEK-targeted therapy, or a combination thereof, or is likely to respond to a treatment that comprises a RNF43-targeted therapy, a BRAF-targeted therapy, an EGFR-targeted therapy, a MEK-targeted therapy, or a combination thereof, and/or that the individual is likely to have longer survival when treated with a treatment comprising a RNF43-targeted therapy, a BRAF-targeted therapy, an EGFR-targeted therapy, a MEK-targeted therapy, or a combination thereof, for example, as compared to survival of an individual with a CRC that does not comprise an alteration in an RNF43 gene and/or that is not treated with a treatment comprising a RNF43-targeted therapy, a BRAF-targeted therapy, an EGFR-targeted therapy, a MEK-targeted therapy, or a combination thereof.

In some embodiments, the one or more biomarkers comprise an alteration in a ASXL1 gene, and the anti-cancer therapy comprises a ASXL1-targeted therapy. In some embodiments, acquiring knowledge, detecting or identifying the presence of an alteration in a ASXL1 gene in one or more samples from the individual indicates that the individual is likely to benefit from a treatment comprising a ASXL1-targeted therapy; that the individual is a candidate to receive a treatment comprising a ASXL1-targeted therapy, or is likely to respond to a treatment that comprises a ASXL1-targeted therapy; and/or that the individual is likely to have longer survival when treated with a treatment comprising a ASXL1-targeted therapy, for example, as compared to survival of an individual with a CRC that does not comprise an alteration in a ASXL1 gene and/or that is not treated with a treatment comprising a ASXL1-targeted therapy.

In some embodiments, the one or more biomarkers comprise an alteration in a CREBBP gene, and the anti-cancer therapy comprises a CREBBP-targeted therapy. In some embodiments, acquiring knowledge, detecting or identifying the presence of an alteration in a CREBBP gene in one or more samples from the individual indicates that the individual is likely to benefit from a treatment comprising a CREBBP-targeted therapy; that the individual is a candidate to receive a treatment comprising a CREBBP-targeted therapy, or is likely to respond to a treatment that comprises a CREBBP-targeted therapy; and/or that the individual is likely to have longer survival when treated with a treatment comprising a CREBBP-targeted therapy, for example, as compared to survival of an individual with a CRC that does not comprise an alteration in a CREBBP gene and/or that is not treated with a treatment comprising a CREBBP-targeted therapy.

In some embodiments, the one or more biomarkers comprise an alteration in a MLL2 gene, and the anti-cancer therapy comprises a MLL2-targeted therapy. In some embodiments, acquiring knowledge, detecting or identifying the presence of an alteration in a MLL2 gene in one or more samples from the individual indicates that the individual is likely to benefit from a treatment comprising a MLL2-targeted therapy; that the individual is a candidate to receive a treatment comprising a MLL2-targeted therapy, or is likely to respond to a treatment that comprises a MLL2-targeted therapy; and/or that the individual is likely to have longer survival when treated with a treatment comprising a MLL2-targeted therapy, for example, as compared to survival of an individual with a CRC that does not comprise an alteration in a MLL2 gene and/or that is not treated with a treatment comprising a MLL2-targeted therapy.

In some embodiments, the one or more biomarkers comprise an alteration in a BCORL1 gene, and the anti-cancer therapy comprises a BCORL1-targeted therapy. In some embodiments, acquiring knowledge, detecting or identifying the presence of an alteration in a BCORL1 gene in one or more samples from the individual indicates that the individual is likely to benefit from a treatment comprising a BCORL1-targeted therapy; that the individual is a candidate to receive a treatment comprising a BCORL1-targeted therapy, or is likely to respond to a treatment that comprises a BCORL1-targeted therapy; and/or that the individual is likely to have longer survival when treated with a treatment comprising a BCORL1-targeted therapy, for example, as compared to survival of an individual with a CRC that does not comprise an alteration in a BCORL1 gene and/or that is not treated with a treatment comprising a BCORL1-targeted therapy.

In some embodiments, the one or more biomarkers comprise an alteration in an ATR gene, and the anti-cancer therapy comprises an ATR-targeted therapy. In some embodiments, acquiring knowledge, detecting or identifying the presence of an alteration in an ATR gene in one or more samples from the individual indicates that the individual is likely to benefit from a treatment comprising an ATR-targeted therapy; that the individual is a candidate to receive a treatment comprising an ATR-targeted therapy, or is likely to respond to a treatment that comprises an ATR-targeted therapy; and/or that the individual is likely to have longer survival when treated with a treatment comprising an ATR-targeted therapy, for example, as compared to survival of an individual with a CRC that does not comprise an alteration in an ATR gene and/or that is not treated with a treatment comprising an ATR-targeted therapy.

In some embodiments, the one or more biomarkers comprise an alteration in a SPEN gene, and the anti-cancer therapy comprises a SPEN-targeted therapy. In some embodiments, acquiring knowledge, detecting or identifying the presence of an alteration in a SPEN gene in one or more samples from the individual indicates that the individual is likely to benefit from a treatment comprising a SPEN-targeted therapy; that the individual is a candidate to receive a treatment comprising a SPEN-targeted therapy, or is likely to respond to a treatment that comprises a SPEN-targeted therapy; and/or that the individual is likely to have longer survival when treated with a treatment comprising a SPEN-targeted therapy, for example, as compared to survival of an individual with a CRC that does not comprise an alteration in a SPEN gene and/or that is not treated with a treatment comprising a SPEN-targeted therapy.

In some embodiments, the one or more biomarkers comprise an alteration in a BRCA1 gene, and the anti-cancer therapy comprises a BRCA1-targeted therapy. In some embodiments, acquiring knowledge, detecting or identifying the presence of an alteration in a BRCA1 gene in one or more samples from the individual indicates that the individual is likely to benefit from a treatment comprising a BRCA1-targeted therapy; that the individual is a candidate to receive a treatment comprising a BRCA1-targeted therapy, or is likely to respond to a treatment that comprises a BRCA1-targeted therapy; and/or that the individual is likely to have longer survival when treated with a treatment comprising a BRCA1-targeted therapy, for example, as compared to survival of an individual with a CRC that does not comprise an alteration in a BRCA1 gene and/or that is not treated with a treatment comprising a BRCA1-targeted therapy.

In some embodiments, the one or more biomarkers comprise an alteration in a BRCA2 gene, and the anti-cancer therapy comprises a BRCA2-targeted therapy. In some embodiments, acquiring knowledge, detecting or identifying the presence of an alteration in a BRCA2 gene in one or more samples from the individual indicates that the individual is likely to benefit from a treatment comprising a BRCA2-targeted therapy; that the individual is a candidate to receive a treatment comprising a BRCA2-targeted therapy, or is likely to respond to a treatment that comprises a BRCA2-targeted therapy; and/or that the individual is likely to have longer survival when treated with a treatment comprising a BRCA2-targeted therapy, for example, as compared to survival of an individual with a CRC that does not comprise an alteration in a BRCA2 gene and/or that is not treated with a treatment comprising a BRCA2-targeted therapy.

In some embodiments, the one or more biomarkers comprise an alteration in a MSH6 gene, and the anti-cancer therapy comprises a MSH6-targeted therapy. In some embodiments, acquiring knowledge, detecting or identifying the presence of an alteration in a MSH6 gene in one or more samples from the individual indicates the individual is likely to benefit from a treatment comprising a MSH6-targeted therapy; that the individual is a candidate to receive a treatment comprising a MSH6-targeted therapy, or is likely to respond to a treatment that comprises a MSH6-targeted therapy; and/or that the individual is likely to have longer survival when treated with a treatment comprising a MSH6-targeted therapy, for example, as compared to survival of an individual with a CRC that does not comprise an alteration in a MSH6 gene and/or that is not treated with a treatment comprising a MSH6-targeted therapy.

In some embodiments, an anti-cancer therapy of the disclosure is a first-line anti-cancer therapy for the CRC.

In some embodiments of any of the methods provided herein, the treatment or the one or more treatment options, e.g., the anti-cancer therapy, further comprises an additional anti-cancer therapy. In some embodiments of any of the methods provided herein, the treatment or the one or more treatment options further comprise administering an additional anti-cancer therapy to the individual. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, the individual has been previously treated, or is being treated, for cancer, e.g., for a CRC, with a treatment for cancer, e.g., an anti-cancer therapy described herein or any other anti-cancer therapy or treatment known in the art. In some embodiments, the cancer, e.g., the CRC, progressed on a prior treatment. In some embodiments, the cancer, e.g., the CRC, is refractory to a prior anti-cancer therapy. In some embodiments, the individual has not been previously treated for cancer, e.g., for a CRC.

In some embodiments of any of the methods provided herein, the methods further comprise generating a molecular profile for the individual or the sample, based, at least in part, on detecting the presence or absence of the one or more biomarkers described herein (e.g., an MSI-H status, a high TMB, a PD-L1 positive status, and/or an alteration in one or more genes of the disclosure). In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments of any of the methods provided herein, the methods further comprise selecting a treatment, administering a treatment, or applying a treatment to the individual based on the generated molecular profile, wherein the treatment comprises an anti-cancer therapy, e.g., as described herein. In some embodiments of any of the methods provided herein, the methods further comprise generating a report indicating the presence or absence of the one or more biomarkers described herein (e.g., an MSI-H status, a high TMB, a PD-L1 positive status, and/or an alteration in one or more genes of the disclosure) in the sample. In some embodiments of any of the methods provided herein, the methods further comprise generating, by one or more processors, a report indicating the presence or absence of the one or more biomarkers described herein (e.g., an MSI-H status, a high TMB, a PD-L1 positive status, and/or an alteration in one or more genes of the disclosure) in the sample. In some embodiments, the report comprises the generated molecular profile. In some embodiments, the methods further comprise providing or transmitting the report, e.g., as described below. In some embodiments, the report is transmitted via a computer network or a peer-to-peer connection. In some instances, all or a portion of the report may be displayed in a graphical user interface of an online or web-based healthcare portal.

In some embodiments of any of the methods provided herein, the methods for determining the presence or absence of the one or more biomarkers described herein (e.g., an MSI-H status, a high TMB, a PD-L1 positive status, and/or an alteration in one or more genes of the disclosure), may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., CRC. In some instances, the variant panel selected for genomic profiling may comprise the detection of variant sequences at a selected set of gene loci.

In some instances, the variant panel selected for genomic profiling may comprise detection of variant sequences at a number of gene loci (e.g., one or more genes) through comprehensive genomic profiling (CGP), a next-generation sequencing (NGS) approach used to assess hundreds of genes (including relevant cancer biomarkers) in a single assay. Inclusion of the disclosed methods for determining the presence or absence of the one or more biomarkers described herein (e.g., an MSI-H status, a high TMB, a PD-L1 positive status, and/or an alteration in one or more genes of the disclosure) as part of a genomic profiling process can improve the validity of, e.g., disease detection calls by, for example, independently confirming the presence of the one or more biomarkers described herein (e.g., an MSI-H status, a high TMB, a PD-L1 positive status, and/or an alteration in one or more genes of the disclosure) in a given patient sample.

Also provided herein are reagents for detecting one or more biomarkers of the disclosure in one or more samples from an individual.

In some embodiments, provided herein are bait molecules suitable for the detection of one or more biomarkers of the disclosure, for example, an alteration in one or more genes of the disclosure, an MSI-H status, a high TMB, and/or a PD-L1 positive status. In some embodiments, a bait molecule comprises a capture nucleic acid molecule configured to hybridize to a target nucleic acid molecule. In some embodiments, the capture nucleic acid molecule is configured to hybridize to a nucleotide sequence corresponding to the one or more genes of the disclosure, e.g., BRAF, PTEN, RNF43, ASXL1, CREBBP, MLL2, BCORL1, ATR, SPEN, BRCA1, BRCA2, or MSH6. In some embodiments, the capture nucleic acid molecule is configured to hybridize to a fragment of one or more genes of the disclosure, e.g., BRAF, PTEN, RNF43, ASXL1, CREBBP, MLL2, BCORL1, ATR, SPEN, BRCA1, BRCA2, or MSH6. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a biomarker of the disclosure, such as an alteration in one or more genes of the disclosure. In some embodiments, the fragment comprises any of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more, nucleotides in length. In some embodiments, the fragment comprises between about 5 and about 100 nucleotides, between about 10 and about 50 nucleotides, or between about 10 and about 20 nucleotides, including any specific value within each of the recited ranges.

In some embodiments, the capture nucleic acid molecule comprises (or is) between about 5 and about 25 nucleotides, between about 10 and about 30 nucleotides, between about 5 and about 300 nucleotides, between about 100 and about 300 nucleotides, between about 130 and about 230 nucleotides, between about 100 and about 500 nucleotides, between about 100 and about 200 nucleotides, between about 50 and about 1000 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is configured to hybridize to a biomarker of the disclosure, such as an alteration in one or more genes of the disclosure, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the biomarker, e.g., the alteration. In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification. The baits described herein can be used for selection of exons and short target sequences.

In some embodiments, provided herein are probes, e.g., nucleic acid molecules, suitable for the detection of one or more biomarkers of the disclosure, for example, an alteration in one or more genes of the disclosure, an MSI-H status, a high TMB, and/or a PD-L1 positive status. In some embodiments, a probe provided herein comprises a nucleic acid sequence configured to hybridize to a target nucleic acid molecule. In some embodiments, the probe comprises a nucleic acid sequence configured to hybridize to a nucleotide sequence corresponding to the one or more genes of the disclosure, e.g., BRAF, PTEN, RNF43, ASXL1, CREBBP, MLL2, BCORL1, ATR, SPEN, BRCA1, BRCA2, or MSH6. In some embodiments, the probe comprises a nucleic acid sequence configured to hybridize to a fragment or portion of one or more genes of the disclosure, e.g., BRAF, PTEN, RNF43, ASXL1, CREBBP, MLL2, BCORL1, ATR, SPEN, BRCA1, BRCA2, or MSH6. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a biomarker of the disclosure, such as an alteration in one or more genes of the disclosure, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the biomarker, e.g., the alteration. In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization. In some embodiments, probes provided herein may be used according to the methods of detection of a biomarker of the disclosure described herein. For example, a probe provided herein may be used for detecting an alteration in one or more genes of the disclosure, e.g., in a sample obtained from an individual. In some embodiments, the probe may be used for identifying cells or tissues that express a biomarker of the disclosure. In some embodiments, a probe provided herein specifically hybridizes to a nucleic acid molecule comprising a rearrangement (e.g., a deletion, inversion, insertion, duplication, or other rearrangement) resulting in an alteration in a gene of the disclosure. In some embodiments, a probe of the disclosure distinguishes a nucleic acid, e.g., a genomic or transcribed nucleic acid, e.g., a cDNA or RNA, having a biomarker of the disclosure, from a reference nucleotide sequence, e.g., a nucleotide sequence not having the biomarker, e.g., an alteration in a gene of the disclosure.

Also provided herein are isolated pairs of allele-specific probes, wherein, for example, the first probe of the pair specifically hybridizes to a nucleic acid molecule comprising a biomarker of the disclosure, such as an alteration in a gene of the disclosure, and the second probe of the pair specifically hybridizes to a corresponding wild type sequence. Probe pairs can be designed and produced for any of the biomarkers described herein and are useful in detecting a somatic mutation in a sample. In some embodiments, a first probe of a pair specifically hybridizes to an alteration (e.g., the breakpoint of an alteration, rearrangement, inversion, duplication, deletion, insertion or translocation resulting in an alteration in a gene of the disclosure), and a second probe of a pair specifically hybridizes to a sequence upstream or downstream of the mutation. In some embodiments, one or more probes provided herein are suitable for use in in situ hybridization methods, e.g., as described above, such as FISH. Chromosomal probes, e.g., for use in the FISH methods described herein, are typically about 50 to about 105 nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a biomarker of the disclosure, e.g., an alteration in a gene of the disclosure. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)-or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as 32P and 3H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, provided herein are oligonucleotides, e.g., useful as primers, suitable for the detection of one or more biomarkers of the disclosure, for example, an alteration in one or more genes of the disclosure, an MSI-H status, a high TMB, and/or a PD-L1 positive status. In some embodiments, an oligonucleotide, e.g., a primer, provided herein comprises a nucleotide sequence configured to hybridize to a target nucleic acid molecule. In some embodiments, the oligonucleotide comprises a nucleotide sequence configured to hybridize to a nucleotide sequence corresponding to the one or more genes of the disclosure, e.g., BRAF, PTEN, RNF43, ASXL1, CREBBP, MLL2, BCORL1, ATR, SPEN, BRCA1, BRCA2, or MSH6. In some embodiments, the oligonucleotide comprises a nucleotide sequence configured to hybridize to a fragment or portion of one or more genes of the disclosure, e.g., BRAF, PTEN, RNF43, ASXL1, CREBBP, MLL2, BCORL1, ATR, SPEN, BRCA1, BRCA2, or MSH6. In some embodiments, the oligonucleotide, e.g., the primer, comprises a nucleotide sequence configured to hybridize to a biomarker of the disclosure, e.g., an alteration in a gene of the disclosure, and may be further configured to hybridize to between about 10 and about 12, about 12 and about 15, about 15 and about 17, about 17 and about 20, about 20 and about 25, or about 25 and about 30, or more nucleotides flanking either side of the biomarker, e.g., the alteration. In some embodiments, the oligonucleotide comprises a nucleotide sequence corresponding to a gene of the disclosure, or a fragment thereof. In some embodiments, the fragment comprises between about 10 and about 30 nucleotides, between about 12 and about 20 nucleotides, or between about 12 and about 17 nucleotides. In some embodiments, the oligonucleotide comprises a nucleotide sequence complementary to a gene of the disclosure, or a fragment thereof. In some embodiments, the fragment comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur. In some embodiments, an oligonucleotide, e.g., a primer, provided herein may be useful for initiating DNA synthesis via PCR (polymerase chain reaction) or a sequencing method. In some embodiments, pairs of oligonucleotides, e.g., pairs of primers, are provided herein, which are configured to hybridize to a nucleic acid molecule that comprises a biomarker of the disclosure, such as an alteration in a gene of the disclosure. In some embodiments, a pair of oligonucleotides of the disclosure may be used for directing amplification, e.g., using a PCR reaction. In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, comprising a biomarker of the disclosure, e.g., an alteration in a gene of the disclosure, from a reference nucleotide sequence, e.g., a nucleotide sequence not having the biomarker, e.g., the alteration. In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in a biomarker of the disclosure, such as an alteration in a gene of the disclosure. In another aspect, provided herein is a primer or primer set for amplifying a nucleic acid molecule comprising an alteration, rearrangement, chromosomal inversion, insertion, deletion, translocation, duplication or other rearrangement resulting in a biomarker of the disclosure, e.g., an alteration in a gene of the disclosure. In certain aspects, provided herein are allele-specific oligonucleotides, e.g., primers, wherein a first oligonucleotide of a pair specifically hybridizes to an alteration (e.g., an alteration in a gene of the disclosure), and a second oligonucleotide of a pair specifically hybridizes to a sequence upstream or downstream of the alteration. In certain aspects, provided herein are pairs of oligonucleotides, e.g., primers, wherein a first oligonucleotide of a pair specifically hybridizes to a sequence upstream of an alteration (e.g., an alteration in a gene of the disclosure), and a second oligonucleotide of the pair specifically hybridizes to a sequence downstream of the alteration.

In some embodiments, provided herein are antibodies or antibody fragments suitable for the detection of one or more biomarkers of the disclosure, for example, an alteration in one or more genes of the disclosure, an MSI-H status, a high TMB, and/or a PD-L1 positive status. In some embodiments, the antibodies or antibody fragments may be used for detecting a PD-L1 positive status, e.g., by detecting levels of PD-L1 protein using an anti-PD-L1 antibody or antibody fragment, e.g., using immunohistochemistry as described herein or known in the art. In some embodiments, the antibodies may be used for detecting an MSI-H status, for example, using anti-MLH1, anti-MSH2, anti-MSH6, and/or anti-PMS2 antibodies or antibody fragments, e.g., using immunohistochemistry. Antibodies may also be used for detecting a polypeptide or protein encoded by a gene of the disclosure (e.g., BRAF, PTEN, RNF43, ASXL1, CREBBP, MLL2, BCORL1, ATR, SPEN, BRCA1, BRCA2, or MSH6) comprising an alteration as described herein. The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits the desired antigen binding activity.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., 125I, 131I, 35S or 3H.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen. In some embodiments, a RIA is performed with the Fab version of an antibody of the disclosure and its antigen. In certain embodiments, an antibody provided herein is an antibody fragment.

Antibody fragments include, but are not limited to, Fab, Fab′, Fab′-SH, F(ab′)2, Fv, and single-chain antibody molecule (e.g., scFv) fragments, and other fragments described herein or known in the art. In certain embodiments, an antibody provided herein is a diabody, a triabody or a tetrabody. In certain embodiments, an antibody provided herein is a single-domain antibody. Antibody fragments can be made by various techniques, including but not limited to proteolytic digestion of an intact antibody, as well as production by recombinant host cells (e.g., E. coli or phage), as known in the art and as described herein. In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof. In certain embodiments, a chimeric antibody is a humanized antibody. Humanized antibodies and methods of making them are known in the art. In certain embodiments, an antibody provided herein is a human antibody. Human antibodies can be produced using various techniques known in the art. Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain embodiments, an antibody provided herein is a multispecific antibody, e.g., a bispecific antibody. Multispecific antibodies are monoclonal antibodies that have binding specificities for at least two different sites or at least two different antigens. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fcs; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF.”

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding. In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC). In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function. In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

The disclosed methods may be used with any of a variety of samples, e.g., as described in further detail herein. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof. In some embodiments, the methods further comprise obtaining the sample, e.g., from the individual or from the cancer, e.g., the CRC.

In some instances, nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample. In some embodiments of any of the methods provided herein, the method further comprises determining the circulating tumor DNA (ctDNA) fraction of a liquid biopsy sample.

### C. Samples

A variety of materials can be the source of, or serve as, samples for use in any of the methods of the disclosure.

For example, the sample can be, or be derived from: solid tissue, such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a cancer, such as a CRC described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is or comprises a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids or proteins extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may be or be derived from a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample. In some embodiments of any of the methods provided herein, the method further comprises determining the circulating tumor DNA (ctDNA) fraction of a liquid biopsy sample.

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a CRC, suspected of having a CRC, being tested for CRC, being treated for CRC, having CRC recurrence risk, suspected of having CRC recurrence risk, being tested for CRC recurrence risk, or being treated for CRC recurrence. In some embodiments, the methods provided herein further comprise obtaining one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence). In some embodiments, one or more of the samples from the individual are obtained about 3 months or less, about 2 months or less, or about 1 month or less from the time of diagnosis of a CRC in the individual. In some embodiments, one or more of the samples from the individual are obtained about 12 weeks or less, about 11 weeks or less, about 10 weeks or less, about 9 weeks or less, about 8 weeks or less, about 7 weeks or less, about 6 weeks or less, about 5 weeks or less, about 4 weeks or less, about 3 weeks or less, about 2 weeks or less, or about 1 week or less from the time of diagnosis of a CRC in the individual. In some embodiments, the one or more samples are obtained or derived from a CRC in the individual. In some embodiments, the one or more samples are obtained prior to the individual having received a treatment for the CRC. In other embodiments, the one or more samples are obtained after the individual has received a treatment for the CRC.

In some embodiments, the sample is a normal control sample or a reference sample, such as from a non-tumor or cancer sample, e.g., a normal control such as a blood control, a normal adjacent tumor (NAT), or any other non-cancerous sample from the same or a different individual.

### D. Anti-Cancer Therapies

Certain aspects of the present disclosure relate to anti-cancer therapies, as well as use of biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) for: identifying an individual at risk for CRC recurrence who may benefit from a treatment comprising an anti-cancer therapy; selecting a therapy or treatment for an individual at risk for CRC recurrence; identifying one or more treatment options for an individual at risk for CRC recurrence; predicting survival (e.g., length of survival) of an individual having a CRC treated with a treatment comprising an anti-cancer therapy; treating or delaying progression of a CRC; identifying, classifying or predicting an individual as being at risk for CRC recurrence and as likely to benefit from a chemotherapy; identifying, classifying or predicting an individual as being at risk for CRC recurrence, and (i) as a candidate to receive a treatment comprising an anti-cancer therapy, or (ii) as likely to respond to a treatment that comprises an anti-cancer therapy; identifying, classifying or predicting an individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; identifying, classifying or predicting the individual as being at risk for early CRC recurrence; identifying, classifying or predicting the individual as being at greater risk for early CRC recurrence, as compared to an individual with a CRC that does not comprise the one or more biomarkers; identifying, classifying or predicting the individual as being at risk for CRC recurrence within about one year or less from the time of initial diagnosis of the CRC; and/or identifying, classifying or predicting the individual as being at greater risk for CRC recurrence within about one year or less from the time of initial diagnosis of the CRC, as compared to an individual with a CRC that does not comprise the one or more biomarkers. The present disclosure also provides uses for anti-cancer therapies (e.g., in methods of treating or delaying progression of a CRC in an individual, or in methods for manufacturing a medicament for treating or delaying progression of a CRC).

In some instances, the methods of the disclosure can include administering an anti-cancer therapy or applying an anti-cancer therapy to an individual based, at least in part, on detecting, acquiring knowledge of, or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence). In other instances, the methods of the disclosure can include administering an anti-cancer therapy or applying an anti-cancer therapy to an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) based on a generated molecular and/or sequencing mutation profile. An anti-cancer therapy can refer to a compound that is effective in the treatment of cancer cells. Examples of anti-cancer agents or anti-cancer therapies include, but are not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMNR) pathway.

In some embodiments, an anti-cancer therapy of the disclosure is a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), a tyrosine kinase inhibitor therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA). In some specific embodiments, the anti-cancer therapy comprises a chemotherapeutic agent.

In some embodiments, the anti-cancer therapy is an immunotherapy, also known as a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21, 2013). In some embodiments, such cancer vaccines augment the anti-cancer response.

In some embodiments, the cancer vaccine comprises a polynucleotide that encodes a neoantigen, e.g., a neoantigen expressed by the cancer to be targeted. In some embodiments, the cancer vaccine comprises DNA that encodes a neoantigen, e.g., a neoantigen expressed by the cancer to be targeted. In some embodiments, the cancer vaccine comprises RNA that encodes a neoantigen, e.g., a neoantigen expressed by the cancer to be targeted. In some embodiments, the cancer vaccine comprises a polynucleotide that encodes a neoantigen, e.g., a neoantigen expressed by the cancer to be targeted. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g., human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen expressed by a cancer of the disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen expressed by a cancer of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen expressed by a cancer of the disclosure, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen expressed by a cancer of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises an oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g., via intratumoral, intraperitoneal, intravenous, intra-arterial, intramuscular, intradermal, intracranial, subcutaneous, or intranasal administration.

In some embodiments, the immunotherapy is a checkpoint inhibitor, i.e., an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular, chimerized, humanized or human forms of antibodies may be used. In some embodiments, the immune checkpoint inhibitor is monovalent and/or monospecific. In some embodiments, the immune checkpoint inhibitor is multivalent and/or multispecific.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the checkpoint inhibitor is a PD-1 binding antagonist, such as a molecule that inhibits the binding of PD-1 to its ligand binding partners. In a specific embodiment, the PD-1 ligand binding partners are PD-L1 and/or PD-L2. In another instance, the checkpoint inhibitor is a PD-L1 binding antagonist, such as a molecule that inhibits the binding of PD-L1 to its binding ligands. In a specific embodiment, PD-L1 binding partners are PD-1 and/or B7-1. In another instance, the checkpoint inhibitor is a PD-L2 binding antagonist, such as a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1 128Z-E27, REMD-288, SG-001, BY-24.3, CB-201, IBI-319, ONCR-177, Max-1, CS-4100, JBI-426, CCC-0701, or CCX-4503, or derivatives thereof. In some specific embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof. In some specific embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises a chemotherapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-ll); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein transferase inhibitors, transplatinum, and pharmaceutically acceptable salts, acids, or derivatives of any of the above. In some embodiments, the chemotherapy comprises a leucovorin calcium (folinic acid), fluorouracil, and oxaliplatin combination (FOLFOX). In some embodiments, the chemotherapy comprises 5-fluorouracil (5-FU), irinotecan, oxaliplatin, capecitabine, or Trifluridine/Tipiracil, or any combination thereof.

In some embodiments, the chemotherapy comprises an antimetabolite chemotherapeutic agent. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the chemotherapy comprises a platinum-based chemotherapeutic agent. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy of the disclosure is a BRAF-targeted therapy. In some embodiments, the BRAF-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for BRAF-positive or BRAF-rearranged cancer, a BRAF-targeted therapy being tested in a clinical trial, a treatment for BRAF-positive or BRAF-rearranged cancer being tested in a clinical trial, a MAPK pathway inhibitor, or any combination thereof. In some embodiments, the BRAF-targeted therapy is a kinase inhibitor known in the art or described herein. In some embodiments, the BRAF-targeted therapy is a tyrosine kinase inhibitor known in the art or described herein. In some embodiments, the BRAF-targeted therapy is a serine/threonine kinase inhibitor known in the art or described herein. In some embodiments, the BRAF-targeted therapy is a multi-kinase inhibitor or a BRAF-specific inhibitor known in the art or described herein. In some embodiments, the kinase inhibitor inhibits the kinase activity of a BRAF polypeptide. In some embodiments, the BRAF-targeted therapy is a class I, class II, class III and/or a pan-Raf BRAF inhibitor. In some embodiments, the BRAF-targeted therapy comprises one or more of sorafenib, PLX4720, PLX-3603, dabrafenib, encorafenib, GDC-0879, RAF265, XL281, ARQ736, BAY73-4506, vemurafenib, regorafenib, CEP-32496, EBI-907, AZ304, BGB-283, or BAY 43-9006. In some embodiments, the BRAF-targeted therapy comprises a MAPK pathway inhibitor, for example, an inhibitor of a receptor tyrosine kinase, RAS, MEK, and/or ERK. In some embodiments, the MEK inhibitor comprises one or more of trametinib, cobimetinib, binimetinib, selumetinib, or R05126766. In some embodiments, the ERK inhibitor comprises one or more of BVD-523, CC-90003, GDC-0994, KO-947, LY-3214996, or MK-8353. In some embodiments, the RAS inhibitor comprises one or more of AMG 510, MRTX849, ARS-3248, or LY3499446. In some embodiments, the BRAF-targeted therapy comprises an EGFR-targeted therapy.

In some embodiments, the BRAF-targeted therapy is sorafenib. In some embodiments, the BRAF-targeted therapy is PLX4720. In some embodiments, the BRAF-targeted therapy is PLX-3603. In some embodiments, the BRAF targeted therapy is GDC-0879. In some embodiments, the BRAF targeted therapy is RAF265. In some embodiments, the BRAF targeted therapy is XL281. In some embodiments, the BRAF targeted therapy is ARQ736. In some embodiments, the BRAF targeted therapy is BAY73-4506. In some embodiments, the BRAF targeted therapy is regorafenib. In some embodiments, the BRAF targeted therapy is CEP-32496. CEP-32496 is a multikinase binding compound, but exhibits selective cellular cytotoxicity for BRAF^{V600E} cells (James et al., CEP-32496: A Novel Orally Active BRAFV600E Inhibitor with Selective Cellular and In Vivo Antitumor Activity. Mol Cancer Ther 1 Apr. 2012; 11 (4): 930-941). In some embodiments, the BRAF targeted therapy is EBI-907. EBI-907 exhibits potent anti-tumor activity in vivo. (Zhang et al., EBI-907, a novel BRAF^{V600E} inhibitor, has potent oral anti-tumor activity and a broad kinase selectivity profile, Cancer Biology & Therapy 2016, 17:2, 199-207). In some embodiments, the BRAF targeted therapy is AZ304. AZ304 inhibits both BRAF^{wildtype} and BRAF^{V600E} (Ma, R., Xu, L., Qu, X. et al. AZ304, a novel dual BRAF inhibitor, exerts anti-tumour effects in colorectal cancer independently of BRAF genetic status. Br J Cancer 118, 1453-1463 (2018)). In some embodiments, the BRAF targeted therapy is BGB-283. BGB-283 exhibits anti-tumor activity in vivo. (Tang et al., BGB-283, a Novel RAF Kinase and EGFR Inhibitor, Displays Potent Antitumor Activity in BRAF-Mutated Colorectal Cancers. Mol Cancer Ther 1 Oct. 2015; 14 (10): 2187-2197). In some embodiments, the BRAF-targeted therapy is vemurafenib. Vemurafenib has demonstrated activity for patients with various tumor types harboring BRAF V600 mutations (Khaddour, Karam, et al. “Vemurafenib.” StatPearls, StatPearls Publishing, 29 Aug. 2022.). In some embodiments, the BRAF-targeted therapy is dabrafenib. In some embodiments, the BRAF-targeted therapy is encorafenib. In some embodiments, the BRAF-targeted therapy is PLX4032. In some embodiments, the BRAF-targeted therapy is BAY43-9006. In some embodiments, the BRAF-targeted therapy comprises one or more of belvarafenib, PF-07799933, encorafinib, PF-07284890, PLX7904, PLX8394, vemurafenib, dabrafenib, sorafenib, naporafenib, PLX4720, PLX-3603, GDC-0879, RAF265, XL281, ARQ736, BAY73-4506, regorafenib, CEP-32496, EBI-907, AZ304, BGB-283, or KIN-2787. In some embodiments, the BRAF-targeted therapy comprises one or more of sorafenib, PLX4720, PLX-3603, dabrafenib (GSK2118436), encorafenib (LGX818), GDC-0879, RAF265, XL281, ARQ736, BAY73-4506, vemurafenib (e.g., Zelboraff®), cobimetinib (e.g., Cotellic®), binimetinib (e.g., Mektovi®), regorafenib (e.g., Stivarga®), selumetinib (e.g., Koselugo®), trametinib (e.g., Mekinist®), or BAY 43-9006.

In some embodiments, the anti-cancer therapy of the disclosure is a PTEN-targeted therapy. In some embodiments, the PTEN-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a PTEN alteration, a PTEN-targeted therapy being tested in a clinical trial, a treatment for cancer with a PTEN alteration being tested in a clinical trial, or any combination thereof. In some embodiments, the PTEN-targeted therapy is a PI3K inhibitor, a HDAC inhibitor, a PLK inhibitor, an mTOR inhibitor, a TORC 1 inhibitor, a TORC 2 inhibitor, an AKT inhibitor, or a PARP inhibitor. In some embodiments, the PTEN-targeted therapy comprises one or more of BKM120, XL-147, PX-866, GDC-0941, BAY80-6946, CH5132799, GDC-0084, ZSTK474, GDC-0032, BYL719, MLN1117, GSK2636771, Rigosertib, CUDC-907, PKI-587 (PF-05212384), BEZ235, BGT226, GDC-0980, XL-765, SF1126, LY3023414, Everolimus (RAD001), Temsirolimus (CCI-779), Ridaforolimus (AP-23573), MLN0128, AZD-2014, CC-223, AZD-5363, LY2780301, GDC-0068, GSK2110183, MK-2206, Olaparib, Veliparib, Iniparib, Rucaparib, CEP-9722, E7016, or E7449.

In some embodiments, the anti-cancer therapy of the disclosure is an RNF43-targeted therapy. In some embodiments, the RNF43-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a RNF43 alteration, a RNF43-targeted therapy being tested in a clinical trial, a treatment for cancer with a RNF43 alteration being tested in a clinical trial, or any combination thereof. In some embodiments, the RNF43-targeted therapy is a Wnt inhibitor. In some embodiments, the RNF43-targeted therapy is a PORCN inhibitor, an anti-FZD antibody or an anti-R-spondin (RSPO) antibody. In some embodiments, the RNF43-targeted therapy is an anti-BRAF/EGFR therapy known in the art or described herein. In some embodiments, the RNF43-targeted therapy is a BRAF-targeted therapy, an EGFR-targeted therapy, a MEK-targeted therapy, or a combination thereof.

In some embodiments, the anti-cancer therapy of the disclosure is an ASXL1-targeted therapy. In some embodiments, the ASXL1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a ASXL1 alteration, a ASXL1-targeted therapy being tested in a clinical trial, a treatment for cancer with a ASXL1 alteration being tested in a clinical trial, or any combination thereof. In some embodiments, the ASXL1-targeted therapy is a BAP1 complex inhibitor. In some embodiments, the ASXL1-targeted therapy is a BET bromodomain inhibitor.

In some embodiments, the anti-cancer therapy of the disclosure is a CREBBP-targeted therapy. In some embodiments, the CREBBP-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a CREBBP alteration, a CREBBP-targeted therapy being tested in a clinical trial, a treatment for cancer with a CREBBP alteration being tested in a clinical trial, or any combination thereof.

In some embodiments, the anti-cancer therapy of the disclosure is an MLL2-targeted therapy. In some embodiments, the MLL2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a MLL2 alteration, a MLL2-targeted therapy being tested in a clinical trial, a treatment for cancer with a MLL2 alteration being tested in a clinical trial, or any combination thereof. In some embodiments, the MLL2-targeted therapy is a histone deacetylase (HDAC) inhibitor. In some embodiments, the HDAC inhibitor comprises orinostat or abexinostat. In some embodiments, the MLL2-targeted therapy is a glycolysis inhibitor known in the art or described herein. In some embodiments, the glycoslysis inhibitor comprises 2-deoxy-D-glucose, POMHEX (an enolasel inhibitor), or lonidamine (a hexokinase inhibitor).

In some embodiments, the anti-cancer therapy of the disclosure is a BCORL1-targeted therapy. In some embodiments, the BCORL1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a BCORL1 alteration, a BCORL1-targeted therapy being tested in a clinical trial, a treatment for cancer with a BCORL1 alteration being tested in a clinical trial, or any combination thereof.

In some embodiments, the anti-cancer therapy of the disclosure is an ATR-targeted therapy. In some embodiments, the ATR-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a ATR alteration, a ATR-targeted therapy being tested in a clinical trial, a treatment for cancer with a ATR alteration being tested in a clinical trial, or any combination thereof. In some embodiments, the ATR-targeted therapy comprises an anti-DNA repair therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026). In some embodiments, the ATR-targeted therapy is an DNA-PK inhibitor.

In some embodiments, the anti-cancer therapy of the disclosure is an SPEN-targeted therapy. In some embodiments, the SPEN-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a SPEN alteration, a SPEN-targeted therapy being tested in a clinical trial, a treatment for cancer with a SPEN alteration being tested in a clinical trial, or any combination thereof. In some embodiments, the SPEN-targeted therapy comprises tamoxifen.

In some embodiments, the anti-cancer therapy of the disclosure is a BRCA1-targeted therapy. In some embodiments, the BRCA1-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a BRCA1 alteration, a BRCA1-targeted therapy being tested in a clinical trial, a treatment for cancer with a BRCA1 alteration being tested in a clinical trial, or any combination thereof. In some embodiments, the BRCA1-targeted therapy is a PARP inhibitor. In some embodiments, the BRCA1-targeted therapy comprises one or more of Olaparib (Lynparza), talazoparib (Talzenna), niraparib, or rucaparib.

In some embodiments, the anti-cancer therapy of the disclosure is a BRCA2-targeted therapy. In some embodiments, the BRCA2-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a BRCA2 alteration, a BRCA2-targeted therapy being tested in a clinical trial, a treatment for cancer with a BRCA2 alteration being tested in a clinical trial, or any combination thereof. In some embodiments, the BRCA2-targeted therapy is a PARP inhibitor. In some embodiments, the BRCA2-targeted therapy comprises one or more of Olaparib (Lynparza), talazoparib (Talzenna), niraparib, or rucaparib.

In some embodiments, the anti-cancer therapy of the disclosure is an MSH6-targeted therapy. In some embodiments, the MSH6-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer with a MSH6 alteration, a MSH6-targeted therapy being tested in a clinical trial, a treatment for cancer with a MSH6 alteration being tested in a clinical trial, or any combination thereof. In some embodiments, the MSH6-targeted therapy is a histone decateylase/HDAC inhibitor. In some embodiments, the MSH6-targeted therapy comprises one or more of abexinostat, pembrolizumab, nivolumab, or nivolumab in combination with ipilimumab.

In some embodiments, an anti-cancer therapy of the disclosure is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Some non-limiting examples of chemotherapeutic agents which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, an anti-cancer therapy of the disclosure comprises a cyclin-dependent kinase (CDK) inhibitor. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, R05126766, WX-544, and HL-085, as well as pharmaceutically acceptable salts thereof. In some embodiments, the anti-cancer therapy inhibits one or more activities of the Raf/MEK/ERK pathway, including inhibitors of a receptor tyrosine kinase (e.g., EGFR or ERBB2), Raf, MEK, and/or ERK.

In some embodiments, an anti-cancer therapy of the disclosure comprises a mammalian target of rapamycin (mTOR) inhibitor. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), PX-866, as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, C1A, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, an anti-cancer therapy of the disclosure comprises an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, an anti-cancer therapy of the disclosure comprises one or more anti-inflammatory agents. In some embodiments, the anti-inflammatory agent is an interleukin and/or an interferon.

In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some embodiments, an anti-cancer therapy of the disclosure comprises a kinase inhibitor. Examples of kinase inhibitors include those that target one or more receptor tyrosine kinases, e.g., BCR-ABL, EGFR, HER-2/ErbB2, HER-3/ErbB3, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, PLX3397, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa). A non-limiting example of a HER-3 inhibitor is KTN3379.

In some embodiments, an anti-cancer therapy of the disclosure comprises any of abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, an anti-cancer therapy of the disclosure comprises an anti-angiogenic agent. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-7, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina ν β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, an anti-cancer therapy of the disclosure comprises an anti-DNA repair therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, an anti-cancer therapy of the disclosure comprises a radiosensitizer. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, an anti-cancer therapy of the disclosure comprises an anti-inflammatory agent. In some embodiments, the anti-inflammatory agent is an agent that blocks, inhibits, or reduces inflammation or signaling from an inflammatory signaling pathway. In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, an anti-cancer therapy of the disclosure comprises an anti-hormonal agent. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some aspects, provided herein are therapeutic formulations comprising an anti-cancer therapy provided herein and pharmaceutically acceptable carriers, excipients, or stabilizers. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

### E. Reporting

In some embodiments, the methods provided herein comprise generating a report, and/or providing a report to party.

In some embodiments, a report according to the present disclosure comprises information about one or more of: (1) one or more biomarkers of the disclosure, such as one or more of: (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof; (2) a CRC of the disclosure; (3) risk or likelihood of recurrence of a CRC in an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence); (4) a treatment, a therapy, or one or more treatment options for an individual, e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence.

In some embodiments, a report according to the present disclosure comprises information about the presence or absence of one or more biomarkers of the disclosure, such as one or more of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, in one or more samples obtained from an individual, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence. In one embodiment, a report according to the present disclosure indicates that one or more biomarkers of the disclosure is present in a sample obtained from the individual. In one embodiment, a report according to the present disclosure indicates that one or more biomarkers of the disclosure is not present in a sample obtained from the individual. In one embodiment, a report according to the present disclosure indicates that one or more biomarkers of the disclosure have been detected in a sample obtained from the individual. In one embodiment, a report according to the present disclosure indicates that one or more biomarkers of the disclosure has not been detected in a sample obtained from the individual. In some embodiments, the report comprises an identifier for the individual from which the sample was obtained.

In some embodiments, the report includes: information on the role of one or more biomarkers of the disclosure, such as one or more of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, in disease, such as in CRC, and/or in recurrence of disease, e.g., CRC recurrence. Such information can include one or more of: information on prognosis of a CRC in an individual; information on resistance of a CRC to one or more treatments; information on potential or suggested therapeutic options (e.g., such as an anti-cancer therapy provided herein, or a treatment selected or identified according to the methods provided herein); information on the risk or likelihood of CRC recurrence; or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as an additional anti-cancer therapy). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining one or more samples, such as a sample described herein, from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence); detecting or acquiring knowledge of the presence of one or more biomarkers of the disclosure in one or more samples from the individual, such as one or more of: (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, and generating a report. In some embodiments, a report generated according to the methods provided herein comprises one or more of: information about the presence or absence of one or more biomarkers of the disclosure in one or more samples, such as one or more of: (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, information on the risk or likelihood of CRC recurrence; an identifier for the individual from which the sample(s) was obtained; information on the role of the one or more biomarkers of the disclosure, or its wild type counterparts, in disease (e.g., such as in CRC and/or CRC recurrence); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., an additional anti-cancer therapy). In some embodiments, the report generated is a personalized cancer report.

A report according to the present disclosure may be in an electronic, web-based, or paper form. The report may be provided to an individual or a patient (e.g., an individual or patient having a CRC, suspected of having a CRC, being tested for CRC, being treated for CRC, having CRC recurrence risk, suspected of having CRC recurrence risk, being tested for CRC recurrence risk, or being treated for CRC recurrence), or to an individual or entity other than the individual or patient, such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from the individual. In some embodiments, the report is provided or delivered to an individual or entity within any of about 1 day or more, about 7 days or more, about 14 days or more, about 21 days or more, about 30 days or more, about 45 days or more, or about 60 days or more from detecting or acquiring knowledge of one or more biomarkers of the disclosure in one or more samples from the individual, such as one or more of: (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof.

### F. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

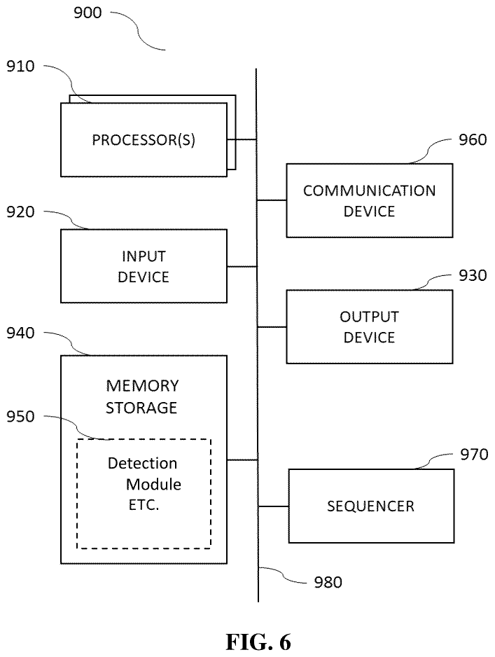

FIG. 6 illustrates an example of a computing device or system in accordance with one embodiment. Device 900 can be a host computer connected to a network. Device 900 can be a client computer or a server. As shown in FIG. 6, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting one or more biomarkers of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting one or more biomarkers of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

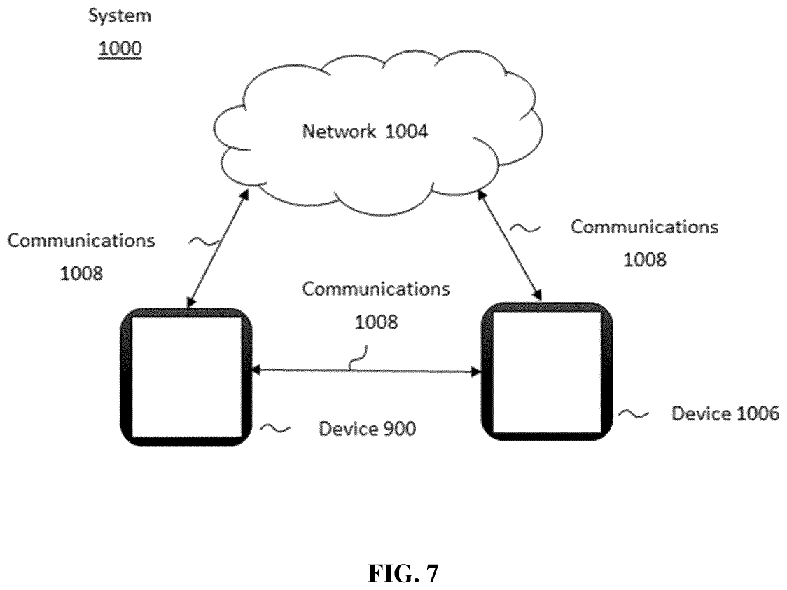

Device 900 may be connected to a network (e.g., network 1004, as shown in FIG. 7 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 7 illustrates an example of a computing system in accordance with one embodiment. In computing system 1000, device 900 (e.g., as described above and illustrated in FIG. 6) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

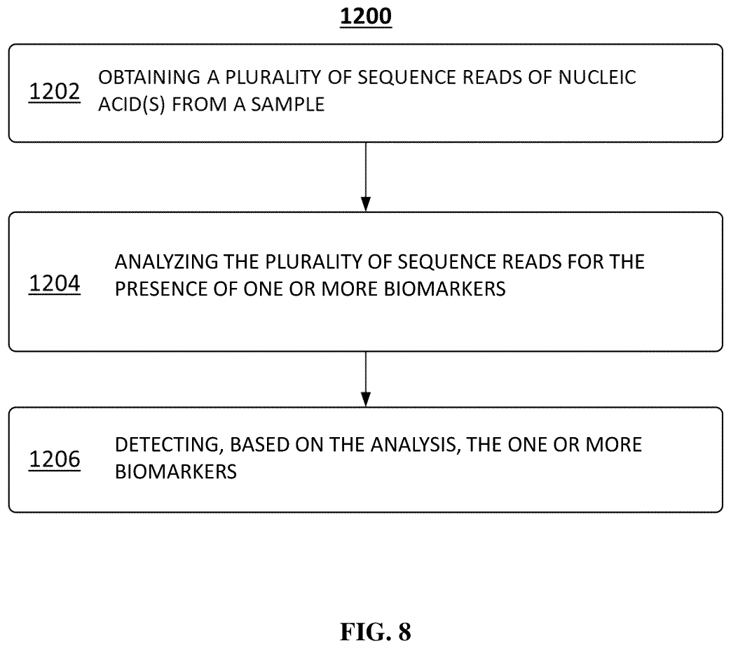

FIG. 8 illustrates an exemplary process 1200 for detecting one or more biomarkers of the disclosure (such as one or more of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from one or more samples obtained from an individual, e.g., as described herein, such as an individual having a CRC, suspected of having a CRC, being tested for CRC, being treated for CRC, having CRC recurrence risk, suspected of having CRC recurrence risk, being tested for CRC recurrence risk, or being treated for CRC recurrence. In some embodiments, the sequence reads are obtained using a sequencer, e.g., as described herein or otherwise known in the art. Optionally, prior to obtaining the sequence reads, the sample is purified, enriched, and/or subjected to PCR amplification. At block 1204, an exemplary system (e.g., one or more electronic devices) analyzes the plurality of sequence reads for the presence of one or more biomarkers of the disclosure (such as one or more of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof). At block 1206, the system detects (e.g., based on the analysis) one or more biomarkers of the disclosure in the one or more samples.

In some embodiments of any of the methods, systems, devices, non-transitory computer readable storage media, or processes of the disclosure, acquiring knowledge, detecting or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence) identifies the individual as one who should be monitored or assessed for CRC recurrence more frequently, as compared to an individual with a CRC that does not comprise the one or more biomarkers; identifies the individual as a candidate to receive a more aggressive anti-cancer therapy for CRC and/or an anti-cancer therapy for CRC of greater duration, as compared to an individual with a CRC that does not comprise the one or more biomarkers; and/or identifies the individual as one who: (a) should be administered a standard-of-care treatment for CRC, optionally a more aggressive standard-of-care treatment for CRC and/or a standard-of-care treatment for CRC of greater duration, as compared to an individual with a CRC that does not comprise the one or more biomarkers; or (b) should be administered an anti-cancer therapy other than a standard-of-care treatment for CRC, or an anti-cancer therapy combined with a standard-of-care treatment for CRC. In some embodiments, the standard-of-care treatment for CRC comprises a chemotherapy combined with an anti-VEGF agent or an anti-EGFR agent. In some embodiments, the chemotherapy comprises a leucovorin calcium (folinic acid), fluorouracil, and oxaliplatin combination (FOLFOX). In some embodiments, the anti-VEGF agent is an anti-VEGF antibody, such as bevacizumab. In some embodiments, the anti-EGFR agent is an anti-EGFR antibody, such as cetuximab. In some embodiments of any of the methods, systems, devices, non-transitory computer readable storage media, or processes of the disclosure, acquiring knowledge of, detecting, or identifying the presence of one or more biomarkers of the disclosure (e.g., one or more, or all, of: (a) a microsatellite instability high (MSI-H) status, (b) a high tumor mutational burden (TMB), (c) a PD-L1 positive status, and (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in one or more samples from an individual (e.g., an individual having a CRC, suspected of having a CRC, being tested for CRC, being treated for CRC, having CRC recurrence risk, suspected of having CRC recurrence risk, being tested for CRC recurrence risk, or being treated for CRC recurrence): identifies, classifies or predicts the individual as being at risk for CRC recurrence; identifies, classifies or predicts the individual as being at greater risk for CRC recurrence, for example, as compared to an individual with a CRC that does not comprise the one or more biomarkers; identifies, classifies or predicts the individual as being at risk for CRC recurrence and as likely to benefit from a treatment comprising an anti-cancer therapy; identifies, classifies or predicts the individual as being at risk for CRC recurrence and as likely to benefit from a chemotherapy; identifies, classifies or predicts the individual as being at risk for CRC recurrence, and (i) as a candidate to receive a treatment comprising an anti-cancer therapy, or (ii) as likely to respond to a treatment that comprises an anti-cancer therapy; identifies, classifies or predicts the individual to be at risk for CRC recurrence and to have longer survival when treated with a treatment comprising an anti-cancer therapy, for example, as compared to survival of an individual with a CRC that does not comprise the one or more biomarkers and/or that is not treated with a treatment comprising said anti-cancer therapy; identifies, classifies or predicts the individual as being at risk for early CRC recurrence; identifies, classifies or predicts the individual as being at greater risk for early CRC recurrence, as compared to an individual with a CRC that does not comprise the one or more biomarkers; identifies, classifies or predicts the individual as being at risk for CRC recurrence within about one year or less from the time of initial diagnosis of the CRC; and/or identifies, classifies or predicts the individual as being at greater risk for CRC recurrence within about one year or less from the time of initial diagnosis of the CRC, as compared to an individual with a CRC that does not comprise the one or more biomarkers.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, an individual is administered a treatment, e.g., an anti-cancer therapy, based at least in part on detection of one or more biomarkers of the disclosure in one or more samples from the individual.

In some embodiments of any of the methods, systems, devices, non-transitory computer readable storage media, or processes of the disclosure, the disclosed methods may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., a CRC. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods as part of a genomic profiling process can improve the validity of, e.g., disease detection calls, CRC recurrence risk assessments, etc., made on the basis of the genomic profiling by, for example, independently confirming the presence of the one or more biomarkers of the disclosure in a given patient sample. In some instances, the comprehensive genomic profiling may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the comprehensive genomic profiling may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof. In some embodiments of any of the methods, systems, devices, non-transitory computer readable storage media, or processes of the disclosure, a molecular profile for a sample or for an individual is generated based at least in part on detecting one or more biomarkers of the disclosure in one or more samples. In some instances, the molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the molecular profile may comprise results from a comprehensive genomic profiling (CGP) test (e.g., as describe above), a nucleic acid sequencing-based test, a gene expression profiling test, a cancer hotspot panel test, a DNA methylation test, a DNA fragmentation test, an RNA fragmentation test, or any combination thereof. In some embodiments, the molecular profile further comprises/indicates/comprises information on presence or absence of mutations in one or more additional genes, e.g., a panel of known/suspected oncogenes and/or tumor suppressors. In some embodiments, the one or more additional genes comprise at least 1, at least 2, at least 3, at least 4, at least 5, at least 6, at least 7, at least 8, at least 9, at least 10, at least 20, at least 30, at least 40, or more than 40 genes. Alternatively or additionally, in some embodiments, the one or more additional genes comprise one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703, or any combination thereof. Alternatively or additionally, in some embodiments, the one or more additional genes comprise one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1p, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB, or any combination thereof. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors, one or more cancer-related genes, or any combination thereof. In some embodiments, the one or more genes or gene loci comprise one or more known/suspected oncogenes and/or tumor suppressors, one or more cancer-related genes, or any combination thereof. In some embodiments, the individual is administered a treatment based at least in part on the molecular profile, such as an anti-cancer therapy as described herein.

In some embodiments of any of the methods, systems, devices, non-transitory computer readable storage media, or processes of the disclosure, a report is generated, e.g., as described in further detail herein. In some embodiments, the report comprises/indicates/comprises information on the presence or absence of one or more biomarkers of the disclosure in one or more samples from the individual. In some embodiments, the report comprises/indicates/comprises information on results of a genomic profiling process of a cancer, e.g., a CRC, in an individual (e.g., in one or more samples from the individual), e.g., as described above. In some embodiments, the report comprises/indicates/comprises information on results of comprehensive genomic profiling of a cancer, e.g., a CRC, in an individual (e.g., in one or more samples from the individual), e.g., as described above. In some embodiments, the report comprises/indicates/comprises information on a molecular profile generated for the individual or the sample, e.g., as described above. In some embodiments, the report comprises/indicates/comprises information on a treatment or one or more treatment options selected or identified for the individual, based, at least in part, on the presence of one or more biomarkers of the disclosure in one or more samples from the individual, and optionally based on results of a genomic profiling process, comprehensive genomic profiling, and/or a molecular profile generated for the individual or a sample, e.g., as described above. In some embodiments, the treatment or one or more treatment options comprise an anti-cancer therapy as described herein. In some embodiments, the report is provided or transmitted to the individual, a caregiver, a healthcare provider, a physician, an oncologist, an electronic medical record system, a hospital, a clinic, a third-party payer, an insurance company, or a government office, e.g., as described in further detail above. In some embodiments, the report is generated by one or more processors of a device, e.g., as described herein. In some embodiments, the report is transmitted via a computer network or a peer-to-peer connection. In some embodiments, an individual is administered a treatment based, at least in part, on the report. In some instances, all or a portion of the report may be displayed in a graphical user interface of an online or web-based healthcare portal.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. ARTICLES OF MANUFACTURE AND KITS

Further provided herein are kits or articles of manufacture comprising an anti-cancer therapy, such as an anti-cancer therapy described herein, and a package insert comprising instructions for using the anti-cancer therapy in a method of treating or delaying progression of cancer such as a CRC, e.g., by administration to an individual from whom one or more samples comprising one or more biomarkers of the disclosure (e.g., one or more, or all, of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof) has been obtained. In some embodiments, the anti-cancer therapy is any of the anti-cancer therapies described herein for use in any of the methods for treating or delaying progression of cancer of the disclosure.

Also provided herein are kits or articles of manufacture comprising one or more reagents for detecting one or more biomarkers of the disclosure (e.g., one or more of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof) in a sample. The kits or articles of manufacture may comprise one or more oligonucleotides, primers, probes, baits, antibodies or antibody fragments for detecting one or more of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, e.g., according to any detection method known in the art or described herein. In some embodiments, the kit is for use according to any method of detecting a biomarker of the disclosure known in the art or described herein, such as immunohistochemistry, sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting the one or more biomarkers of the disclosure in one or more samples, e.g., using the one or more oligonucleotides, primers, probes, baits, antibodies or antibody fragments.

The kit or article of manufacture may include, for example, a container and a label or package insert on or associated with the container. Suitable containers include, for example, bottles, vials, syringes, and the like. The container may be formed from a variety of materials such as glass or plastic. The container may hold or contain (1) a composition comprising one or more reagents for detecting one or more biomarkers of the disclosure, such as one or more oligonucleotides, primers, probes, baits, antibodies or antibody fragments for detecting one or more of (a) a MSI-H status, (b) a high TMB, (c) a PD-L1 positive status, and/or (d) an alteration in one or more of a BRAF, PTEN, RNF43, ASXL1, CREBBP, MLL2, BCORL1, ATR, SPEN, BRCA1, BRCA2, or MSH6 gene, or any combination thereof, and/or (2) one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure may also include information or instructions, for example in the form of a package insert or label, indicating that the one or more reagents and/or anti-cancer therapies are used for detecting one or more biomarkers of the disclosure, and/or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. EXEMPLARY EMBODIMENTS

The following exemplary embodiments are representative of some aspects of the invention:

Exemplary Embodiment 1: A method of identifying an individual at risk for colorectal cancer (CRC) recurrence who may benefit from a treatment comprising an anti-cancer therapy, the method comprising detecting in one or more samples from the individual one or more biomarkers selected from:
(a) a microsatellite instability high (MSI-H) status,
(b) a high tumor mutational burden (TMB),
(c) a PD-L1 positive status, and
(d) an alteration in one or more genes, wherein the one or more genes comprise BRAF, PTEN, RNF43, ASXL1, CREBBP, MLL2, BCORL1, ATR, or SPEN, or any combination thereof,
wherein detection of the one or more biomarkers in the one or more samples identifies the individual as being at risk for CRC recurrence and as likely to benefit from a treatment comprising an anti-cancer therapy.

Exemplary Embodiment 2: A method of identifying an individual at risk for CRC recurrence who may benefit from a treatment comprising an anti-cancer therapy, the method comprising detecting in one or more samples from the individual one or more biomarkers selected from:
(a) an MSI-H status,
(b) a high TMB,
(c) a PD-L1 positive status, and
(d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof;
wherein detection of the one or more biomarkers in the one or more samples identifies the individual as being at risk for CRC recurrence and as likely to benefit from a treatment comprising an anti-cancer therapy.

Exemplary Embodiment 3: A method of identifying an individual at risk for CRC recurrence who may benefit from a treatment comprising an anti-cancer therapy, the method comprising detecting in one or more samples from the individual one or more biomarkers selected from:
(a) an MSI-H status,
(b) a high TMB, and
(c) a PD-L1 positive status;
wherein detection of the one or more biomarkers in the one or more samples identifies the individual as being at risk for CRC recurrence and as likely to benefit from a treatment comprising an anti-cancer therapy.

Exemplary Embodiment 4: A method of selecting a therapy for an individual at risk for CRC recurrence, the method comprising detecting in one or more samples from the individual one or more biomarkers selected from:
(a) an MSI-H status,
(b) a high TMB,
(c) a PD-L1 positive status, and
(d) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as one who is at risk for CRC recurrence and may benefit from a treatment comprising an anti-cancer therapy.

Exemplary Embodiment 5: A method of selecting a therapy for an individual at risk for CRC recurrence, the method comprising detecting in one or more samples from the individual one or more biomarkers selected from:
(a) an MSI-H status,
(b) a high TMB,
(c) a PD-L1 positive status, and
(d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as one who is at risk for CRC recurrence and may benefit from a treatment comprising an anti-cancer therapy.

Exemplary Embodiment 6: A method of selecting a therapy for an individual at risk for CRC recurrence, the method comprising detecting in one or more samples from the individual one or more biomarkers selected from:
(a) an MSI-H status,
(b) a high TMB, and
(c) a PD-L1 positive status; and
wherein detection of the one or more biomarkers in the one or more samples identifies the individual as one who is at risk for CRC recurrence and may benefit from a treatment comprising an anti-cancer therapy.

Exemplary Embodiment 7: A method of selecting a therapy for an individual at risk for CRC recurrence, the method comprising detecting in one or more samples from the individual one or more biomarkers selected from:
(a) an MSI-H status,
(b) a high TMB,
(c) a PD-L1 positive status, and
(d) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as one who is at risk for CRC recurrence and may benefit from a chemotherapy.

Exemplary Embodiment 8: A method of selecting a therapy for an individual at risk for CRC recurrence, the method comprising detecting in one or more samples from the individual one or more biomarkers selected from:
(a) an MSI-H status,
(b) a high TMB,
(c) a PD-L1 positive status, and
(d) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as one who is at risk for CRC recurrence and may benefit from a chemotherapy.

Exemplary Embodiment 9: A method of selecting a therapy for an individual at risk for CRC recurrence, the method comprising detecting in one or more samples from the individual one or more biomarkers selected from:
(a) an MSI-H status,
(b) a high TMB, and
(c) a PD-L1 positive status; and
wherein detection of the one or more biomarkers in the one or more samples identifies the individual as one who is at risk for CRC recurrence and may benefit from a chemotherapy.

Exemplary Embodiment 10: The method of any one of embodiments 1-9, wherein detection of the one or more biomarkers in the one or more samples identifies the individual as being at greater risk for CRC recurrence as compared to an individual with a CRC that does not comprise the one or more biomarkers.

Exemplary Embodiment 11: A method of identifying one or more treatment options for an individual at risk for CRC recurrence, the method comprising:
(a) detecting in one or more samples from the individual one or more biomarkers selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof; and
(b) generating a report comprising one or more treatment options identified for the individual based, at least in part, on detection of the one or more biomarkers in the one or more samples, wherein the one or more treatment options comprise an anti-cancer therapy, and
wherein the report indicates that the individual is at risk for CRC recurrence based, at least in part, on detection of the one or more biomarkers in the one or more samples.

Exemplary Embodiment 12: A method of identifying one or more treatment options for an individual at risk for CRC recurrence, the method comprising:
(a) detecting in one or more samples from the individual one or more biomarkers selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
(b) generating a report comprising one or more treatment options identified for the individual based, at least in part, on detection of the one or more biomarkers in the one or more samples, wherein the one or more treatment options comprise an anti-cancer therapy, and
wherein the report indicates that the individual is at risk for CRC recurrence based, at least in part, on detection of the one or more biomarkers in the one or more samples.

Exemplary Embodiment 13: A method of identifying one or more treatment options for an individual at risk for CRC recurrence, the method comprising:
(a) detecting in one or more samples from the individual one or more biomarkers selected from:
   (i) an MSI-H status,
   (ii) a high TMB, and
   (iii) a PD-L1 positive status; and
(b) generating a report comprising one or more treatment options identified for the individual based, at least in part, on detection of the one or more biomarkers in the one or more samples, wherein the one or more treatment options comprise an anti-cancer therapy, and
wherein the report indicates that the individual is at risk for CRC recurrence based, at least in part, on detection of the one or more biomarkers in the one or more samples.

Exemplary Embodiment 14: A method of identifying one or more treatment options for an individual at risk for CRC recurrence, the method comprising:
(a) acquiring knowledge of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof; and
(b) generating a report comprising one or more treatment options identified for the individual based at least in part on said knowledge, wherein the one or more treatment options comprise an anti-cancer therapy, and
wherein the report indicates that the individual is at risk for CRC recurrence based, at least in part, on the knowledge of the one or more biomarkers in the one or more samples.

Exemplary Embodiment 15: A method of identifying one or more treatment options for an individual at risk for CRC recurrence, the method comprising:
(a) acquiring knowledge of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
(b) generating a report comprising one or more treatment options identified for the individual based at least in part on said knowledge, wherein the one or more treatment options comprise an anti-cancer therapy, and
wherein the report indicates that the individual is at risk for CRC recurrence based, at least in part, on the knowledge of the one or more biomarkers in the one or more samples.

Exemplary Embodiment 16: A method of identifying one or more treatment options for an individual at risk for CRC recurrence, the method comprising:
(a) acquiring knowledge of one or more biomarkers in one or more samples from the individual, wherein the one or more biomarkers are selected from:
(i) an MSI-H status,
(ii) a high TMB, and
(iii) a PD-L1 positive status; and
(b) generating a report comprising one or more treatment options identified for the individual based at least in part on said knowledge, wherein the one or more treatment options comprise an anti-cancer therapy, and
wherein the report indicates that the individual is at risk for CRC recurrence based, at least in part, on the knowledge of the one or more biomarkers in the one or more samples.

Exemplary Embodiment 17: The method of any one of embodiments 11-16, wherein the report indicates that the individual is at greater risk for CRC recurrence as compared to an individual with a CRC that does not comprise the one or more biomarkers.

Exemplary Embodiment 18: A method of selecting a treatment for an individual at risk for CRC recurrence, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof,
and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

Exemplary Embodiment 19: A method of selecting a treatment for an individual at risk for CRC recurrence, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof;
and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

Exemplary Embodiment 20: A method of selecting a treatment for an individual at risk for CRC recurrence, comprising acquiring knowledge of one or more biomarkers in one or more samples from the individual, wherein the one or more biomarkers are selected from:
(i) an MSI-H status,
(ii) a high TMB, and
(iii) a PD-L1 positive status;
and wherein responsive to the acquisition of said knowledge the individual is classified as being at risk for CRC recurrence, and (i) as a candidate to receive a treatment comprising an anti-cancer therapy, or (ii) as likely to respond to a treatment that comprises an anti-cancer therapy.

Exemplary Embodiment 21: The method of any one of embodiments 18-20, wherein responsive to the acquisition of said knowledge, the individual is classified as being at greater risk for CRC recurrence as compared to an individual with a CRC that does not comprise the one or more biomarkers.

Exemplary Embodiment 22: A method of predicting survival of an individual having a CRC, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 23: A method of predicting survival of an individual having a CRC, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 24: A method of predicting survival of an individual having a CRC, comprising acquiring knowledge of one or more biomarkers in one or more samples from the individual, wherein the one or more biomarkers are selected from:
(i) an MSI-H status,
(ii) a high TMB, and
(iii) a PD-L1 positive status; and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 25: A method of predicting survival of an individual having a CRC treated with a treatment comprising an anti-cancer therapy, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 26: A method of predicting survival of an individual having a CRC treated with a treatment comprising an anti-cancer therapy, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 27: A method of predicting survival of an individual having a CRC treated with a treatment comprising an anti-cancer therapy, comprising acquiring knowledge of one or more biomarkers in one or more samples from the individual, wherein the one or more biomarkers are selected from:
(i) an MSI-H status,
(ii) a high TMB, and
(iii) a PD-L1 positive status; and
wherein responsive to the acquisition of said knowledge, the individual is predicted to be at risk for CRC recurrence and to have longer survival when treated with a treatment comprising an anti-cancer therapy, as compared to survival of an individual with a CRC that does not comprise the one or more biomarkers and/or that is not treated with a treatment comprising said anti-cancer therapy.

Exemplary Embodiment 28: A method of predicting likelihood of recurrence of a CRC in an individual, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein responsive to the acquisition of said knowledge, the individual is predicted to be at risk of CRC recurrence.

Exemplary Embodiment 29: A method of predicting likelihood of recurrence of a CRC in an individual, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein responsive to the acquisition of said knowledge, the individual is predicted to be at risk of CRC recurrence.

Exemplary Embodiment 30: A method of predicting likelihood of recurrence of a CRC in an individual, comprising acquiring knowledge of one or more biomarkers in one or more samples from the individual, wherein the one or more biomarkers are selected from:
(i) an MSI-H status,
(ii) a high TMB, and
(iii) a PD-L1 positive status; and
wherein responsive to the acquisition of said knowledge, the individual is predicted to be at risk of CRC recurrence.

Exemplary Embodiment 31: The method of any one of embodiments 22-30, wherein the individual is predicted to be at greater risk for CRC recurrence as compared to an individual with a CRC that does not comprise the one or more biomarkers.

Exemplary Embodiment 32: A method of treating or delaying progression of a CRC in an individual, comprising:
(a) acquiring knowledge of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof,
   wherein responsive to the acquisition of said knowledge the individual is identified as being at risk for CRC recurrence; and
(b) responsive to said knowledge, administering to the individual an effective amount of a treatment that comprises an anti-cancer therapy.

Exemplary Embodiment 33: A method of treating or delaying progression of a CRC in an individual, comprising:
(a) acquiring knowledge of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof,
wherein responsive to the acquisition of said knowledge the individual is identified as being at risk for CRC recurrence; and
(b) responsive to said knowledge, administering to the individual an effective amount of a treatment that comprises an anti-cancer therapy.

Exemplary Embodiment 34: A method of treating or delaying progression of a CRC in an individual, comprising:
(a) acquiring knowledge of one or more biomarkers in one or more samples from the individual, wherein the one or more biomarkers are selected from:
(i) an MSI-H status,
(ii) a high TMB, and
(iii) a PD-L1 positive status,
wherein responsive to the acquisition of said knowledge the individual is identified as being at risk for CRC recurrence; and
(b) responsive to said knowledge, administering to the individual an effective amount of a treatment that comprises an anti-cancer therapy.

Exemplary Embodiment 35: A method of treating or delaying progression of a CRC in an individual, comprising administering to an individual having a CRC an effective amount of a treatment that comprises an anti-cancer therapy,
wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof.

Exemplary Embodiment 36: A method of treating or delaying progression of a CRC in an individual, comprising administering to an individual having a CRC an effective amount of a treatment that comprises an anti-cancer therapy,
wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof.

Exemplary Embodiment 37: A method of treating or delaying progression of a CRC in an individual, comprising administering to an individual having a CRC an effective amount of a treatment that comprises an anti-cancer therapy,
wherein the anti-cancer therapy is administered to the individual responsive to identifying the individual as being at risk for CRC recurrence based, at least in part, on acquiring knowledge of one or more biomarkers in one or more samples from the individual, wherein the one or more biomarkers are selected from:
(i) an MSI-H status,
(ii) a high TMB, and
(iii) a PD-L1 positive status.

Exemplary Embodiment 38: The method of any one of embodiments 32-37, wherein the individual is identified as being at greater risk for CRC recurrence as compared to an individual with a CRC that does not comprise the one or more biomarkers.

Exemplary Embodiment 39: A method of monitoring, evaluating or screening an individual for CRC recurrence risk, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein responsive to the acquisition of said knowledge, the individual is predicted to have increased risk of CRC recurrence, as compared to an individual with a CRC that does not comprise the one or more biomarkers.

Exemplary Embodiment 40: A method of monitoring, evaluating or screening an individual for CRC recurrence risk, comprising acquiring knowledge of one or more biomarkers in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein responsive to the acquisition of said knowledge, the individual is predicted to have increased risk of CRC recurrence, as compared to an individual with a CRC that does not comprise the one or more biomarkers.

Exemplary Embodiment 41: A method of monitoring, evaluating or screening an individual for CRC recurrence risk, comprising acquiring knowledge of one or more biomarkers in one or more samples from the individual, wherein the one or more biomarkers are selected from:
(i) an MSI-H status,
(ii) a high TMB, and
(iii) a PD-L1 positive status; and
wherein responsive to the acquisition of said knowledge, the individual is predicted to have increased risk of CRC recurrence, as compared to an individual with a CRC that does not comprise the one or more biomarkers.

Exemplary Embodiment 42: A method of assessing one or more biomarkers of CRC recurrence, the method comprising:
(a) detecting one or more biomarkers in one or more samples from an individual having a CRC, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof, and
(b) providing an assessment of the one or more biomarkers in the one or more samples, wherein detecting the one or more biomarkers in the one or more samples from the individual identifies the individual as being at risk for CRC recurrence.

Exemplary Embodiment 43: A method of assessing one or more biomarkers of CRC recurrence, the method comprising:
(a) detecting one or more biomarkers in one or more samples from an individual having a CRC, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
(b) providing an assessment of the one or more biomarkers in the one or more samples, wherein detecting the one or more biomarkers in the one or more samples from the individual identifies the individual as being at risk for CRC recurrence.

Exemplary Embodiment 44: A method of assessing one or more biomarkers of CRC recurrence, the method comprising:
(a) detecting one or more biomarkers in one or more samples from an individual having a CRC, wherein the one or more biomarkers are selected from:
   (i) an MSI-H status,
   (ii) a high TMB, and
   (iii) a PD-L1 positive status; and
(b) providing an assessment of the one or more biomarkers in the one or more samples, wherein detecting the one or more biomarkers in the one or more samples from the individual identifies the individual as being at risk for CRC recurrence.

Exemplary Embodiment 45: A method of detecting the presence or absence of a CRC in an individual, the method comprising:
(a) detecting the presence or absence of a CRC in a sample from the individual; and
(b) detecting the presence or absence of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof,
   wherein detecting the presence of a CRC and the one or more biomarkers in one or more samples from the individual identifies the individual as being at risk for CRC recurrence.

Exemplary Embodiment 46: A method of detecting the presence or absence of a CRC in an individual, the method comprising:
(a) detecting the presence or absence of a CRC in a sample from the individual; and
(b) detecting the presence or absence of one or more biomarkers in one or more samples from the individual, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof,
   wherein detecting the presence of a CRC and the one or more biomarkers in one or more samples from the individual identifies the individual as being at risk for CRC recurrence.

Exemplary Embodiment 47: A method of detecting the presence or absence of a CRC in an individual, the method comprising:
(a) detecting the presence or absence of a CRC in a sample from the individual; and
(b) detecting the presence or absence of one or more biomarkers in one or more samples from the individual, wherein the one or more biomarkers are selected from:
   (i) an MSI-H status,
   (ii) a high TMB, and
   (iii) a PD-L1 positive status,
   wherein detecting the presence of a CRC and the one or more biomarkers in one or more samples from the individual identifies the individual as being at risk for CRC recurrence.

Exemplary Embodiment 48: A method for monitoring recurrence of a CRC in an individual, the method comprising:
(a) detecting, in one or more samples obtained from the individual at a first time point, the presence or absence of one or more biomarkers;
(b) detecting, in one or more samples obtained from the individual at a second time point after the first time point, the presence or absence of one or more biomarkers; and
(c) providing an assessment of CRC recurrence risk in the individual based, at least in part, on the presence or absence of the one or more biomarkers in the one or more samples at the first time point and/or at the second time point;
wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof,
   wherein detecting the presence of the one or more biomarkers in the one or more samples at the first time point and/or at the second time point identifies the individual as being at risk for CRC recurrence.

Exemplary Embodiment 49: A method for monitoring recurrence of a CRC in an individual, the method comprising:
(a) detecting, in one or more samples obtained from the individual at a first time point, the presence or absence of one or more biomarkers;
(b) detecting, in one or more samples obtained from the individual at a second time point after the first time point, the presence or absence of one or more biomarkers; and
(c) providing an assessment of CRC recurrence risk in the individual based, at least in part, on the presence or absence of the one or more biomarkers in the one or more samples at the first time point and/or at the second time point;
wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof,
   wherein detecting the presence of the one or more biomarkers in the one or more samples at the first time point and/or at the second time point identifies the individual as being at risk for CRC recurrence.

Exemplary Embodiment 50: A method for monitoring recurrence of a CRC in an individual, the method comprising:
(a) detecting, in one or more samples obtained from the individual at a first time point, the presence or absence of one or more biomarkers;
(b) detecting, in one or more samples obtained from the individual at a second time point after the first time point, the presence or absence of one or more biomarkers; and
(c) providing an assessment of CRC recurrence risk in the individual based, at least in part, on the presence or absence of the one or more biomarkers in the one or more samples at the first time point and/or at the second time point;
wherein the one or more biomarkers are selected from:
   (i) an MSI-H status,
   (ii) a high TMB, and
   (iii) a PD-L1 positive status,
   wherein detecting the presence of the one or more biomarkers in the one or more samples at the first time point and/or at the second time point identifies the individual as being at risk for CRC recurrence.

Exemplary Embodiment 51: The method of any one of embodiments 48-50, further comprising selecting a treatment, administering a treatment, adjusting a treatment, adjusting a dose of a treatment, or applying a treatment to the individual based, at least in part, on detecting the presence of the one or more biomarkers at the first time point and/or at the second time point, wherein the treatment comprises an anti-cancer therapy.

Exemplary Embodiment 52: A method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from:
(i) an MSI-H status,
(ii) a high TMB, and
(iii) an alteration in one or more genes, wherein the one or more genes comprise BRAF, PTEN, RNF43, ASXL1, CREBBP, MLL2, BCORL1, ATR, or SPEN, or any combination thereof,
wherein identifying the presence of the one or more biomarkers in the one or more samples identifies the individual as being at risk for CRC recurrence,
wherein the candidate treatment comprises an anti-cancer therapy.

Exemplary Embodiment 53: A method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from:
(i) an MSI-H status,
(ii) a high TMB, and
(iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof,
wherein identifying the presence of the one or more biomarkers in the one or more samples identifies the individual as being at risk for CRC recurrence,
wherein the candidate treatment comprises an anti-cancer therapy.

Exemplary Embodiment 54: A method of identifying a candidate treatment for an individual at risk for CRC recurrence, comprising performing DNA sequencing on one or more samples obtained from the individual to determine a sequencing mutation profile, wherein the sequencing mutation profile identifies the presence or absence of one or more biomarkers selected from:
(i) an MSI-H status, and
(ii) a high TMB,
wherein identifying the presence of the one or more biomarkers in the one or more samples identifies the individual as being at risk for CRC recurrence,
wherein the candidate treatment comprises an anti-cancer therapy.

Exemplary Embodiment 55: The method of any one of embodiments 52-54, further comprising detecting the presence or absence of an additional biomarker in sample from the individual.

Exemplary Embodiment 56: The method of embodiment 55, wherein the additional biomarker is a PD-L1 positive status.

Exemplary Embodiment 57: The method of any one of embodiments 55-56, wherein the presence of the additional biomarker in the sample identifies the individual as being at risk for CRC recurrence.

Exemplary Embodiment 58: The method of any one of embodiments 52-57, wherein the presence of the biomarker(s) in one or more samples from the individual identifies the individual as one who may benefit from a treatment comprising an anti-cancer therapy.

Exemplary Embodiment 59: The method of any one of embodiments 52-58, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 60: The method of embodiment 59, wherein the sequencing comprises a massively parallel sequencing technique, and the massively parallel sequencing technique comprises next generation sequencing (NGS).

Exemplary Embodiment 61: A method of treating or delaying progression of a CRC, comprising:
(a) detecting one or more biomarkers in one or more samples from an individual having a CRC, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, or SPEN, or any combination thereof,
   wherein responsive to detecting the one or more biomarkers in the one or more samples, the individual is identified as being at risk for CRC recurrence; and
(b) responsive to detecting the one or more biomarkers in the one or more samples, administering to the individual an effective amount of a treatment that comprises an anti-cancer therapy.

Exemplary Embodiment 62: A method of treating or delaying progression of a CRC, comprising:
(a) detecting one or more biomarkers in one or more samples from an individual having a CRC, wherein the one or more biomarkers are selected from:
   (i) an MSI-H status,
   (ii) a high TMB,
   (iii) a PD-L1 positive status, and
   (iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof,
   wherein responsive to detecting the one or more biomarkers in the one or more samples, the individual is identified as being at risk for CRC recurrence; and
(b) responsive to detecting the one or more biomarkers in the one or more samples, administering to the individual an effective amount of a treatment that comprises an anti-cancer therapy.

Exemplary Embodiment 63: A method of treating or delaying progression of a CRC, comprising:
(a) detecting one or more biomarkers in one or more samples from an individual having a CRC, wherein the one or more biomarkers are selected from:
   (i) an MSI-H status,
   (ii) a high TMB, and
   (iii) a PD-L1 positive status,
   wherein responsive to detecting the one or more biomarkers in the one or more samples, the individual is identified as being at risk for CRC recurrence; and
(b) responsive to detecting the one or more biomarkers in the one or more samples, administering to the individual an effective amount of a treatment that comprises an anti-cancer therapy.

Exemplary Embodiment 64: The method of any one of embodiments 42-63, wherein the individual is identified as being at greater risk for CRC recurrence as compared to an individual with a CRC that does not comprise the one or more biomarkers.

Exemplary Embodiment 65: The method of any one of embodiments 14-41, wherein the acquiring knowledge of the one or more biomarkers in one or more samples from the individual comprises detecting the one or more biomarkers in the one or more samples.

Exemplary Embodiment 66: The method of any one of embodiments 1-65, wherein the CRC is early CRC.

Exemplary Embodiment 67: The method of any one of embodiments 1-65, wherein the CRC is Stage 0, Stage I, Stage II, Stage III, or Stage IV CRC.

Exemplary Embodiment 68: The method of embodiment 67, wherein the CRC is a Stage IV CRC.

Exemplary Embodiment 69: The method of embodiment 68, wherein the CRC comprises an alteration in an APC and/or TP53 gene; optionally wherein:
(a) the alteration in an APC gene comprises one or more of: R1450*, R876*, splice site 835−8A>G, T1556fs*3, E1309fs*4, R213*, R216*, R564*, R283*, R232*, R1114*, S1465fs*3, Q1367*, R805*, R499*, Q1429*, Q1406*, E1379*, Q1338*, E1309*, E1306*, Y935*, R554*, E941*, Q1378*, V1414fs*1, R302*, E1353*, Q1291*, Y935fs*1, S1356*, E1322*, E1408*, Q1303*, E1397*, P1319fs*2, E1464fs*8, E1295*, E1286*, L1488fs*19, Q1294*, N1455fs*18, S1411fs*4, K534*, G1312*, S1495fs*12, S1344*, R1399fs*9, F1491fs*16, S1415fs*4, S1400*, S1346*, S1315*, Q1328*, E1345*, E1317*, T1493fs*14, S1436fs*37, S1421fs*52, K1182*, D1486fs*21, R332*, F1396fs*19, A1492fs*22, splice site 1548+1G>A, S457*, S1545*, S1465fs*9, R640W, S943*, S1501fs*6, Q188*, Q1477*, Q1244*, P1442fs*31, P1440fs*33, P1439fs*34, K670*, I1580fs*70, E763*, E1554*, W699*, W423*, V1452fs*21, S1415fs*8, S1355fs*19, Q767*, Q236*, L1302fs*3, E893*, E1573*, E1544*, Y1376*, T1556fs*9, T1438fs*35, Q1480*, Q1096*, P1443fs*30, P1424fs*49, M1383fs*3, L1488fs*26, H1490fs*24, E225*, E1538fs*5, E1374*, E1353fs*62, E1309fs*6, C1387*, Y1376fs*9, W1049*, S932*, S811*, S770*, S1539*, S1400fs*1, S1389fs*5, S1355fs*60, S1327*, S1200fs*7, R653K, R1314fs*7, Q901*, Q793*, Q695*, Q264*, Q1228*, Q1065*, P1433fs*40, L1489fs*19, K1310fs*11, I1580fs*69, I1311fs*4, G1288*, F814fs*6, E991*, E403fs*51, E1577fs*73, E1552*, E1536*, E1530*, E1461*, E1353fs*21, E1284*, E1155*, D170fs*4, C1410*, A1351fs*3, Y935fs*19, Y796fs*2, Y1376fs*10, Y1075*, W685*, V452fs*7, T1445fs*28, splice site 835−1G>A, splice site 645+1G>A, splice site 1409−5A>G, splice site 1409−2A>G, splice site 1312+1G>A, S596*, S1581fs*69, S1567*, S1495fs*19, S1355fs*20, S1298fs*7, S1282*, S1272*, R904fs*7, R904fs*12, R405*, R2237*, Q886*, Q789*, Q757*, Q445*, Q260*, Q1477fs*30, Q1469*, Q1444*, or Q1256*; and/or
(b) the alteration in a TP53 gene comprises one or more of: R175H, R282W, R273H, R248Q, R273C, G245S, R213*, R196*, R248W, R306*, R342*, C176F, M237L, C238Y, P152L, Y220C, G266E, C141Y, splice site 375G>A, V173L, V173M, C135F, R158H, W146*, C176Y, I195T, C275Y, G244S, H179R, H179Y, H214R, R249S, R337C, T211I, V172F, V272M, E285K, E286K, P151S, P278S, R273L, splice site 673−1G>A, T125M, V272L, C275F, E294*, F113V, G244D, G245D, K132N, P27fs*17, Q104*, splice site 672+1G>T, splice site 782+1G>A, V122fs*26, Y163C, A161T, C135Y, C242F, G266R, L257P, L257Q, N131fs*27, P151H, P153fs*28, S166*, S215G, S215N, splice site 375+1G>T, splice site 559+1G>A, V216M, V73fs*76, C238F, C242fs*5, C242Y, E171*, E204*, E258G, E271K, G244A, G266*, H178fs*3, H193Y, K132R, L194F, N239fs*9, P250L, P278T, Q165*, R110L, R209fs*6, R213L, R213Q, S127F, S215L, splice site 672G>A, splice site 782+1G>T, splice site 88_96+1delAACGTTCTGG, V274F, Y126C, Y126N, Y205D, Y234C, Y236C, A159V, A276G, C135R, C135W, C141R, C141W, C277F, D259Y, E198*, E258*, E271*, E298*, E51*, F134L, F270C, G244C, G266V, H193L, L130F, L130V, L194P, L35fs*9, MIT, N247L, P191del, Q167*, Q192*, Q317*, R181H, R181P, R249M, S214W, S241fs*6, S261fs*85, S90fs*33, S90fs*59, splice site 375+5G>T, splice site 375G>C, splice site 376−1G>A, splice site 560−1G>A, splice site 560−1G>T, splice site 560−2A>T, splice site 560−3T>G, splice site 673−1G>T, splice site 920−2A>G, splice site 993+1G>A, V157F, V216L, V274A, V274G, W91*, Y163H, Y205H, Y234H, Y236fs*14, Y236H, C124fs*25, C141*, C182fs*65, C229*, C238R, D393fs*78, E180*, E198fs*49, E221*, E224D, E258A, E258K, E285*, E336fs*4, E339*, E349*, F109V, F134C, F134V, or F270L.

Exemplary Embodiment 70: The method of embodiment 67, wherein the CRC is Stage I, Stage II, or Stage III CRC.

Exemplary Embodiment 71: The method of embodiment 70, wherein the CRC comprises an alteration in one or more of a BRCA1, BRCA2, MSH6, MLH1, and/or MSH2 gene, optionally wherein:
(a) the alteration in a BRCA1 gene comprises one or more of: K339fs*2, K654fs*47, Q1756fs*74, Y655fs*18, A224fs*4, E181*, E577*, E732*, H1686R, K1711fs*3, L1098fs*4, L63F, Q74*, R1203*, R1495M, R1751*, S1457*, S324fs*16, splice site 4185+2_4185+22>A, splice site 442−2A>G, splice site 5277+1G>A, or V340fs*6;
(b) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*;
(c) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H;
(d) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G; and/or
(e) the alteration in a MSH2 gene comprises one or more of: A230fs*16, S233fs*13, R680*, E580*, E480*, Y408*, splice site 943−1G>C, splice site 942+3A>T, R406*, R389*, Q61*, Q574*, Q324*, L634*, L277fs*5, L187R, K449fs*5, I134fs*8, G683R, E86fs*4, E850*, E188*, C778fs*35, or C778fs*34.

Exemplary Embodiment 72: The method of embodiment 70, wherein the CRC comprises an alteration in one or more of a RNF43, MLL2, MSH3, PTCH1, CDK12, ARID1A, ASXL1, MSH6, BCORL1, CTNNB1, MLH1, CIC, MAP3K1, ATR, MSH2, CTCF, JAK1, QKI, CDH1, CASP8, NOTCH3, EP300, BRCA2, MEN1, or BCOR gene, or any combination thereof, optionally wherein:
(a) the alteration in a RNF43 gene comprises one or more of: G659fs*41, R117fs*41, R225fs*194, R117fs*8, R145*, P660fs*41, P660fs*87, R371*, R132*, R330*, R337*, A273fs*147, E37fs*11, K181fs*4, S216L, V479fs*25, Y248*, Y332*, A169T, A193fs*6, A78T, C290*, E258fs*162, E318*, E37*, F103fs*20, G257fs*162, G29*, G659fs*87, H352fs*87, I48T, K60fs*2, L311fs*108, L311fs*132, L53fs*1, L82*, L88fs*13, M1I, M55fs*7, N167fs*1, P370fs*49, P715fs*15, P77fs*18, Q153*, Q233*, Q254*, Q283*, Q426*, Q426fs*77, Q6fs*29, Q8*, R113*, R225fs*195, R286W, R49fs*3, S607L, S687fs*13, splice site 375+1G>A, splice site 583−177_592del187, splice site 687+1G>A, splice site 688−1G>A, splice site 850−2A>G, splice site 952+2T>C, T158fs*10, V271fs*11, V271fs*149, V299fs*120, V490fs*12, W13*, W13fs*26, W159*, W165*, W200*, W302*, Y332fs*110, or Y332fs*11;
(b) the alteration in a MLL2 gene comprises one or more of: P2354fs*30, G1235fs*95, P647fs*283, P648fs*2, R4904*, A1390fs*27, Q836fs*94, A2119fs*25, C2436fs*49, C346fs*17, G5182fs*61, H1497fs*30, R2443fs*6, R4238C, R845fs*3, T382fs*20, T4629fs*11, V1244fs*86, V4799M, A1390fs*42, A2169T, A2205fs*59, A221fs*40, A3552fs*4, C5123*, C5142fs*5, D2769N, E2962fs*42, F1790fs*12, F2494fs*49, F2566fs*17, G1317*, G1960fs*87, G1995*, G2262fs*37, G2265fs*21, G3189*, G3698fs*51, H77fs*53, I4491fs*1, I977fs*23, K1686fs*36, K304fs*30, K3140fs*2, K4843fs*15, L1020fs*36, L1271fs*15, L2331fs*46, L2594fs*97, L3716fs*296, L3880fs*131, L5183fs*16, L5318fs*14, P1460fs*46, P2206fs*58, P2382fs*2, P367fs*35, P4380fs*4, P444fs*2, P4968fs*27, P506fs*424, P583fs*347, P62fs*9, P886fs*44, Q1377R, Q1557*, Q211*, Q3471*, Q3811fs*201, Q3839fs*42, Q3909fs*103, Q3934*, Q3950R, Q4235fs*98, Q4284*, Q791fs*139, Q809fs*121, Q809fs*3, R1252*, R1687fs*4, R2099*, R2471*, R2771*, R2830*, R4198*, R466C, R5048H, R5086*, R5120C, R5282*, R5454*, R755fs*3, S102fs*28, S1107fs*12, S1684fs*38, S1684T, S2532fs*11, S2910fs*32, S3159fs*16, S4010fs*12, S4507fs*12, S456*, S4789fs*27, splice site 14644−1G>T, splice site 16413−2A>G, T209fs*11, T2191fs*11, T698fs*232, V1670fs*52, V3089fs*30, Y2199fs*65, or Y2907fs*3;
(c) the alteration in a MSH3 gene comprises one or more of: K383fs*32, L564fs*1, E342*, K902fs*5, K99fs*3, N1020fs*17, N385fs*19, N524fs*3, N739fs*8, N861fs*6, or R268*;
(d) the alteration in a PTCH1 gene comprises one or more of: S1203fs*52, R1308fs*64, Y1316fs*56, L39fs*41, R1308fs*17, E61fs*18, N97fs*43, V1164I, A563V, C1398fs*54, C727fs*19, E1242K, G526*, L50fs*39, N97fs*20, P643fs*11, R602*, or R6fs*1;
(e) the alteration in a CDK12 gene comprises one or more of: G1461fs*38, Q1291fs*3, T1463fs*50, G1271fs*23, R983*, E59fs*33, E751*, E887*, H1035fs*7, 1873fs*11, K445*, L342fs*8, L996*, N474fs*8, N864fs*2, P683fs*70, P686fs*13, P974L, Q115*, Q1418*, R1048*, R1331*, R298*, R890H, S133fs*24, splice site 1047−2A>C, T1346fs*7, or T212fs*18;
(f) the alteration in a ARID1A gene comprises one or more of: D1850fs*33, D1850fs*4, F2141fs*59, G276fs*87, P1326fs*155, P224fs*8, Q766fs*67, K1072fs*21, Q1452fs*29, Q372fs*19, Q758fs*75, Q802fs*15, R1989*, A339fs*24, M1634fs*14, Q372fs*28, R693*, Y551fs*72, G314fs*49, P1115fs*46, P1568fs*44, P1898fs*25, Q1200*, Q1519fs*8, Q1631*, R1501fs*4, S1000Y, S11fs*91, W1073fs*32, A134fs*98, A1539fs*27, A27fs*24, A339fs*61, A62fs*39, A77fs*24, D1850fs*34, D2178fs*47, D2260fs*5, E1297*, E1733*, E1783fs*6, E2058*, E2120*, E992*, G1110fs*51, G122fs*278, G126fs*274, G1740*, G2069fs*50, G2087R, G236fs*163, G277fs*86, G285fs*78, G37fs*14, G801fs*32, G82fs*19, G83fs*28, G987fs*50, K1094fs*67, K1905fs*18, K250*, L1049fs*55, L1841fs*2, L2082fs*53, L2238fs*30, L2270fs*8, M1154fs*7, M1273fs*10, M1318fs*163, M1388fs*94, M1564fs*8, M1595fs*19, M1634fs*1, M890fs*46, N1313fs*168, N2109fs*26, P1175fs*5, P1451fs*41, P1468fs*13, P146fs*86, P1560fs*5, P225fs*175, P469fs*150, Q1188*, Q1212*, Q1250*, Q1327*, Q1327fs*11, Q1420*, Q1512*, Q1584*, Q1650*, Q1708*, Q1835*, Q1835fs*1, Q1974*, Q2115*, Q2176*, Q2176fs*48, Q288*, Q505fs*117, Q521*, Q538*, Q546fs*73, Q566*, Q575fs*46, Q581*, Q611*, Q633*, Q806fs*11, R1223C, R1335*, R1446*, R1461*, R1658fs*40, R1658W, R1722*, R1869fs*30, S1465fs*25, S1645fs*46, S2264*, S255fs*145, S366fs*25, S536fs*87, S617fs*2, S617fs*6, splice site 1921−3_1925delTAGGATCT, splice site 2879−2A>G, splice site 3715+1G>C, splice site 4005−2A>T, T1514fs*13, T1743M, T2252fs*27, T286fs*114, T894fs*25, V1561fs*11, V63fs*38, W1545*, W1670*, W2050*, W2091*, Y1101fs*1, Y1377*, Y2076*, Y222*, Y422*, or Y551fs*68;
(g) the alteration in a ASXL1 gene comprises one or more of: G645fs*58, G646fs*12, R693*, E635fs*15, G646fs*58, Q592*, A627fs*8, E41K, E518*, E566*, E676*, E917*, F354L, G1376fs*74, G643fs*15, G643fs*61, G967del, L983fs*8, N1158fs*6, P1377fs*3, P763fs*12, P808fs*10, Q561fs*1, Q588*, Q695*, Q768fs*6, R541fs*162, R596fs*107, R718fs*7, S1335fs*115, S747fs*25, S892fs*16, splice site 140+2T>G, splice site 471+1G>A, T957fs*26, V737fs*10, W1037*, or W583*;
(h) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H;
(i) the alteration in a BCORL1 gene comprises one or more of: P1681fs*20, A1166fs*56, G1682fs*4, A74fs*42, M644fs*4, A858fs*67, Q1001fs*49, R1299*, S803fs*83, A74fs*20, A971fs*4, E1655*, E619*, I389fs*29, K1207N, K1330fs*17, L275fs*143, N1412fs*38, P323fs*95, Q459*, R1196*, R1297*, R1338*, R1420*, R609*, R743fs*13, splice site 4306−2A>G, or W1105*;
(j) the alteration in a CTNNB1 gene comprises one or more of: S45F, T41A, S45P, Q773*, R587*, S33C, T41I, D32N, E334K, G34E, N387K, R449C, S45A, T257I, W25*, W383R, D17_Q78del, D17_T75del, E568*, G69*, I35_G38del, I35S, K19_S37>N, K335fs*10, K335L, M8_V79del, R376H, R515Q, R582Q, R582W, R90*, R95*, S33F, S33T, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242−39del684, splice site 14−338_81>TTAC, splice site 14−39_225del251, splice site 14−48_241+12del288, splice site 14−5_97del89, splice site 14−6_241+74del308, splice site 14−69_242−10del488, splice site 14−7_89del83, splice site 14−80_241+22>CAT, splice site 14−91_241+25del344, splice site 1954+1G>A, splice site 1954+1G>T, splice site 1955−1G>A, splice site 2138−2A>C, splice site 241+1G>C, splice site 32_241+94del304, splice site 60_287del428, splice site 65_274del410, splice site 74_241+52del220, splice site 78_241+11del175, splice site 87_242−79del277, splice site 98_241+8>A, V22_S33del, V22_S37del, W25_I35>C, W383C, or W776*;
(k) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G;
(l) the alteration in a CIC gene comprises one or more of: P1597fs*23, P1248fs*54, P509fs*14, P1116fs*45, T1375fs*40, P135fs*70, P1598fs*16, S1117fs*34, S961fs*6, A785fs*139, A900fs*24, D449fs*23, E367*, G136fs*8, G1600fs*14, P1128fs*33, P1336fs*3, P1529fs*91, P404fs*31, P515fs*8, P518fs*5, P574fs*154, P768fs*156, P911fs*13, P98fs*107, Q378*, R1313W, R201W, R353*, S902fs*21, S904fs*27, splice site 583−1G>A, T1541fs*18, T1541fs*79, or T328fs*78;
(m) the alteration in a MAP3K1 gene comprises one or more of: C635*, E126*, E1293fs*3, E788*, G1074fs*8, G608fs*48, H114fs*50, K1160fs*12, L380fs*4, L915*, N1212fs*33, P74fs*3, Q1022*, Q320*, R208*, R288*, R307fs*5, R532*, S101fs*63, splice site 3983−1G>A, splice site 483−1G>A, splice site 633+2T>A, T1145fs*6, T457fs*31, V1045fs*12, or V569I;
(n) the alteration in a ATR gene comprises one or more of: I774fs*5, R1814fs*10, F1091fs*28, F1134fs*6, I774fs*3, E148*, E1699*, E2579*, F2168*, F222fs*11, I1264fs*14, I691fs*5, I774fs*6, K446fs*11, K773fs*3, L1029fs*20, Q195*, R1015Q, R1814fs*8, R2001*, R223fs*1, R224fs*18, R2533*, R2547*, R2598*, S2207fs*15, S825fs*13, splice site 5381−1G>A, W1591*, or Y1844*;
(o) the alteration in a MSH2 gene comprises one or more of: A230fs*16, C778fs*34, E188*, E86fs*4, R680*, S233fs*13, or Y408*;
(p) the alteration in a CTCF gene comprises one or more of: T204fs*26, T204fs*18, R166C, E363fs*5, A137fs*17, A225V, D194fs*28, E112*, E145*, E182fs*9, G32fs*30, H19fs*15, MIT, N259fs*44, P50L, R11W, R275C, splice site 374−1G>T, splice site 854−1G>T, T317fs*91, Y15H, or Y195*;
(q) the alteration in a JAK1 gene comprises one or more of: K860fs*16, P430fs*2, G741D, or K496N;
(r) the alteration in a QKI gene comprises one or more of: K134fs*14, A313V, E42*, G77fs*14, L236fs*54, R319*, splice site 1010−233_*194del444, splice site 142+1G>A, splice site 143−1G>T, or splice site 546+1G>T;
(s) the alteration in a CDH1 gene comprises one or more of: P126fs*89, P127fs*41, R492fs*44, S70fs*13, A241fs*3, A634V, C28*, D257G, D291N, D400G, G169fs*46, L214P, L711V, P372fs*8, Q16*, R335*, R63*, R74*, S111fs*6, S18fs*39, splice site 1137G>A, splice site 1138−1G>A, splice site 1565+1G>A, splice site 1711+2T>C, splice site 688−1G>T, T323fs*33, T340M, or W532*;
(t) the alteration in a CASP8 gene comprises one or more of: R449*, K490fs*73, F373fs*26, I350fs*4, E212*, R452*, A197fs*14, P411L, R194fs*17, R68*, Y252*, A264fs*24, C196Y, D380fs*19, D380fs*2, E195*, E36*, F152fs*18, F296fs*11, GI IR, K478fs*10, K478fs*19, L59fs*12, L62P, N475fs*13, R250W, splice site 1355+2T>C, splice site 151+1G>A, V222fs*13, V268fs*8, V492fs*71, or Y8fs*1;
(u) the alteration in a NOTCH3 gene comprises one or more of: A1802fs*8, C1344fs*76, P695fs*165, C43fs*32, G1318fs*245, G2035fs*50, P1317fs*103, T250fs*122, A1020fs*252, A1927T, C720fs*1, C87fs*149, D352fs*2, E1492fs*84, G2035fs*60, G2081fs*4, G707D, K2069fs*16, L2092fs*57, P2033fs*62, P2115fs*10, P42fs*194, R1589Q, R2031fs*54, R6fs*28, S1448fs*115, S157fs*5, splice site 119-156_197+47del282, splice site 1606+1G>A, splice site 5668−1G>T, T1098fs*174, or W1425*;
(v) the alteration in a EP300 gene comprises one or more of: H2324fs*55, M1470fs*26, R1187H, splice site 1282+1G>A, C1385F, D1399N, H2324fs*29, L2303fs*74, L415P, R86*, C1408*, C1738*, E643fs*2, G54*, K1469fs*3, M1339fs*26, N1236fs*41, N419fs*12, Q2282*, Q498*, Q501fs*6, R1055*, R1281*, R1312*, R1627W, R2185*, R2263*, R2330fs*49, R580Q, R648*, S1214Y, S19fs*19, S2271fs*8, splice site 1169−2A>G, splice site 2379+1G>C, splice site 3671+1G>A, splice site 4453−2A>G, T1021fs*3, or Y1467H;
(w) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*;
(x) the alteration in a MEN1 gene comprises one or more of: R521fs*43, E184V, L105fs*13, R457Q, R521fs*15, R532*, or splice site 799−9G>A; and/or
(y) the alteration in a BCOR gene comprises one or more of: P1587fs*53, Q1174fs*8, K1173fs*31, G400fs*42, R810*, Q1272fs*20, R1480*, S158fs*28, S336fs*45, C1329fs*45, D328fs*50, E1025*, E1030fs*48, E1182fs*6, E485fs*42, G154*, G906fs*5, G95fs*16, H1179fs*1, 11290fs*2, K1061fs*52, K1137fs*4, K1271fs*64, K1330*, K839fs*17, L279fs*21, N1425S, N390fs*53, N529fs*28, P602fs*67, P931fs*15, Q1174*, Q1274*, Q348*, Q430*, Q600*, R1053fs*26, R1181fs*1, R1498fs*36, R976*, S1371L, S336fs*42, splice site 166−2A>C, splice site 2997+1G>T, splice site 3239−2A>G, splice site 4072−2A>G, T1331fs*4, or V806fs*10.

Exemplary Embodiment 73: The method of embodiment 70, wherein the CRC is microsatellite stable (MSS) and/or does not comprise a POLE and/or POLD1 alteration, and wherein the CRC comprises an alteration in a CTNNB1 and/or MAP3K1 gene, optionally wherein:
(a) the alteration in a CTNNB1 gene comprises one or more of: T41A, S45F, Q773*, R587*, S33C, D32N, N387K, S45P, T257L, W25*, D17_Q78del, D17_T75del, I35_G38del, I35S, K19_S37>N, K335I, M8_V79del, R376H, R582Q, R582W, R90*, R95*, S33F, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242−39del684, splice site 14−338_81>TTAC, splice site 14−39_225del251, splice site 14−48_241+12del288, splice site 14−6_241+74del308, splice site 14−69_242−10del488, splice site 14−7_89del83, splice site 14−80_241+22>CAT, splice site 14−91_241+25del344, splice site 1954+1G>A, splice site 1954+1G>T, splice site 1955−1G>A, splice site 241+1G>C, splice site 32_241+94del304, splice site 60_287del428, splice site 65_274del410, splice site 74_241+52del220, splice site 78_241+11del175, splice site 87_242−79del277, splice site 98_241+8>A, T41I, V22_S33del, V22_S37del, W25_I35>C, W383C, W383R, or W776*; and/or
(b) the alteration in a MAP3K1 gene comprises one or more of: E126*, E1293fs*3, E788*, H114fs*50, L380fs*4, P74fs*3, Q1022*, R532*, S101fs*63, splice site 483−1G>A, T1145fs*6, or T457fs*31.

Exemplary Embodiment 74: The method of any one of embodiments 1-73, wherein the CRC is a primary CRC or is metastatic.

Exemplary Embodiment 75: The method of any one of embodiments 1-74, wherein the CRC comprises one or more KRAS alterations, optionally wherein the one or more KRAS alterations comprise a G12C, G12D, G12V, or G13D amino acid substitution.

Exemplary Embodiment 76: The method of any one of embodiments 1-75, wherein the CRC is mismatch repair deficient (dMMR).

Exemplary Embodiment 77: The method of any one of embodiments 1-75, wherein the CRC is not dMMR CRC.

Exemplary Embodiment 78: The method of any one of embodiments 1-77, wherein the CRC is an adenocarcinoma, a carcinoid tumor, a familial CRC, a gastrointestinal stromal tumor (GIST), a colorectal lymphoma, a squamous cell carcinoma, a leiomyosarcoma, or an angiosarcoma.

Exemplary Embodiment 79: The method of any one of embodiments 1-78, wherein the one or more biomarkers comprise an MSI-H status.

Exemplary Embodiment 80: The method of any one of embodiments 1-13, 17, 42-51, and 61-79, wherein the MSI-H status is detected by sequencing, a polymerase chain reaction (PCR) amplification technique, a non-PCR amplification technique, an isothermal amplification technique, a capillary electrophoresis method, immunohistochemistry, or any combination thereof.

Exemplary Embodiment 81: The method of embodiment 80, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 82: The method of embodiment 81, wherein the sequencing comprises a massively parallel sequencing technique, and the massively parallel sequencing technique comprises next-generation sequencing (NGS).

Exemplary Embodiment 83: The method of any one of embodiments 1-13, 17, 42-51, and 61-82, wherein the MSI-H status is detected based on DNA sequencing of up to about 114 loci; and/or wherein the MSI-H status is determined if at least about 1.24% of analyzed loci have instability.

Exemplary Embodiment 84: The method of any one of embodiments 1-83, wherein the one or more biomarkers comprise a high TMB.

Exemplary Embodiment 85: The method of any one of embodiments 1-84, wherein the high TMB comprises a TMB of ≥10 mutations/Megabase (mut/Mb).

Exemplary Embodiment 86: The method of any one of embodiments 1-13, 17, 42-51, and 61-85, wherein the high TMB is detected based on about 0.79 megabases (Mb) of sequenced DNA.

Exemplary Embodiment 87: The method of any one of embodiments 1-13, 17, 42-51, and 61-85, wherein the high TMB is detected based on about 0.80 Mb of sequenced DNA.

Exemplary Embodiment 88: The method of any one of embodiments 1-13, 17, 42-51, and 61-85, wherein the high TMB is detected on between about 0.83 Mb and about 1.14 Mb of sequenced DNA, or between about 0.8 Mb and about 1.1 Mb of sequenced DNA.

Exemplary Embodiment 89: The method of any one of embodiments 1-13, 17, 42-51, and 61-85, wherein the high TMB is detected based on about 1.1 Mb of sequenced DNA.

Exemplary Embodiment 90: The method of any one of embodiments 1-13, 17, 42-51, and 61-85, wherein the high TMB is detected based on up to about 1.24 Mb of sequenced DNA.

Exemplary Embodiment 91: The method of any one of embodiments 1-13, 17, 42-51, and 61-85, wherein the high TMB is detected based on up to about 1.1 Mb of sequenced DNA.

Exemplary Embodiment 92: The method of any one of embodiments 1-91, wherein the high TMB comprises a TMB of at least about 10 mut/Mb, at least about 15 mut/Mb, at least about 20 mut/Mb, at least about 25 mut/Mb, at least about 30 mut/Mb, at least about 35 mut/Mb, at least about 40 mut/Mb, at least about 45 mut/Mb, at least about 50 mut/Mb, at least about 55 mut/Mb, at least about 60 mut/Mb, at least about 65 mut/Mb, at least about 70 mut/Mb, at least about 75 mut/Mb, at least about 80 mut/Mb, at least about 85 mut/Mb, at least about 90 mut/Mb, at least about 95 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

Exemplary Embodiment 93: The method of any one of embodiments 1-13, 17, 42-51, and 61-92, wherein the high TMB is detected by sequencing, whole exome sequencing, whole genome sequencing, gene-targeted sequencing, or next-generation sequencing.

Exemplary Embodiment 94: The method of any one of embodiments 1-51 and 56-93, wherein the one or more biomarkers comprise a PD-L1 positive status.

Exemplary Embodiment 95: The method of any one of embodiments 1-13, 17, 42-51, and 61-94, wherein the PD-L1 positive status is detected based on PD-L1 protein expression.

Exemplary Embodiment 96: The method of embodiment 95, wherein PD-L1 protein expression is determined using an immunohistochemistry assay.

Exemplary Embodiment 97: The method of embodiment 96, wherein the immunohistochemistry assay is a DAKO PD-L1 22C3 assay.

Exemplary Embodiment 98: The method of embodiment 96 or embodiment 97, wherein PD-L1 expression is assessed based on a tumor proportion score (TPS).

Exemplary Embodiment 99: The method of embodiment 98, wherein the PD-L1 positive status comprises a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%.

Exemplary Embodiment 100: The method of embodiment 99, wherein the PD-L1 positive status comprises a TPS of between about 1% and about 49%.

Exemplary Embodiment 101: The method of embodiment 99, wherein the PD-L1 positive status comprises a TPS of at least about 1%.

Exemplary Embodiment 102: The method of embodiment 99, wherein the PD-L1 positive status comprises a TPS of at least about 25%, at least about 50%, or at least about 75%.

Exemplary Embodiment 103: The method of embodiment 96 or embodiment 97, wherein PD-L1 expression is assessed based on a combined positive score (CPS).

Exemplary Embodiment 104: The method of embodiment 103, wherein the PD-L1 positive status comprises a CPS of at least about 1 or at least about 10.

Exemplary Embodiment 105: The method of embodiment 96, wherein the immunohistochemistry assay is a VENTANA SP 142 assay.

Exemplary Embodiment 106: The method of embodiment 96 or embodiment 105, wherein PD-L1 expression is assessed based on the proportion of tumor area occupied by PD-L1-expressing tumor-infiltrating immune cells of any intensity (IC), or the percentage of PD-L1-expressing tumor cells of any intensity (TC).

Exemplary Embodiment 107: The method of embodiment 105 or embodiment 106, wherein the CRC is PD-L1 positive.

Exemplary Embodiment 108: The method of embodiment 106 or embodiment 107, wherein the PD-L1 positive status comprises a TC or IC of at least about 1%.

Exemplary Embodiment 109: The method of any one of embodiments 1-2, 4-5, 7-8, 10-12, 14-15, 17-19, 21-23, 25-26, 28-29, 31-33, 35-36, 38-40, 42-43, 45-46, 48-49, 51-53, 55-62, and 64-108, wherein the one or more biomarkers comprise an alteration in the one or more genes.

Exemplary Embodiment 110: The method of any one of embodiments 1-2, 4-5, 7-8, 10-12, 14-15, 17-19, 21-23, 25-26, 28-29, 31-33, 35-36, 38-40, 42-43, 45-46, 48-49, 51-53, 55-62, and 64-109, wherein the alteration in the one or more genes comprises a point mutation; an insertion-deletion (indel); an in-frame deletion of one or more codons; an intragenic deletion; an intragenic insertion; a deletion of a full gene; an inversion; an interchromosomal or intrachromosomal translocation; a tandem duplication; a gene fusion; a genomic rearrangement; a splice site mutation; and/or a gene amplification or duplication.

Exemplary Embodiment 111: The method of any one of embodiments 1-2, 4-5, 7-8, 10-12, 14-15, 17-19, 21-23, 25-26, 28-29, 31-33, 35-36, 38-40, 42-43, 45-46, 48-49, 51-53, 55-62, and 64-110, wherein:
(a) the alteration in BRAF is a V600E, D594G, G469A, N581S, G466V, K483E, L485F, L485S, or T241M alteration, or any combination thereof,
(b) the alteration in PTEN is a K267fs*9, N323fs*21, R233*, R130*, R130Q, E299*, R173H, T319fs*1, C136Y, C250fs*2, D24fs*20, E157fs*23, E242fs*9, F90fs*9, H93R, I33del, K164fs*3, K330*, L247*, L325P, L57fs*6, N323fs*2, N340fs*3, N63fs*11, P95L, Q171*, Q219*, Q245*, Q261*, R234W, S59*, splice site 209+5G>A, splice site 210−1G>C, splice site 626_634+2delGAACTTGCAGT, splice site 79+1G>A, T131N, or V133I alteration, or any combination thereof;
(c) the alteration in RNF43 is a G659fs*41, R117fs*41, R225fs*194, R132*, R145*, R330*, Y332*, A193fs*6, A273fs*147, A78T, E258fs*162, G257fs*162, K181fs*4, L311fs*132, M1I, P660fs*87, Q153*, Q233*, Q426*, Q426fs*77, Q8*, R225fs*195, R337*, splice site 375+1G>A, V271fs*11, V299fs*120, V479fs*25, W159*, W302*, Y248*, or Y332fs*110V alteration, or any combination thereof;
(d) the alteration in ASXL1 is a G645fs*58, G646fs*12, R693*, A627fs*8, E41K, E676*, G646fs*58, L983fs*8, P1377fs*3, P763fs*12, Q561fs*1, or S892fs*16 alteration, or any combination thereof;
(e) the alteration in CREBBP is a I1084fs*15, P1423fs*36, A1824T, Q1209fs*25, G1145fs*23, R714H, I1084fs*3, K668fs*27, L555fs*7, P2094L, P937fs*61, Q278*, Q911*, R1446H, S801*, splice site 3836+1G>A, or Y1503H alteration, or any combination thereof,
(f) the alteration in MLL2 is a P2354fs*30 9, G1235fs*95, P647fs*283, T382fs*20, A2205fs*59, C2436fs*49, C346fs*17, D2769N, E2962fs*42, F1790fs*12, G2265fs*21, H77fs*53, I977fs*23, K1686fs*36, K304fs*30, L1020fs*36, L5183fs*16, P1460fs*46, P2206fs*58, P367fs*35, P4380fs*4, P444fs*2, P4968fs*27, P506fs*424, P583fs*347, P648fs*2, Q1377R, Q3811fs*201, R1252*, R1687fs*4, R2771*, R2830*, R4238C, R4904*, R5048H, R5282*, R755fs*3, S1107fs*12, S1684T, S2910fs*32, S4507fs*12, S4789fs*27, splice site 14644−1G>T, splice site 16413−2A>G, T209fs*11, V1244fs*86, V1670fs*52, or V4799M alteration, or any combination thereof;
(g) the alteration in BCORL1 is a P1681fs*20, A1166fs*56, A971fs*4, E1655*, E619*, G1682fs*4, K1207N, P323fs*95, Q1001fs*49, R1297*, R1299*, R1420*, or W1105* alteration, or any combination thereof;
(h) the alteration in ATR is a I774fs*5, F1091fs*28, F1134fs*6, I774fs*3, E2579*, F2168*, I691fs*5, K446fs*11, K773fs*3, R2001*, R223fs*1, R2547*, or W1591* alteration, or any combination thereof;
(i) the alteration in SPEN is a A2105fs*33, R806fs*14, A2105fs*18, H2985fs*199, I1052fs*40, I577fs*37, N2002fs*20, P2495fs*4, P2839fs*50, P3631fs*3, Q253fs*109, R1936*, R2332H, or V1294fs*7 alteration, or any combination thereof,
(j) the alteration in BRCA1 is a K654fs*47, Q1756fs*74, Q74*, R1203*, S324fs*16, splice site 4185+2_4185+22>A, or splice site 442−2A>G alteration, or any combination thereof,
(k) the alteration in BRCA2 is a E2981K, E2981fs*7, N1784fs*7, R2842C, T3033fs*29, A1237fs*2, C3233fs*15, D252fs*24, E2144*, E3316fs*2, E597*, E866*, M2393fs*19, N1287fs*6, N1784fs*3, N2189fs*2, R2034H, R2318*, T3085fs*26, V1862fs*1, or W2830* alteration, or any combination thereof, and/or
(l) the alteration in MSH6 is a F1088fs*5, F1088fs*2, F1088fs*3, E946*, A1236fs*4, A1320fs*5, C694fs*4, K1140fs*24, K247fs*32, L1356fs*1, R240*, R248fs*8, R298*, or R361H alteration, or any combination thereof.

Exemplary Embodiment 112: The method of any one of embodiments 1-111, wherein the CRC is microsatellite stable (MSS).

Exemplary Embodiment 113: The method of any one of embodiments 1-112, wherein the CRC does not have an alteration in a POLE and/or POLD1 gene.

Exemplary Embodiment 114: The method of embodiment 112 or embodiment 113, wherein the one or more genes comprise one or more of BRAF, PTEN, or RNF43; optionally wherein:
(a) the alteration in BRAF is a V600E, D594G, N581S, G466V, G469A, K483E, L485F, or L485S alteration, or any combination thereof;
(b) the alteration in PTEN is a R233*, C136Y, D24fs*20, E242fs*9, E299*, H93R, K330*, L247*, L57fs*6, N323fs*2, N340fs*3, N63fs*11, P95L, Q171*, Q219*, Q245*, R130*, R130Q, splice site 209+5G>A, splice site 210−1G>C, splice site 626_634+2delGAACTTGCAGT, T131N, or V133I alteration, or any combination thereof, and/or
(c) the alteration in RNF43 is a Y332*, A193fs*6, A273fs*147, E258fs*162, MII, P660fs*87, Q233*, Q426fs*77, Q8*, R117fs*41, R145*, R337*, splice site 375+1G>A, V271fs*11, W159*, W302*, Y248*, or Y332fs*110 alteration, or any combination thereof.

Exemplary Embodiment 115: The method of any one of embodiments 1-2, 4-5, 7-8, 10-12, 17, 42-43, 45-46, 48-49, 51, 61-62, and 64-114, wherein the alteration in the one or more genes is detected by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Embodiment 116: The method of embodiment 115, wherein the sequencing comprises a massively parallel sequencing (MPS) technique, whole genome sequencing (WGS), whole exome sequencing, targeted sequencing, direct sequencing, or a Sanger sequencing technique.

Exemplary Embodiment 117: The method of embodiment 116, wherein the massively parallel sequencing (MPS) technique comprises next-generation sequencing (NGS).

Exemplary Embodiment 118: The method of any one of embodiments 1-2, 4-5, 7-8, 10-12, 14-15, 17-19, 21-23, 25-26, 28-29, 31-33, 35-36, 38-40, 42-43, 45-46, 48-49, 51-53, 55-62, and 64-117, further comprising selectively enriching for one or more nucleic acid molecules in a sample from the individual comprising nucleotide sequences corresponding to the one or more genes; wherein the selectively enriching produces an enriched sample.

Exemplary Embodiment 119: The method of embodiment 118, wherein the selectively enriching comprises: (a) combining one or more bait molecules with the sample, thereby hybridizing the one or more bait molecules to one or more nucleic acids in the sample comprising nucleotide sequences corresponding to the one or more genes and producing nucleic acid hybrids; and (b) isolating the nucleic acid hybrids to produce the enriched sample.

Exemplary Embodiment 120: The method of embodiment 118, wherein the selectively enriching comprises amplifying one or more nucleic acid molecules in a sample from the individual comprising nucleotide sequences corresponding to the one or more genes in the sample; thereby producing the enriched sample.

Exemplary Embodiment 121: The method of any one of embodiments 118-120, further comprising sequencing the enriched sample to detect the alteration in the one or more genes.

Exemplary Embodiment 122: The method of any one of embodiments 1-2, 4-5, 7-8, 10-12, 14-15, 17-19, 21-23, 25-26, 28-29, 31-33, 35-36, 38-40, 42-43, 45-46, 48-49, 51-53, 55-62, and 64-117, further comprising:
(a) optionally, ligating one or more adapters onto nucleic acid molecules in a sample from the individual, thereby generating ligated nucleic acids;
(b) optionally, amplifying nucleic acids from the ligated nucleic acids;
(c) optionally, capturing or enriching from the amplified nucleic acids a plurality of nucleic acids comprising nucleotide sequences corresponding to the one or more genes; (d) sequencing, by a sequencer, the plurality of nucleic acids to obtain a plurality of sequence reads corresponding to the one or more genes;
(e) analyzing the plurality of sequence reads; and
(f) based on the analysis, detecting the alteration in the one or more genes.

Exemplary Embodiment 123: The method of embodiment 122, wherein the plurality of nucleic acids comprising nucleotide sequences corresponding to the one or more genes is captured from the amplified nucleic acids by hybridization with one or more bait molecules.

Exemplary Embodiment 124: The method of embodiment 123, wherein the capturing comprises: (a) combining the one or more bait molecules with the amplified nucleic acids, thereby hybridizing the one or more bait molecules to nucleic acids comprising nucleotide sequences corresponding to the one or more genes and producing nucleic acid hybrids; and (b) isolating the nucleic acid hybrids.

Exemplary Embodiment 125: The method of any one of embodiments 122-124, further comprising receiving, at one or more processors, sequence read data for the plurality of sequence reads.

Exemplary Embodiment 126: The method of embodiment 125, wherein the analyzing the plurality of sequence reads comprises identifying, using the one or more processors, the presence or absence of sequence reads corresponding to the one or more genes and/or the alteration in the one or more genes.

Exemplary Embodiment 127: The method of any one of embodiments 122-126, wherein the one or more adapters comprise amplification primers, flow cell adapter sequences, substrate adapter sequences, sample index sequences, or unique molecular identifier (UMI) sequences.

Exemplary Embodiment 128: The method of any one of embodiments 122-127, wherein the sequencer comprises a next generation sequencer.

Exemplary Embodiment 129: The method of any one of embodiments 120-128, wherein the amplifying comprises performing a polymerase chain reaction (PCR) amplification technique, a non-PCR amplification technique, or an isothermal amplification technique.

Exemplary Embodiment 130: The method of any one of embodiments 121-129, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 131: The method of embodiment 130, wherein the sequencing comprises a massively parallel sequencing technique, and the massively parallel sequencing technique comprises next generation sequencing (NGS).

Exemplary Embodiment 132: The method of any one of embodiments 1-131, further comprising generating a molecular profile for the individual, based, at least in part, on detecting or acquiring knowledge of the one or more biomarkers in the one or more samples from the individual.

Exemplary Embodiment 133: The method of embodiment 132, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 134: The method of embodiment 132 or embodiment 133, wherein the molecular profile for the individual further comprises results from a nucleic acid sequencing-based test.

Exemplary Embodiment 135: The method of any one of embodiments 132-134, further comprising selecting a treatment, administering a treatment, or applying a treatment to the individual based on the generated molecular profile.

Exemplary Embodiment 136: The method of any one of embodiments 1-135, comprising generating a report indicating the presence or absence of the one or more biomarkers in one or more samples from the individual.

Exemplary Embodiment 137: The method of any one of embodiments 125-136, comprising generating, by the one or more processors, a report indicating the presence or absence of the alteration in the one or more genes in a sample from the individual.

Exemplary Embodiment 138: The method of any one of embodiments 11-17 and 65-137, further comprising transmitting the report to the individual, a caregiver, a healthcare provider, a physician, an oncologist, an electronic medical record system, a hospital, a clinic, a third-party payer, an insurance company, or a government office.

Exemplary Embodiment 139: The method of embodiment 138, wherein the report is transmitted via a computer network or a peer-to-peer connection.

Exemplary Embodiment 140: The method of any one of embodiments 119, 121, and 123-139, wherein the one or more bait molecules comprise a capture nucleic acid molecule configured to hybridize to a nucleotide sequence corresponding to the one or more genes.

Exemplary Embodiment 141: The method of embodiment 140, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Embodiment 142: The method of any one of embodiments 119, 121, and 123-141, wherein the one or more bait molecules are conjugated to an affinity reagent and/or to a detection reagent.

Exemplary Embodiment 143: The method of embodiment 142, wherein the affinity reagent is an antibody, an antibody fragment, or biotin, and/or wherein the detection reagent is a fluorescent marker.

Exemplary Embodiment 144: The method of any one of embodiments 140-143, wherein the capture nucleic acid molecule comprises a DNA, RNA, or mixed DNA/RNA molecule.

Exemplary Embodiment 145: The method of any one of embodiments 1-144, wherein detecting, acquiring knowledge of, or identifying the presence of the one or more biomarkers in the one or more samples from the individual identifies or predicts the individual as being at risk for early CRC recurrence.

Exemplary Embodiment 146: The method of embodiment 145, wherein detecting, acquiring knowledge of, or identifying the presence of the one or more biomarkers in the one or more samples from the individual identifies or predicts the individual as being at greater risk for early CRC recurrence, as compared to an individual with a CRC that does not comprise the one or more biomarkers.

Exemplary Embodiment 147: The method of embodiment 145 or embodiment 146, wherein detecting, acquiring knowledge of, or identifying the presence of the one or more biomarkers in the one or more samples from the individual identifies or predicts the individual as being at risk for CRC recurrence within about one year or less from the time of initial diagnosis of the CRC.

Exemplary Embodiment 148: The method of embodiment 147, wherein detecting, acquiring knowledge of, or identifying the presence of the one or more biomarkers in the one or more samples from the individual identifies or predicts the individual as being at greater risk for CRC recurrence within about one year or less from the time of initial diagnosis of the CRC, as compared to an individual with a CRC that does not comprise the one or more biomarkers.

Exemplary Embodiment 149: The method of any one of embodiments 1-31, 39-50, 52-60, and 64-148, further comprising administering an anti-cancer therapy to the individual based, at least in part, on detecting, acquiring knowledge, or identifying the presence of the one or more biomarkers in the one or more samples from the individual.

Exemplary Embodiment 150: The method of any one of embodiments 7-10 and 66-149, wherein the chemotherapy comprises 5-fluorouracil (5-FU), irinotecan, oxaliplatin, capecitabine, or Trifluridine/Tipiracil, or any combination thereof; and/or wherein the method further comprises administering the chemotherapy to the individual.

Exemplary Embodiment 151: The method of any one of embodiments 1-150, wherein detecting, acquiring knowledge of, or identifying the presence of the one or more biomarkers in the one or more samples from the individual identifies the individual as one who should be monitored or assessed for CRC recurrence more frequently, as compared to an individual with a CRC that does not comprise the one or more biomarkers.

Exemplary Embodiment 152: The method of any one of embodiments 1-151, wherein detecting, acquiring knowledge of, or identifying the presence of the one or more biomarkers in the one or more samples from the individual identifies the individual as a candidate to receive a more aggressive anti-cancer therapy for CRC and/or an anti-cancer therapy for CRC of greater duration, as compared to an individual with a CRC that does not comprise the one or more biomarkers.

Exemplary Embodiment 153: The method of any one of embodiments 1-152, wherein detecting, acquiring knowledge of, or identifying the presence of the one or more biomarkers in the one or more samples from the individual identifies the individual as one who: (a) should be administered a standard-of-care treatment for CRC, optionally a more aggressive standard-of-care treatment for CRC and/or a standard-of-care treatment for CRC of greater duration, as compared to an individual with a CRC that does not comprise the one or more biomarkers; or (b) should be administered an anti-cancer therapy other than a standard-of-care treatment for CRC, or an anti-cancer therapy combined with a standard-of-care treatment for CRC.

Exemplary Embodiment 154: The method of embodiment 153, wherein the standard-of-care treatment for CRC comprises a chemotherapy combined with an anti-VEGF agent or an anti-EGFR agent.

Exemplary Embodiment 155: The method of any one of embodiments 7-10 and 66-154, wherein the chemotherapy comprises a leucovorin calcium (folinic acid), fluorouracil, and oxaliplatin combination (FOLFOX).

Exemplary Embodiment 156: The method of embodiment 154 or embodiment 155, wherein the anti-VEGF agent is an anti-VEGF antibody, optionally wherein the anti-VEGF antibody is bevacizumab.

Exemplary Embodiment 157: The method of any one of embodiments 154-156, wherein the anti-EGFR agent is an anti-EGFR antibody, optionally wherein the anti-EGFR antibody is cetuximab.

Exemplary Embodiment 158: The method of any one of embodiments 1-6, 10-27, 31-38, 51-149 and 151-157, wherein the anti-cancer therapy comprises a chemotherapy, a radiation therapy, an immunotherapy, a targeted therapy, a surgery, or any combination thereof, optionally wherein the chemotherapy comprises 5-fluorouracil (5-FU), irinotecan, oxaliplatin, capecitabine, or Trifluridine/Tipiracil, or any combination thereof.

Exemplary Embodiment 159: The method of any one of embodiments 1-6, 10-27, 31-38, 51-149 and 151-158, wherein the anti-cancer therapy comprises a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), a tyrosine kinase inhibitor therapy, or any combination thereof.

Exemplary Embodiment 160: The method of embodiment 159, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 161: The method of embodiment 159, wherein the nucleic acid comprises a double-stranded RNA (dsRNA), a small interfering RNA (siRNA), or a small hairpin RNA (shRNA).

Exemplary Embodiment 162: The method of any one of embodiments 1-6, 10-27, 31-38, 51-149 and 151-161, wherein the one or more biomarkers comprise an MSI-H status, a high TMB, and/or a PD-L1 positive status, and wherein the anti-cancer therapy comprises an immunotherapy.

Exemplary Embodiment 163: The method of any one of embodiments 1-2, 4-5, 10-12, 14-15, 17-19, 21-23, 25-26, 31-33, 35-36, 38, 51-53, 55-62, 64-149 and 151-162, wherein:
the one or more biomarkers comprise an alteration in a BRAF gene, and wherein the anti-cancer therapy comprises a BRAF-targeted therapy, an EGFR-targeted therapy, or a combination thereof,
the one or more biomarkers comprise an alteration in a PTEN gene, and wherein the anti-cancer therapy comprises a PTEN-targeted therapy;
the one or more biomarkers comprise an alteration in a RNF43 gene, and wherein the anti-cancer therapy comprises a RNF43-targeted therapy, a BRAF-targeted therapy, an EGFR-targeted therapy, a MEK-targeted therapy, or a combination thereof,
the one or more biomarkers comprise an alteration in an ASXL1 gene, and wherein the anti-cancer therapy comprises an ASXL1-targeted therapy;
the one or more biomarkers comprise an alteration in a CREBBP gene, and wherein the anti-cancer therapy comprises a CREBBP-targeted therapy;
the one or more biomarkers comprise an alteration in an MLL2 gene, and wherein the anti-cancer therapy comprises an MLL2-targeted therapy;
the one or more biomarkers comprise an alteration in a BCORL1 gene, and wherein the anti-cancer therapy comprises a BCORL1-targeted therapy;
the one or more biomarkers comprise an alteration in an ATR gene, and wherein the anti-cancer therapy comprises an ATR-targeted therapy;
the one or more biomarkers comprise an alteration in a SPEN gene, and wherein the anti-cancer therapy comprises a SPEN-targeted therapy;
the one or more biomarkers comprise an alteration in a BRCA1 gene, and wherein the anti-cancer therapy comprises a BRCA1-targeted therapy;
the one or more biomarkers comprise an alteration in a BRCA2 gene, and wherein the anti-cancer therapy comprises a BRCA2-targeted therapy; and/or
the one or more biomarkers comprise an alteration in a MSH6 gene, and wherein the anti-cancer therapy comprises a MSH6-targeted therapy.

Exemplary Embodiment 164: The method of embodiment 163, wherein the BRAF-targeted therapy comprises an anti-EGFR agent or a combination of a chemotherapy and an anti-EGFR agent, optionally wherein the anti-EGFR agent is an anti-EGFR antibody.

Exemplary Embodiment 165: The method of any one of embodiments 1-6, 10-27, 31-38, 51-149 and 151-164, wherein the anti-cancer therapy is a first-line anti-cancer therapy for the CRC.

Exemplary Embodiment 166: The method of any one of embodiments 1-165, further comprising obtaining the one or more samples from the individual.

Exemplary Embodiment 167: The method of embodiment 166, wherein one or more of the samples from the individual are obtained about 3 months or less from the time of diagnosis of a CRC in the individual.

Exemplary Embodiment 168: The method of embodiment 166 or embodiment 167, wherein one or more of the samples from the individual are obtained prior to the individual having received a treatment for the CRC.

Exemplary Embodiment 169: The method of any one of embodiments 1-168, wherein one or more of the samples from the individual comprise or are derived from a tissue biopsy sample, a liquid biopsy sample, or a normal control.

Exemplary Embodiment 170: The method of embodiment 169, wherein one or more of the samples from the individual are or are derived from a liquid biopsy sample and comprise blood, plasma, cerebrospinal fluid, sputum, stool, urine, or saliva.

Exemplary Embodiment 171: The method of embodiment 169, wherein one or more of the samples from the individual are a liquid biopsy and comprise circulating tumor cells (CTCs).

Exemplary Embodiment 172: The method of embodiment 169, wherein one or more of the samples from the individual are a liquid biopsy sample and comprise cell-free DNA (cfDNA), circulating tumor DNA (ctDNA), or any combination thereof.

Exemplary Embodiment 173: The method of any one of embodiments 1-172, wherein one or more of the samples from the individual comprise a mixture of tumor nucleic acid molecules and non-tumor nucleic acid molecules.

Exemplary Embodiment 174: The method of embodiment 173, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Exemplary Embodiment 175: The method of embodiment 173, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Exemplary Embodiment 176: An anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as being at risk for CRC recurrence.

Exemplary Embodiment 177: An anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as being at risk for CRC recurrence.

Exemplary Embodiment 178: An anti-cancer therapy for use in a method for treating or delaying progression of a CRC in an individual at risk for CRC recurrence, wherein the method comprises administering the anti-cancer therapy to an individual having a CRC, wherein one or more biomarkers are detected in one or more samples from the individual, wherein the one or more biomarkers are selected from:
(i) an MSI-H status,
(ii) a high TMB, and
(iii) a PD-L1 positive status; and
wherein detection of the one or more biomarkers in the one or more samples identifies the individual as being at risk for CRC recurrence.

Exemplary Embodiment 179: A chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as being at risk for CRC recurrence.

Exemplary Embodiment 180: A chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as being at risk for CRC recurrence.

Exemplary Embodiment 181: A chemotherapy for use in a method for treating or delaying progression of a CRC in an individual at risk for CRC recurrence, wherein the method comprises administering the chemotherapy to an individual having a CRC, wherein one or more biomarkers are detected in one or more samples from the individual, wherein the one or more biomarkers are selected from:
(i) an MSI-H status,
(ii) a high TMB, and
(iii) a PD-L1 positive status; and
wherein detection of the one or more biomarkers in the one or more samples identifies the individual as being at risk for CRC recurrence.

Exemplary Embodiment 182: An anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as being at risk for CRC recurrence.

Exemplary Embodiment 183: An anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as being at risk for CRC recurrence.

Exemplary Embodiment 184: An anti-cancer therapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB, and
(iii) a PD-L1 positive status; and
wherein detection of the one or more biomarkers in the one or more samples identifies the individual as being at risk for CRC recurrence.

Exemplary Embodiment 185: A chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, or SPEN, or any combination thereof, and
wherein detection of the one or more biomarkers in the one or more samples identifies the individual as being at risk for CRC recurrence.

Exemplary Embodiment 186: A chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB,
(iii) a PD-L1 positive status, and
(iv) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
wherein detection of the one or more biomarkers in the one or more samples identifies the individual as being at risk for CRC recurrence.

Exemplary Embodiment 187: A chemotherapy for use in the manufacture of a medicament for treating or delaying progression of a CRC in an individual at risk for CRC recurrence, wherein one or more biomarkers are detected in one or more samples from the individual, wherein the one or more biomarkers are selected from:
(i) an MSI-H status,
(ii) a high TMB, and
(iii) a PD-L1 positive status; and
wherein detection of the one or more biomarkers in the one or more samples identifies the individual as being at risk for CRC recurrence.

Exemplary Embodiment 188: A system, comprising:
a memory configured to store one or more program instructions, and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from one or more samples obtained from an individual having a CRC;
   (b) analyze the plurality of sequence reads for the presence of one or more biomarkers, wherein the one or more biomarkers are selected from:
      (i) an MSI-H status,
      (ii) a high TMB, and
      (iii) an alteration in one or more genes, wherein the one or more genes comprise BRAF, PTEN, RNF43, ASXL1, CREBBP, MLL2, BCORL1, ATR, or SPEN, or any combination thereof; and
   (c) detect, based on the analyzing, the one or more biomarkers in the one or more samples, wherein detection of the one or more biomarkers in the one or more samples identifies the individual as being at risk for CRC recurrence.

Exemplary Embodiment 189: A system, comprising:
a memory configured to store one or more program instructions, and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from one or more samples obtained from an individual having a CRC;
   (b) analyze the plurality of sequence reads for the presence of one or more biomarkers, wherein the one or more biomarkers are selected from:
      (i) an MSI-H status,
      (ii) a high TMB, and
      (iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof; and
   (c) detect, based on the analyzing, the one or more biomarkers in the one or more samples, wherein detection of the one or more biomarkers in the one or more samples identifies the individual as being at risk for CRC recurrence.

Exemplary Embodiment 190: A system, comprising:
a memory configured to store one or more program instructions, and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from one or more samples obtained from an individual having a CRC;
   (b) analyze the plurality of sequence reads for the presence of one or more biomarkers, wherein the one or more biomarkers are selected from:
      (i) an MSI-H status, and
      (ii) a high TMB; and
   (c) detect, based on the analyzing, the one or more biomarkers in the one or more samples, wherein detection of the one or more biomarkers in the one or more samples identifies the individual as being at risk for CRC recurrence.

Exemplary Embodiment 191: A non-transitory computer readable storage medium comprising one or more programs executable by one or more computer processors for performing a method, the method comprising:
(a) obtaining, using the one or more processors, a plurality of sequence reads of one or more nucleic acid molecules, wherein the one or more nucleic acid molecules are derived from one or more samples obtained from an individual having a CRC;
(b) analyzing, using the one or more processors, the plurality of sequence reads for the presence of one or more biomarkers, wherein the one or more biomarkers are selected from:
   (i) an MSI-H status,
   (ii) a high TMB, and
   (iii) an alteration in one or more genes, wherein the one or more genes comprise BRAF, PTEN, RNF43, ASXL1, CREBBP, MLL2, BCORL1, ATR, or SPEN, or any combination thereof; and
(c) detecting, using the one or more processors and based on the analyzing, the one or more biomarkers in the one or more samples, wherein detection of the one or more biomarkers in the one or more samples identifies the individual as being at risk for CRC recurrence.

Exemplary Embodiment 192: A non-transitory computer readable storage medium comprising one or more programs executable by one or more computer processors for performing a method, the method comprising:
(a) obtaining, using the one or more processors, a plurality of sequence reads of one or more nucleic acid molecules, wherein the one or more nucleic acid molecules are derived from one or more samples obtained from an individual having a CRC;
(b) analyzing, using the one or more processors, the plurality of sequence reads for the presence of one or more biomarkers, wherein the one or more biomarkers are selected from:
   (i) an MSI-H status,
   (ii) a high TMB, and
   (iii) an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof, and
(c) detecting, using the one or more processors and based on the analyzing, the one or more biomarkers in the one or more samples, wherein detection of the one or more biomarkers in the one or more samples identifies the individual as being at risk for CRC recurrence.

Exemplary Embodiment 193: A non-transitory computer readable storage medium comprising one or more programs executable by one or more computer processors for performing a method, the method comprising:
(a) obtaining, using the one or more processors, a plurality of sequence reads of one or more nucleic acid molecules, wherein the one or more nucleic acid molecules are derived from one or more samples obtained from an individual having a CRC;
(b) analyzing, using the one or more processors, the plurality of sequence reads for the presence of one or more biomarkers, wherein the one or more biomarkers are selected from:
   (i) an MSI-H status, and
   (ii) a high TMB; and
(c) detecting, using the one or more processors and based on the analyzing, the one or more biomarkers in the one or more samples, wherein detection of the one or more biomarkers in the one or more samples identifies the individual as being at risk for CRC recurrence.

Exemplary Embodiment 194: The system of any one of embodiments 188-190, or the non-transitory computer readable storage medium of any one of embodiments 191-193, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and further optionally wherein the massively parallel sequencing technique comprises next generation sequencing (NGS).

Exemplary Embodiment 195: The system of any one of embodiments 188-190 and 194, wherein the one or more program instructions when executed by the one or more processors are further configured to generate, based at least in part on the detecting, a molecular profile for the sample.

Exemplary Embodiment 196: The non-transitory computer readable storage medium of any one of embodiments 191-194, wherein the method further comprises generating, based at least in part on the detecting, a molecular profile for the sample.

Exemplary Embodiment 197: The system of embodiment 195, or the non-transitory computer readable storage medium of embodiment 196, wherein the individual is administered a treatment based at least in part on the molecular profile; optionally wherein the treatment comprises an anti-cancer therapy.

Exemplary Embodiment 198: The system of embodiment 195 or embodiment 197, or the non-transitory computer readable storage medium of 196 or embodiment 197, wherein the molecular profile further comprises results from a comprehensive genomic profiling (CGP) test, a gene expression profiling test, a cancer hotspot panel test, a DNA methylation test, a DNA fragmentation test, an RNA fragmentation test, a PD-L1 expression test, or any combination thereof.

Exemplary Embodiment 199: The system of any one of embodiments 195 and 197-198, or the non-transitory computer readable storage medium of any one of embodiments 196-198, wherein the molecular profile further comprises results from a nucleic acid sequencing-based test.

Exemplary Embodiment 200: The system of any one of embodiments 195 and 197-199, or the non-transitory computer readable storage medium of any one of embodiments 196-199, wherein the molecular profile further indicates the presence or absence of a PD-L1 positive status in a sample from the individual.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Identification of Biomarkers of Cancer Recurrence in Colorectal Cancer Patients

This Example describes the identification of biomarkers of colorectal cancer (CRC) recurrence risk.

### Methods

### Study Cohort

All patients included in this study had a confirmed diagnosis of metastatic CRC (mCRC), and underwent tissue genomic testing using comprehensive genomic profiling (CGP) assays. Retrospective de-identified longitudinal clinical data were derived from electronic health records (EHR) from approximately 280 US cancer clinics (˜800 sites of care), comprising patient-level structured and unstructured data, curated via technology-enabled abstraction of clinical notes and radiology/pathology reports. These were linked to genomic data by de-identified, deterministic matching. For numeric variable comparison between groups (e.g., age at diagnosis), Kruskal-Wallis test was used. Chi-squared test was used for categorical variables (e.g., tumor grade) and genomic alterations. P values were corrected with the Benjamini-Hochberg FDR method.

### Comprehensive Genomic Profiling (CGP)

CGP on 0.8-1.1 megabases (Mb) of the coding genome was performed on hybridization-captured, adapter-ligation based libraries, to identify genomic alterations (base substitutions, small insertions/deletions, copy number alterations and rearrangements) in exons and select introns, tumor mutational burden (TMB), and microsatellite instability (MSI) status. TMB was calculated as the number of non-driver somatic coding mutations per megabase of genome sequenced (e.g., Chalmers, Z. R., et al. (2017) Genome Med. 9(1):34). MSI status was determined by analyzing 114 intronic homopolymer repeat loci for length variability, and MSI high (MSI-H) was defined as previously described (e.g., Trabucco, S. E., et al. (2019) J. Mol. Diagnostics 21(6):1053-1066). See, e.g., Frampton et al (2013) Nat Biotechnol, 31:1023-1031.

### PD-L1 Expression

PD-L1 immunohistochemistry (IHC) testing was performed according to the manufacturer's instructions (DAKO, PD-L1 IHC 22C3 pharmDx package insert, accessible at www.accessdata.fda.gov/cdrh_docs/pdf15/P150013c.pdf). The tumor proportion score (TPS) method was used to score PD-L1 expression. TPS was defined as the number of PD-L1-positive tumor cells over the total number of tumor cells (PD-L1-positive and PD-L1-negative tumor cells).

### Results

### Cohort Selection and Patient Characteristics

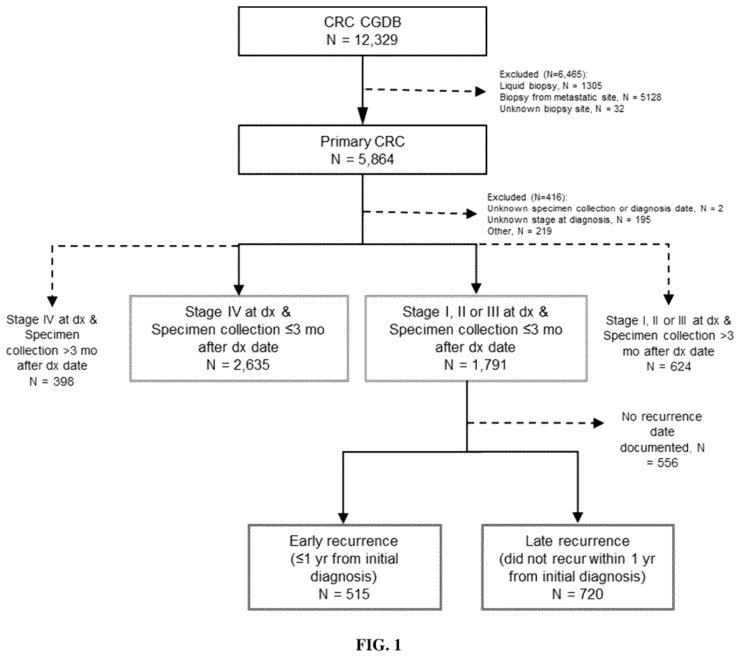

FIG. 1 is a flowchart of the cohort selection process for CRC patients in this study. As depicted in FIG. 1, several exclusion criteria were applied to a starting cohort of 12,329 CRC patients, resulting in 2,635 Stage IV patients and 1,791 Stage I-III patients. Of the Stage I-III patients, 515 had an early recurrence of CRC (i.e., 1 year or less from initial diagnosis), and 720 did not recur within 1 year from initial diagnosis (late recurrence).

### Comparison of Stage I-III and Stage IV CRC

Table 1 provides an overview of the characteristics of patients with Stage IV and Stage I-III CRC. Stage I-III patients were older at diagnosis (p-value<0.001), with a median age at diagnosis of 62 years, compared to Stage IV CRC patients whose median age at diagnosis was 60 years. Furthermore, Stage I-III CRC patients had a higher prevalence of microsatellite instability-high (MSI-H, p-value<0.001) and tumor mutational burden (TMB) ³10 mutations per Mb (p-value<0.001).

TABLE 1
Characteristics of patients with stage I-III and stage IV CRC.

| | Stage I-III | | Stage IV | | |
|---|---|---|---|---|---|
| | (n = 1,791) | | (n = 2,635) | | P-value |
| Age at diagnosis, | 62.0 | (52.0, 72.0) | 60.0 | (51.0, 69.0) | <0.001 |
| Median (IQR) | | | | | |
| Gender (Male) | 962 | (53.7%) | 1434 | (54.4%) | 0.643 |
| Predicted ancestry | | | | | 0.0775 |
| AFR | 217 | (12.1%) | 325 | (12.3%) | |
| AMR | 176 | (9.8%) | 216 | (8.2%) | |
| EAS | 68 | (3.8%) | 82 | (3.1%) | |
| EUR | 1311 | (73.2%) | 1996 | (75.7%) | |
| SAS | 19 | (1.1%) | 16 | (0.6%) | |
| Biopsy site | | | | | 0.311 |
| Colon | 1436 | (80.2%) | 2087 | (79.2%) | |
| Colorectal not | 8 | (0.4%) | 21 | (0.8%) | |
| otherwise | | | | | |
| specified (NOS) | | | | | |
| Rectum | 347 | (19.4%) | 527 | (20.0%) | |
| PD-L1 positive | 83 | (16.6%) | 105 | (16.2%) | 0.869 |
| (DAKO 22C3), | | | | | |
| tumor proportion | | | | | |
| score (TPS) ≥1 | | | | | |
| TMB | 3.8 | (1.7, 6.3) | 3.5 | (1.7, 5.0) | <0.001 |
| TMB ≥10(≥10) | 257 | (14.3%) | 177 | (6.7%) | <0.001 |
| MSI-H (MSI-H) | 204 | (11.4%) | 118 | (4.5%) | <0.001 |

Table 2 shows the number and proportion of MSI-H patients for each CRC stage. Higher proportions of patients at earlier stages of disease were MSI-H.

TABLE 2
MSI status by CRC stage.

| | |
|---|---|
| MSI-H n(%) | Total (n) |

| | | | |
|---|---|---|---|
| Stage I | 10 | (12.6%) | 79 |
| Stage II | 52 | (12.1%) | 428 |
| Stage III | 142 | (11.1%) | 1,284 |
| Stage IV | 118 | (4.5%) | 2,635 |

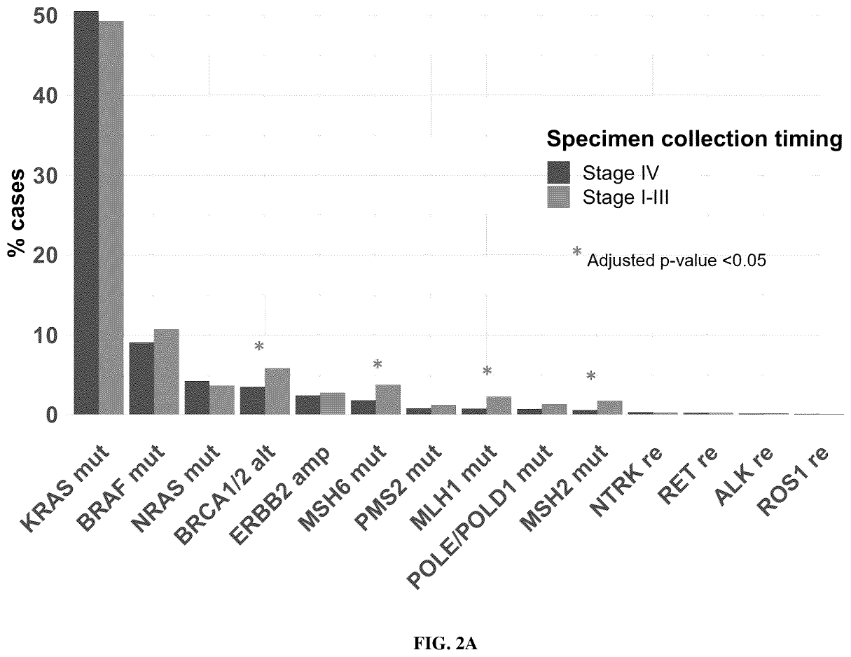

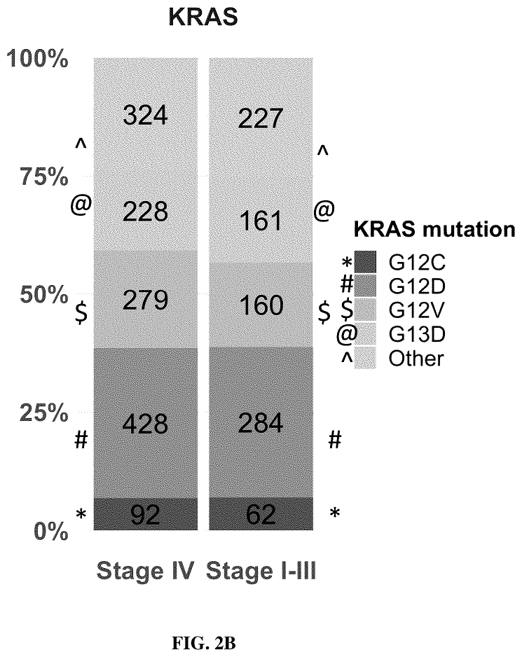

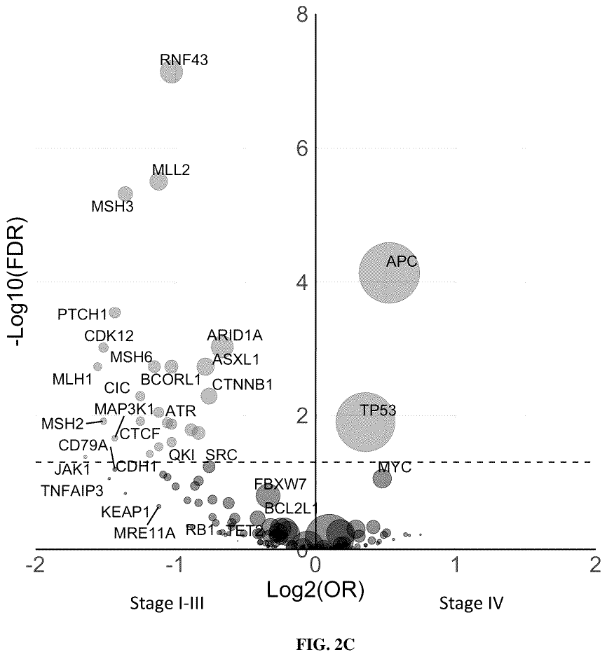

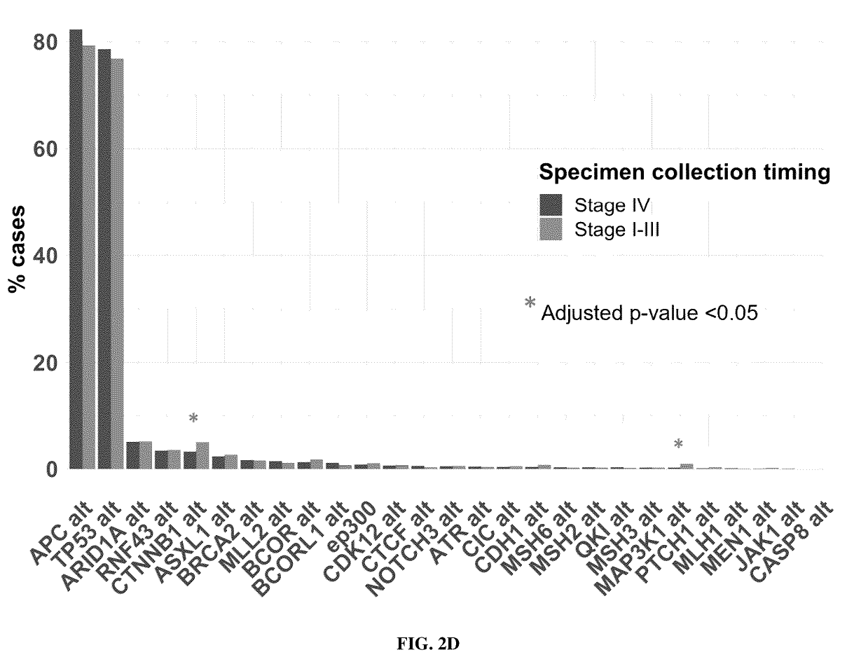

Next, genomic alterations in patients diagnosed with Stage I-III or Stage IV CRC were compared. FIG. 2A shows that among the actionable alterations identified, BRCA1/2, MSH6, MLH1, and MSH2 mutations were enriched in Stage I-III patients (p-value<0.05). FIG. 2B shows the prevalence of G12C, G12D, G12V, G13D, and other KRAS mutations among patients with Stage I-III and Stage IV CRC. FIG. 2C is a volcano plot showing alterations identified in Stage I-III and Stage IV CRC. Alterations associated with Stage I-III CRC included RNF43, MLL2, MSH3, PTCH1, CDK12, ARID1A, ASXL1, MSH6, BCORL1, CTNNB1, MLH1, CIC, MAP3K1, ATR, MSH2, CTCF, JAK1, QKI, CDH1, CASP8, NOTCH3, EP300, BRCA2, MEN1, and BCOR. Meanwhile, alterations in APC and TP53 were associated with Stage IV disease. The prevalence of gene alterations in patients with microsatellite stability (MSS) and no POLE/POLDI mutations was also investigated. As shown in FIG. 2D, alterations in CTNNB1 and MAP3K1 were found to be enriched in Stage I-III disease that was MSS and had no POLE/POLD1 mutations.

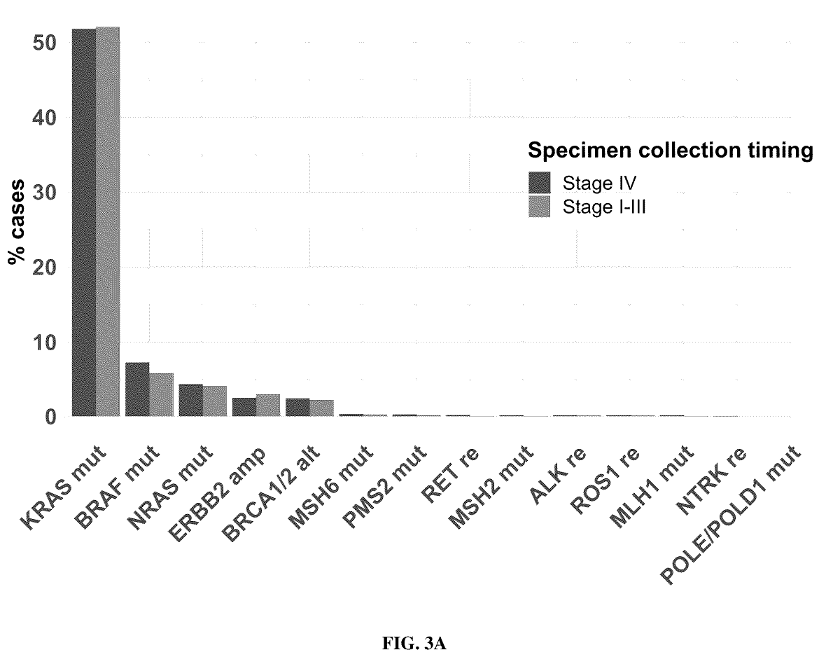

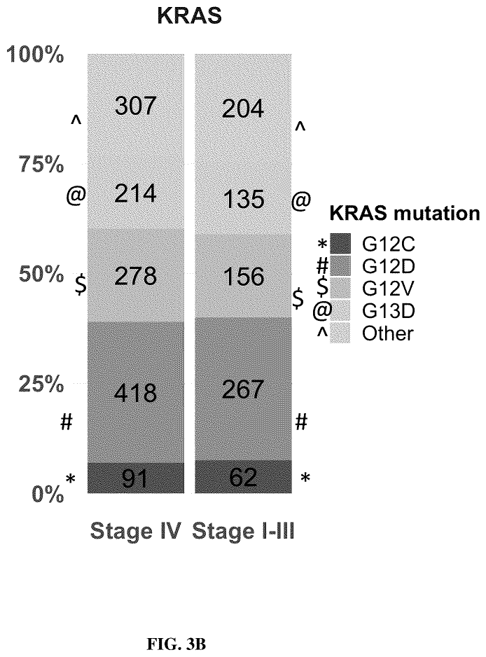

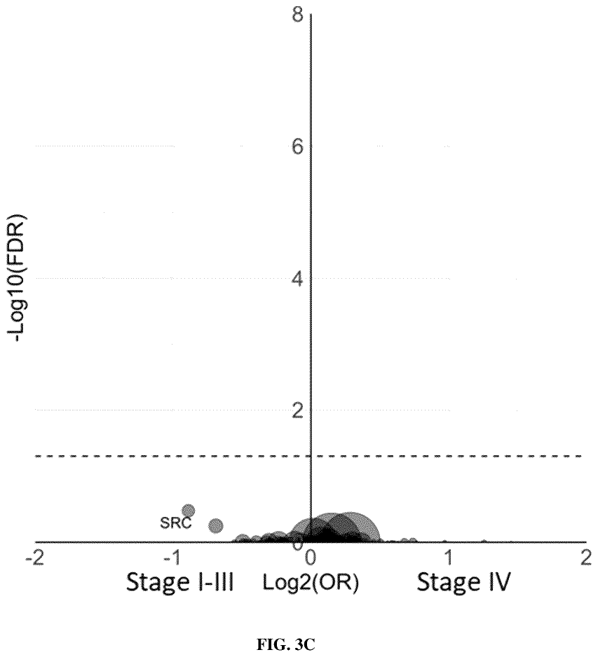

FIGS. 3A-3C show the results of a similar analysis as shown in FIGS. 2A-2D, except that all of the results in FIGS. 3A-3C are in MSS and non-POLE/POLD1 mutated CRC patients. No significant differences were found between actionable alterations in Stage I-III versus Stage IV CRC (FIGS. 3A and 3C), and the KRAS mutation profile was similar between Stage I-III and Stage IV CRC (FIG. 3B).

Comparison of CRC Patients with Early Versus Late CRC Recurrence

To investigate if certain biomarkers were associated with CRC recurrence, the study focused further on Stage I-III CRC patients with specimen collections occurring ≤3 months after the diagnosis date, and who had a known CRC recurrence date documented (FIG. 1). Among those patients, 515 patients that recurred early (within ≤1 year from initial diagnosis) and 720 patients that recurred late (>1 year from initial diagnosis) were included in the study. Table 3 provides characteristics of early- and late-recurring patients. Patients with early recurrence were older at initial diagnosis, with a median age of 63 years, as compared to late-recurring patients with a median age of 61 years (p-value=0.038). Patients with early recurrence also had higher prevalence of MSI-H tumors (p-value=1.52e-7) and TMB ³10 mutations per Mb (p value=3.57e-8).

TABLE 3
Characteristics of patients with early CRC recurrence
(≤1 year from initial diagnosis) and late CRC
recurrence (>1 year from initial diagnosis).

| | Early recurrence | | Late recurrence | | |
|---|---|---|---|---|---|
| | (N = 515) | | (N = 720) | | p-value |
| Age at diagnosis, | 63.0 | (53.0, 73.0) | 61.0 | (53.0, 70.0) | 0.038 |
| Median (IQR) | | | | | |
| Gender (M) | 271 | (52.6%) | 407 | (56.5%) | 0.174 |
| Predicted ancestry | | | | | 0.585 |
| AFR | 65 | (12.6%) | 82 | (11.4%) | |
| AMR | 36 | (7.0%) | 65 | (9.0%) | |
| EAS | 19 | (3.7%) | 30 | (4.2%) | |
| EUR | 391 | (75.9%) | 534 | (74.2%) | |
| SAS | 4 | (0.8%) | 9 | (1.2%) | |
| Biopsy site | | | | | 0.0713 |
| Colon | 437 | (84.9%) | 576 | (80.0%) | |
| Colorectal NOS | 1 | (0.2%) | 4 | (0.6%) | |
| Rectum | 77 | (15.0%) | 140 | (19.4%) | |
| Stage at diagnosis | | | | | 0.020 |
| I | 12 | (2.3%) | 39 | (5.4%) | |
| II | 119 | (23.1%) | 174 | (24.2%) | |
| III | 384 | (74.6%) | 507 | (70.4%) | |
| PD-L1 positive | 24 | (16.1%) | 21 | (12.2%) | 0.316 |
| (DAKO 22C3), | | | | | |
| TPS ≥1 | | | | | |
| TMB | 3.8 | (2.5, 6.3) | 3.5 | (1.7, 5.0) | 5.05e−05 |
| TMB ≥10 | 83 | (16.1%) | 46 | (6.4%) | 3.57e−08 |
| MSI-H | 68 | (13.2%) | 30 | (4.2%) | 1.52e−07 |

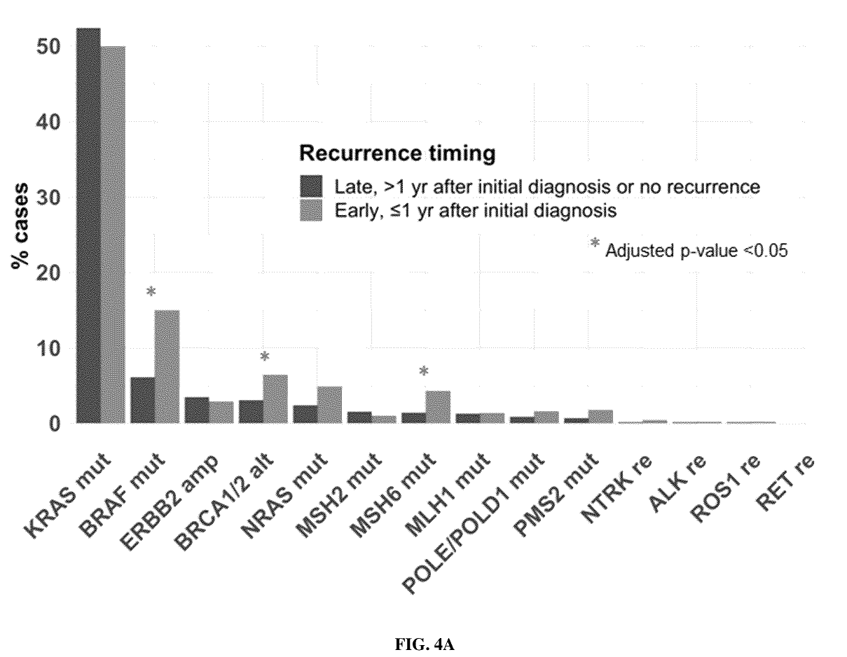

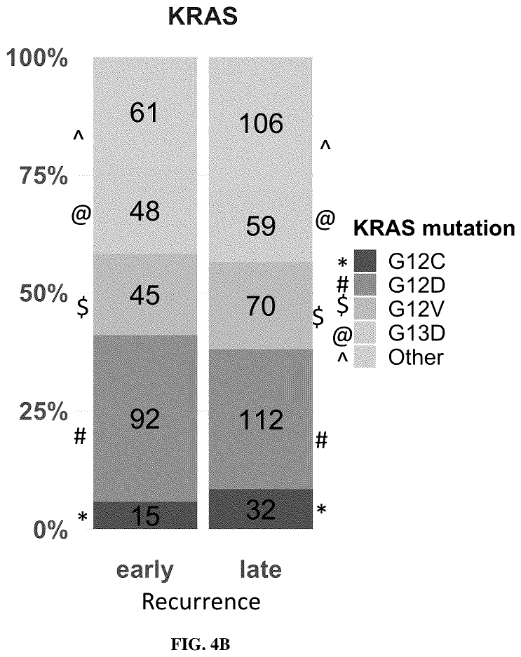

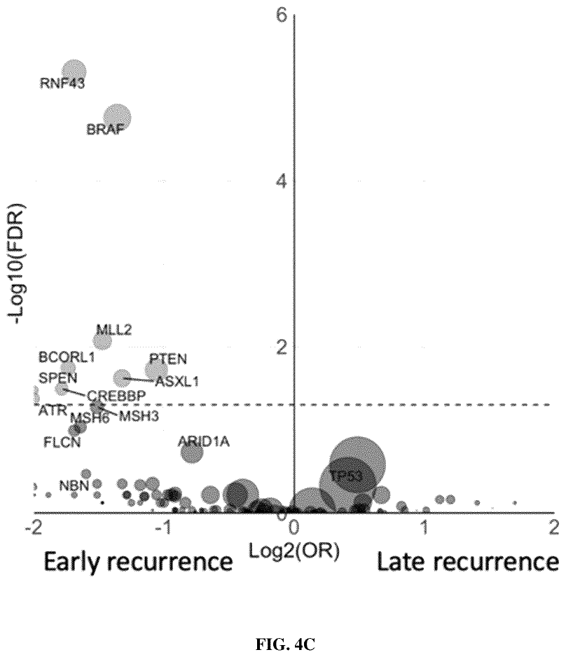

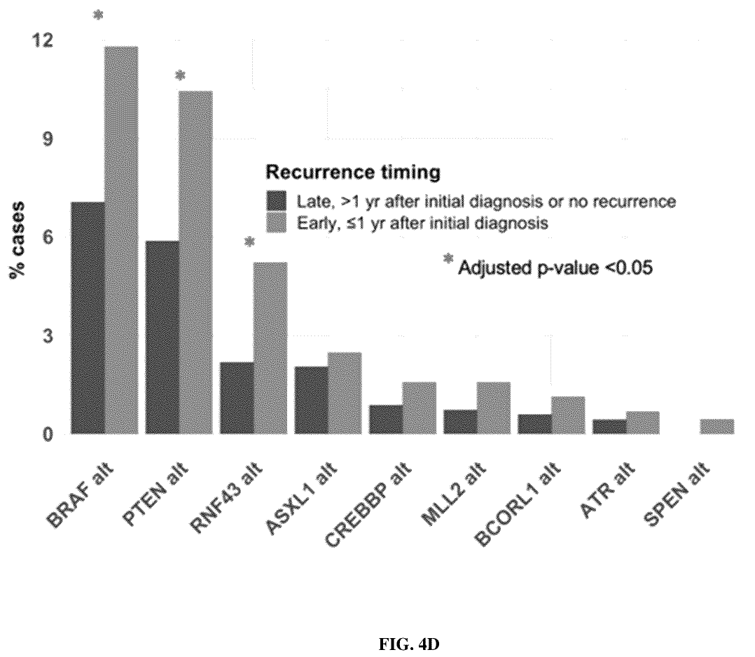

FIG. 4A shows the prevalence of mutations in the indicated genes in early- and late-recurring CRC patients. Mutations in BRAF, BRCA1/2, and MSH6 were enriched in early-recurring CRC patients. KRAS was the most frequently mutated gene for both subgroups. FIG. 4B shows the prevalence of certain specific KRAS mutations identified in early- and late-recurring CRC patients. A further analysis of gene alterations in early- and late-recurring CRC patients showed that mutations in BRAF, PTEN, RNF43, ASXL1, CREBBP, MLL2, BCORL1, ATR, and SPEN were enriched in early-recurring CRC patients (see the volcano plot in FIG. 4C). Of the gene alterations identified in FIG. 4C, alterations in BRAF, PTEN, and RNF43 were observed to be significantly enriched in early-recurring patients that were MSS and did not have POLE/POLD1 mutations (adjusted p-value<0.05) (FIG. 4D).

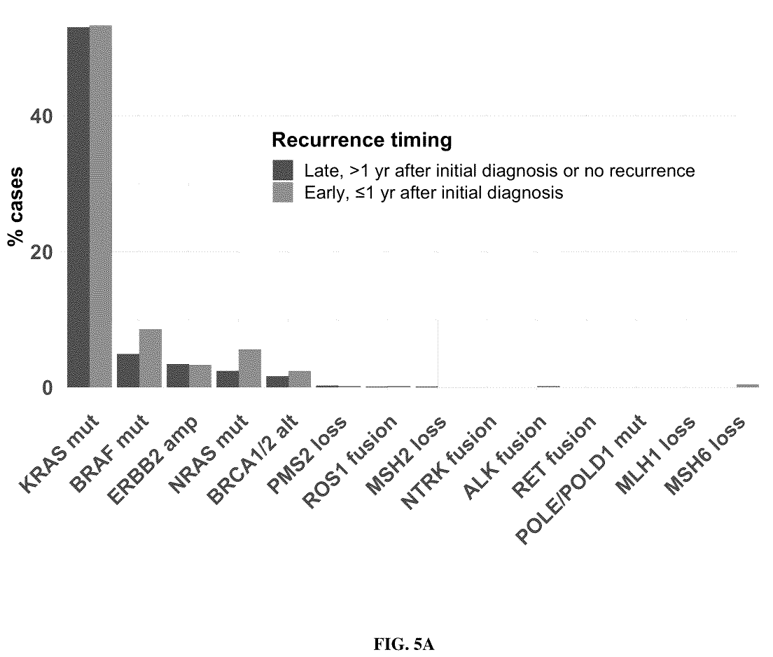

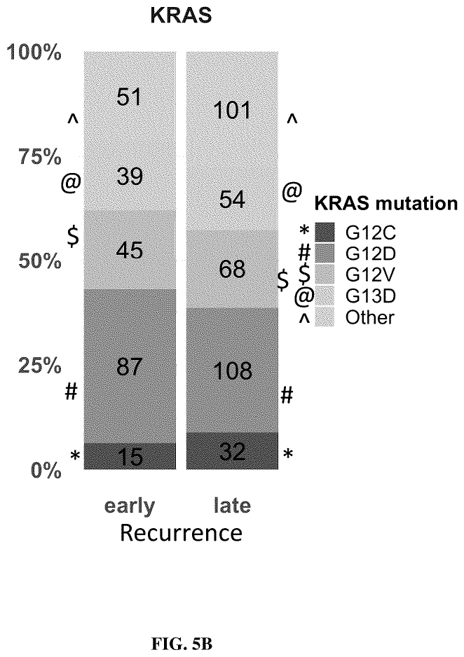

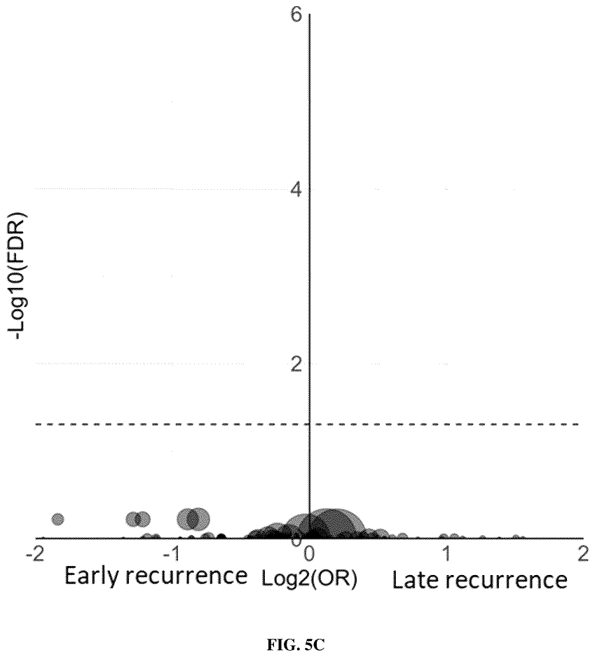

A similar analysis was carried out as above, except in MSS and non-POLE/POLD1 mutated CRC patients. FIGS. 5A and 5C show genomic alterations that were identified in early- and late-recurring CRC patients. FIG. 5B shows the prevalence of certain specific KRAS mutations in early- and late-recurring CRC patients.

### CONCLUSIONS

The results described in this Example demonstrate that alterations in BRAF, BRCA1/2, MSH6, PTEN, RNF43, ASXL1, CREBBP, MLL2, BCORL1, ATR, and SPEN were associated with early recurrence of CRC. Of those, BRAF, PTEN and RNF43 were associated with early recurrence regardless of MSI status. In addition, CRC patients with early recurrence had a higher prevalence of tumors with MSI-H, TMB≥10 mut/Mb, and a PD-L1 TPS≥1. These biomarkers can be used independently or in combination to identify CRC patients with higher risk of disease recurrence and to guide treatment.

It should be understood from the foregoing that, while particular implementations of the disclosed methods have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of treating or delaying progression of a CRC, comprising:
(a) detecting one or more biomarkers in one or more samples from an individual having a CRC, wherein the one or more biomarkers comprise an MSI-H status, wherein responsive to detecting the one or more biomarkers in the one or more samples, the individual is identified as being at risk for CRC recurrence; and
(b) responsive to detecting the one or more biomarkers in the one or more samples, administering to the individual an effective amount of a treatment that comprises an anti-cancer therapy.

2. A method of treating or delaying progression of a CRC, comprising:
(a) detecting two or more biomarkers in one or more samples from an individual having a CRC, wherein the two or more biomarkers comprise: (i) an MSI-H status and one or more of:
(ii) a high TMB, and
(iii) a PD-L1 positive status,
wherein responsive to detecting the one or more biomarkers in the one or more samples, the individual is identified as being at risk for CRC recurrence; and
(b) responsive to detecting the one or more biomarkers in the one or more samples, administering to the individual an effective amount of a treatment that comprises an anti-cancer therapy.

3. The method of claim 1, wherein the one or more biomarkers further comprise one or more of: a high TMB; a PD-L1 positive status; and an alteration in one or more genes, wherein the one or more genes comprise BRAF, PTEN, RNF43, ASXL1, CREBBP, MLL2, BCORL1, ATR, SPEN, BRCA1, BRCA2, or MSH6, or any combination thereof.

4. The method of claim 3, wherein the one or more biomarkers comprise an alteration in one or more genes, wherein the one or more genes comprise BRAF, PTEN, RNF43, ASXL1, CREBBP, MLL2, BCORL1, ATR, or SPEN, or any combination thereof.

5. The method of claim 1, wherein the CRC is a Stage IV CRC.

6. The method of claim 5, wherein the CRC comprises an alteration in an APC and/or TP53 gene; optionally wherein:
(a) the alteration in an APC gene comprises one or more of: R1450*, R876*, splice site 835−8A>G, T1556fs*3, E1309fs*4, R213*, R216*, R564*, R283*, R232*, R1114*, S1465fs*3, Q1367*, R805*, R499*, Q1429*, Q1406*, E1379*, Q1338*, E1309*, E1306*, Y935*, R554*, E941*, Q1378*, V1414fs*1, R302*, E1353*, Q1291*, Y935fs*1, S1356*, E1322*, E1408*, Q1303*, E1397*, P1319fs*2, E1464fs*8, E1295*, E1286*, L1488fs*19, Q1294*, N1455fs*18, S1411fs*4, K534*, G1312*, S1495fs*12, S1344*, R1399fs*9, F1491fs*16, S1415fs*4, S1400*, S1346*, S1315*, Q1328*, E1345*, E1317*, T1493fs*14, S1436fs*37, S1421fs*52, K1182*, D1486fs*21, R332*, F1396fs*19, A1492fs*22, splice site 1548+1G>A, S457*, S1545*, S1465fs*9, R640W, S943*, S1501fs*6, Q188*, Q1477*, Q1244*, P1442fs*31, P1440fs*33, P1439fs*34, K670*, I1580fs*70, E763*, E1554*, W699*, W423*, V1452fs*21, S1415fs*8, S1355fs*19, Q767*, Q236*, L1302fs*3, E893*, E1573*, E1544*, Y1376*, T1556fs*9, T1438fs*35, Q1480*, Q1096*, P1443fs*30, P1424fs*49, M1383fs*3, L1488fs*26, H1490fs*24, E225*, E1538fs*5, E1374*, E1353fs*62, E1309fs*6, C1387*, Y1376fs*9, W1049*, S932*, S811*, S770*, S1539*, S1400fs*1, S1389fs*5, S1355fs*60, S1327*, S1200fs*7, R653K, R1314fs*7, Q901*, Q793*, Q695*, Q264*, Q1228*, Q1065*, P1433fs*40, L1489fs*19, K1310fs*11, I1580fs*69, I1311fs*4, G1288*, F814fs*6, E991*, E403fs*51, E1577fs*73, E1552*, E1536*, E1530*, E1461*, E1353fs*21, E1284*, E1155*, D170fs*4, C1410*, A1351fs*3, Y935fs*19, Y796fs*2, Y1376fs*10, Y1075*, W685*, V452fs*7, T1445fs*28, splice site 835−1G>A, splice site 645+1G>A, splice site 1409−5A>G, splice site 1409−2A>G, splice site 1312+1G>A, S596*, S1581fs*69, S1567*, S1495fs*19, S1355fs*20, S1298fs*7, S1282*, S1272*, R904fs*7, R904fs*12, R405*, R2237*, Q886*, Q789*, Q757*, Q445*, Q260*, Q1477fs*30, Q1469*, Q1444*, or Q1256*; and/or
(b) the alteration in a TP53 gene comprises one or more of: R175H, R282W, R273H, R248Q, R273C, G245S, R213*, R196*, R248W, R306*, R342*, C176F, M237L, C238Y, P152L, Y220C, G266E, C141Y, splice site 375G>A, V173L, V173M, C135F, R158H, W146*, C176Y, I195T, C275Y, G244S, H179R, H179Y, H214R, R249S, R337C, T211I, V172F, V272M, E285K, E286K, P151S, P278S, R273L, splice site 673−1G>A, T125M, V272L, C275F, E294*, F113V, G244D, G245D, K132N, P27fs*17, Q104*,
splice site 672+1G>T, splice site 782+1G>A, V122fs*26, Y163C, A161T, C135Y, C242F, G266R, L257P, L257Q, N131fs*27, P151H, P153fs*28, S166*, S215G, S215N, splice site 375+1G>T, splice site 559+1G>A, V216M, V73fs*76, C238F, C242fs*5, C242Y, E171*, E204*, E258G, E271K, G244A, G266*, H178fs*3, H193Y, K132R, L194F, N239fs*9, P250L, P278T, Q165*, R110L, R209fs*6, R213L, R213Q, S127F, S215I, splice site 672G>A, splice site 782+1G>T,
splice site 88_96+1delAACGTTCTGG, V274F, Y126C, Y126N, Y205D, Y234C, Y236C, A159V, A276G, C135R, C135W, C141R, C141W, C277F, D259Y, E198*, E258*, E271*, E298*, E51*, F134L, F270C, G244C, G266V, H193L, L130F, L130V, L194P, L35fs*9, MIT, N247I, P191del, Q167*, Q192*, Q317*, R181H, R181P, R249M, S214W, S241fs*6, S261fs*85, S90fs*33, S90fs*59, splice site 375+5G>T,
splice site 375G>C, splice site 376−1G>A, splice site 560−1G>A, splice site 560−1G>T, splice site 560−2A>T, splice site 560−3T>G, splice site 673−1G>T, splice site 920−2A>G, splice site 993+1G>A, V157F, V216L, V274A, V274G, W91*, Y163H, Y205H, Y234H, Y236fs*14, Y236H, C124fs*25, C141*, C182fs*65, C229*, C238R, D393fs*78, E180*, E198fs*49, E221*, E224D, E258A, E258K, E285*, E336fs*4, E339*, E349*, F109V, F134C, F134V, or F270L.

7. The method of claim 1, wherein the CRC is Stage I, Stage II, or Stage III CRC.

8. The method of claim 7, wherein the CRC comprises an alteration in one or more of a BRCA1, BRCA2, MSH6, MLH1, and/or MSH2 gene, optionally wherein:
(a) the alteration in a BRCA1 gene comprises one or more of: K339fs*2, K654fs*47, Q1756fs*74, Y655fs*18, A224fs*4, E181*, E577*, E732*, H1686R, K1711fs*3, L1098fs*4, L63F, Q74*, R1203*, R1495M, R1751*, S1457*, S324fs*16, splice site 4185+2_4185+22>A, splice site 442−2A>G, splice site 5277+1G>A, or V340fs*6;
(b) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*;
(c) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H;
(d) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G; and/or
(e) the alteration in a MSH2 gene comprises one or more of: A230fs*16, S233fs*13, R680*, E580*, E480*, Y408*, splice site 943-1G>C, splice site 942+3A>T, R406*, R389*, Q61*, Q574*, Q324*, L634*, L277fs*5, L187R, K449fs*5, I134fs*8, G683R, E86fs*4, E850*, E188*, C778fs*35, or C778fs*34.

9. The method of claim 7, wherein the CRC comprises an alteration in one or more of a RNF43, MLL2, MSH3, PTCH1, CDK12, ARID1A, ASXL1, MSH6, BCORL1, CTNNB1, MLH1, CIC, MAP3K1, ATR, MSH2, CTCF, JAK1, QKI, CDH1, CASP8, NOTCH3, EP300, BRCA2, MEN1, or BCOR gene, or any combination thereof, optionally wherein:
(a) the alteration in a RNF43 gene comprises one or more of: G659fs*41, R117fs*41, R225fs*194, R117fs*8, R145*, P660fs*41, P660fs*87, R371*, R132*, R330*, R337*, A273fs*147, E37fs*11, K181fs*4, S216L, V479fs*25, Y248*, Y332*, A169T, A193fs*6, A78T, C290*, E258fs*162, E318*, E37*, F103fs*20, G257fs*162, G29*, G659fs*87, H352fs*87, I48T, K60fs*2, L311fs*108, L311fs*132, L53fs*1, L82*, L88fs*13, M1I, M55fs*7, N167fs*1, P370fs*49, P715fs*15, P77fs*18, Q153*, Q233*, Q254*, Q283*, Q426*, Q426fs*77, Q6fs*29, Q8*, R113*, R225fs*195, R286W, R49fs*3, S607L, S687fs*13, splice site 375+1G>A, splice site 583−177_592del187, splice site 687+1G>A, splice site 688−1G>A, splice site 850−2A>G, splice site 952+2T>C, T158fs*10, V271fs*11, V271fs*149, V299fs*120, V490fs*12, W13*, W13fs*26, W159*, W165*, W200*, W302*, Y332fs*110, or Y332fs*11;
(b) the alteration in a MLL2 gene comprises one or more of: P2354fs*30, G1235fs*95, P647fs*283, P648fs*2, R4904*, A1390fs*27, Q836fs*94, A2119fs*25, C2436fs*49, C346fs*17, G5182fs*61, H1497fs*30, R2443fs*6, R4238C, R845fs*3, T382fs*20, T4629fs*11, V1244fs*86, V4799M, A1390fs*42, A2169T, A2205fs*59, A221fs*40, A3552fs*4, C5123*, C5142fs*5, D2769N, E2962fs*42, F1790fs*12, F2494fs*49, F2566fs*17, G1317*, G1960fs*87, G1995*, G2262fs*37, G2265fs*21, G3189*, G3698fs*51, H77fs*53, I4491fs*1, I977fs*23, K1686fs*36, K304fs*30, K3140fs*2, K4843fs*15, L1020fs*36, L1271fs*15, L2331fs*46, L2594fs*97, L3716fs*296, L3880fs*131, L5183fs*16, L5318fs*14, P1460fs*46, P2206fs*58, P2382fs*2, P367fs*35, P4380fs*4, P444fs*2, P4968fs*27, P506fs*424, P583fs*347, P62fs*9, P886fs*44, Q1377R, Q1557*, Q211*, Q3471*, Q3811fs*201, Q3839fs*42, Q3909fs*103, Q3934*, Q3950R, Q4235fs*98, Q4284*, Q791fs*139, Q809fs*121, Q809fs*3, R1252*, R1687fs*4, R2099*, R2471*, R2771*, R2830*, R4198*, R466C, R5048H, R5086*, R5120C, R5282*, R5454*, R755fs*3, S102fs*28, S1107fs*12, S1684fs*38, S1684T, S2532fs*11, S2910fs*32, S3159fs*16, S4010fs*12, S4507fs*12, S456*, S4789fs*27, splice site 14644−1G>T, splice site 16413−2A>G, T209fs*11, T2191fs*11, T698fs*232, V1670fs*52, V3089fs*30, Y2199fs*65, or Y2907fs*3;
(c) the alteration in a MSH3 gene comprises one or more of: K383fs*32, L564fs*1, E342*, K902fs*5, K99fs*3, N1020fs*17, N385fs*19, N524fs*3, N739fs*8, N861fs*6, or R268*;
(d) the alteration in a PTCH1 gene comprises one or more of: S1203fs*52, R1308fs*64, Y1316fs*56, L39fs*41, R1308fs*17, E61fs*18, N97fs*43, V1164I, A563V, C1398fs*54, C727fs*19, E1242K, G526*, L50fs*39, N97fs*20, P643fs*11, R602*, or R6fs*1;
(e) the alteration in a CDK12 gene comprises one or more of: G1461fs*38, Q1291fs*3, T1463fs*50, G1271fs*23, R983*, E59fs*33, E751*, E887*, H1035fs*7, I873fs*11, K445*, L342fs*8, L996*, N474fs*8, N864fs*2, P683fs*70, P686fs*13, P974L, Q115*, Q1418*, R1048*, R1331*, R298*, R890H, S133fs*24, splice site 1047−2A>C, T1346fs*7, or T212fs*18;
(f) the alteration in a ARID1A gene comprises one or more of: D1850fs*33, D1850fs*4, F2141fs*59, G276fs*87, P1326fs*155, P224fs*8, Q766fs*67, K1072fs*21, Q1452fs*29, Q372fs*19, Q758fs*75, Q802fs*15, R1989*, A339fs*24, M1634fs*14, Q372fs*28, R693*, Y551fs*72, G314fs*49, P1115fs*46, P1568fs*44, P1898fs*25, Q1200*, Q1519fs*8, Q1631*, R1501fs*4, S1000Y, S11fs*91, W1073fs*32, A134fs*98, A1539fs*27, A27fs*24, A339fs*61, A62fs*39, A77fs*24, D1850fs*34, D2178fs*47, D2260fs*5, E1297*, E1733*, E1783fs*6, E2058*, E2120*, E992*, G1110fs*51, G122fs*278, G126fs*274, G1740*, G2069fs*50, G2087R, G236fs*163, G277fs*86, G285fs*78, G37fs*14, G801fs*32, G82fs*19, G83fs*28, G987fs*50, K1094fs*67, K1905fs*18, K250*, L1049fs*55, L1841fs*2, L2082fs*53, L2238fs*30, L2270fs*8, M1154fs*7, M1273fs*10, M1318fs*163, M1388fs*94, M1564fs*8, M1595fs*19, M1634fs*1, M890fs*46, N1313fs*168, N2109fs*26, P1175fs*5, P1451fs*41, P1468fs*13, P146fs*86, P1560fs*5, P225fs*175, P469fs*150, Q1188*, Q1212*, Q1250*, Q1327*, Q1327fs*11, Q1420*, Q1512*, Q1584*, Q1650*, Q1708*, Q1835*, Q1835fs*1, Q1974*, Q2115*, Q2176*, Q2176fs*48, Q288*, Q505fs*117, Q521*, Q538*, Q546fs*73, Q566*, Q575fs*46, Q581*, Q611*, Q633*, Q806fs*11, R1223C, R1335*, R1446*, R1461*, R1658fs*40, R1658W, R1722*, R1869fs*30, S1465fs*25, S1645fs*46, S2264*, S255fs*145, S366fs*25, S536fs*87, S617fs*2, S617fs*6, splice site 1921−3_1925delTAGGATCT, splice site 2879−2A>G, splice site 3715+1G>C, splice site 4005−2A>T, T1514fs*13, T1743M, T2252fs*27, T286fs*114, T894fs*25, V1561fs*11, V63fs*38, W1545*, W1670*, W2050*, W2091*, Y1101fs*1, Y1377*, Y2076*, Y222*, Y422*, or Y551fs*68;
(g) the alteration in a ASXL1 gene comprises one or more of: G645fs*58, G646fs*12, R693*, E635fs*15, G646fs*58, Q592*, A627fs*8, E41K, E518*, E566*, E676*, E917*, F354L, G1376fs*74, G643fs*15, G643fs*61, G967del, L983fs*8, N1158fs*6, P1377fs*3, P763fs*12, P808fs*10, Q561fs*1, Q588*, Q695*, Q768fs*6, R541fs*162, R596fs*107, R718fs*7, S1335fs*115, S747fs*25, S892fs*16, splice site 140+2T>G, splice site 471+1G>A, T957fs*26, V737fs*10, W1037*, or W583*;
(h) the alteration in a MSH6 gene comprises one or more of: F1088fs*2, F1088fs*5, F1088fs*3, E946*, F1104fs*11, A1236fs*4, A1320fs*5, A780V, C694fs*4, D390fs*21, E1193K, E744fs*12, E760*, G1070fs*9, G864fs*4, I425fs*9, K1140fs*24, K247fs*32, L1356fs*1, N897fs*9, R1068*, R1172fs*4, R240*, R248fs*8, R298*, or R361H;
(i) the alteration in a BCORL1 gene comprises one or more of: P1681fs*20, A1166fs*56, G1682fs*4, A74fs*42, M644fs*4, A858fs*67, Q1001fs*49, R1299*, S803fs*83, A74fs*20, A971fs*4, E1655*, E619*, I389fs*29, K1207N, K1330fs*17, L275fs*143, N1412fs*38, P323fs*95, Q459*, R1196*, R1297*, R1338*, R1420*, R609*, R743fs*13, splice site 4306−2A>G, or W1105*;
(j) the alteration in a CTNNB1 gene comprises one or more of: S45F, T41A, S45P, Q773*, R587*, S33C, T41I, D32N, E334K, G34E, N387K, R449C, S45A, T257L, W25*, W383R, D17_Q78del, D17_T75del, E568*, G69*, I35_G38del, I35S, K19_S37>N, K335fs*10, K335L, M8_V79del, R376H, R515Q, R582Q, R582W, R90*, R95*, S33F, S33T, S37Y, S45del, splice site 14−11_208del206, splice site 14−110_241>AT, splice site 14−126_222del335, splice site 14−181_241+65del474, splice site 14−23_241del251, splice site 14−265_241+45del538, splice site 14−272_241+69del569, splice site 14−294_242−39del684, splice site 14−338_81>TTAC, splice site 14−39_225del251, splice site 14−48_241+12del288, splice site 14−5_97del89, splice site 14−6_241+74del308, splice site 14−69_242−10del488, splice site 14−7_89del83, splice site 14−80_241+22>CAT, splice site 14−91_241+25del344, splice site 1954+1G>A, splice site 1954+1G>T, splice site 1955−1G>A, splice site 2138−2A>C, splice site 241+1G>C, splice site 32_241+94del304, splice site 60_287del428, splice site 65_274del410, splice site 74_241+52del220, splice site 78_241+11del175, splice site 87_242-79del277, splice site 98_241+8>A, V22 S33del, V22 S37del, W25 I35>C, W383C, or W776*;
(k) the alteration in a MLH1 gene comprises one or more of: N168fs*4, R497fs*11, splice site 790+1G>A, F560fs*7, R226*, R226Q, R265C, Y157fs*15, E102D, E34*, E358*, E489*, E512fs*23, E594fs*22, E605*, E671*, E89*, G244V, I691fs*93, K196fs*6, K618del, M1L, N570fs*21, P593fs*23, Q398*, Q537*, Q689*, S184*, S388fs*5, splice site 1038+1G>C, splice site 1559−1delG, splice site 1668−2A>G, splice site 306+2T>G, splice site 453+1G>T, splice site 545+3A>G, splice site 589−2A>G, or splice site 791−2A>G;
(l) the alteration in a CIC gene comprises one or more of: P1597fs*23, P1248fs*54, P509fs*14, P1116fs*45, T1375fs*40, P135fs*70, P1598fs*16, S1117fs*34, S961fs*6, A785fs*139, A900fs*24, D449fs*23, E367*, G136fs*8, G1600fs*14, P1128fs*33, P1336fs*3, P1529fs*91, P404fs*31, P515fs*8, P518fs*5, P574fs*154, P768fs*156, P911fs*13, P98fs*107, Q378*, R1313W, R201W, R353*, S902fs*21, S904fs*27, splice site 583−1G>A, T1541fs*18, T1541fs*79, or T328fs*78;
(m) the alteration in a MAP3K1 gene comprises one or more of: C635*, E126*, E1293fs*3, E788*, G1074fs*8, G608fs*48, H114fs*50, K1160fs*12, L380fs*4, L915*, N1212fs*33, P74fs*3, Q1022*, Q320*, R208*, R288*, R307fs*5, R532*, S101fs*63, splice site 3983−1G>A, splice site 483−1G>A, splice site 633+2T>A, T1145fs*6, T457fs*31, V1045fs*12, or V569I;
(n) the alteration in a ATR gene comprises one or more of: I774fs*5, R1814fs*10, F1091fs*28, F1134fs*6, I774fs*3, E148*, E1699*, E2579*, F2168*, F222fs*11, I1264fs*14, I691fs*5, I774fs*6, K446fs*11, K773fs*3, L1029fs*20, Q195*, R1015Q, R1814fs*8, R2001*, R223fs*1, R224fs*18, R2533*, R2547*, R2598*, S2207fs*15, S825fs*13, splice site 5381−1G>A, W1591*, or Y1844*;
(o) the alteration in a MSH2 gene comprises one or more of: A230fs*16, C778fs*34, E188*, E86fs*4, R680*, S233fs*13, or Y408*;
(p) the alteration in a CTCF gene comprises one or more of: T204fs*26, T204fs*18, R166C, E363fs*5, A137fs*17, A225V, D194fs*28, E112*, E145*, E182fs*9, G32fs*30, H19fs*15, MIT, N259fs*44, P50L, R11W, R275C, splice site 374−1G>T, splice site 854−1G>T, T317fs*91, Y15H, or Y195*;
(q) the alteration in a JAK1 gene comprises one or more of: K860fs*16, P430fs*2, G741D, or K496N;
(r) the alteration in a QKI gene comprises one or more of: K134fs*14, A313V, E42*, G77fs*14, L236fs*54, R319*, splice site 1010−233_*194del444, splice site 142+1G>A, splice site 143−1G>T, or splice site 546+1G>T;
(s) the alteration in a CDH1 gene comprises one or more of: P126fs*89, P127fs*41, R492fs*44, S70fs*13, A241fs*3, A634V, C28*, D257G, D291N, D400G, G169fs*46, L214P, L711V, P372fs*8, Q16*, R335*, R63*, R74*, S111fs*6, S18fs*39, splice site 1137G>A, splice site 1138−1G>A, splice site 1565+1G>A, splice site 1711+2T>C, splice site 688−1G>T, T323fs*33, T340M, or W532*;
(t) the alteration in a CASP8 gene comprises one or more of: R449*, K490fs*73, F373fs*26, I350fs*4, E212*, R452*, A197fs*14, P411L, R194fs*17, R68*, Y252*, A264fs*24, C196Y, D380fs*19, D380fs*2, E195*, E36*, F152fs*18, F296fs*11, G11R, K478fs*10, K478fs*19, L59fs*12, L62P, N475fs*13, R250W, splice site 1355+2T>C, splice site 151+1G>A, V222fs*13, V268fs*8, V492fs*71, or Y8fs*1;
(u) the alteration in a NOTCH3 gene comprises one or more of: A1802fs*8, C1344fs*76, P695fs*165, C43fs*32, G1318fs*245, G2035fs*50, P1317fs*103, T250fs*122, A1020fs*252, A1927T, C720fs*1, C87fs*149, D352fs*2, E1492fs*84, G2035fs*60, G2081fs*4, G707D, K2069fs*16, L2092fs*57, P2033fs*62, P2115fs*10, P42fs*194, R1589Q, R2031fs*54, R6fs*28, S1448fs*115, S157fs*5, splice site 119−156_197+47del282, splice site 1606+1G>A, splice site 5668−1G>T, T1098fs*174, or W1425*;
(v) the alteration in a EP300 gene comprises one or more of: H2324fs*55, M1470fs*26, R1187H, splice site 1282+1G>A, C1385F, D1399N, H2324fs*29, L2303fs*74, L415P, R86*, C1408*, C1738*, E643fs*2, G54*, K1469fs*3, M1339fs*26, N1236fs*41, N419fs*12, Q2282*, Q498*, Q501fs*6, R1055*, R1281*, R1312*, R1627W, R2185*, R2263*, R2330fs*49, R580Q, R648*, S1214Y, S19fs*19, S2271fs*8, splice site 1169−2A>G, splice site 2379+1G>C, splice site 3671+1G>A, splice site 4453−2A>G, T1021fs*3, or Y1467H;
(w) the alteration in a BRCA2 gene comprises one or more of: T3033fs*29, E2981fs*7, I605fs*9, E2981K, N1784fs*3, N1784fs*7, R2034H, K1472fs*6, K1691fs*15, R2842C, R3052Q, T3033fs*11, T3085fs*26, A1237fs*2, C3233fs*15, D252fs*24, D427fs*3, D946fs*14, E1571*, E2144*, E254*, E2981fs*8, E3316fs*2, E340*, E49*, E597*, E764*, E866*, F15fs*10, G2044fs*7, I1851fs*7, I1929fs*34, I332fs*17, I605fs*11, K2674fs*2, K610fs*4, L1466fs*2, L2304*, M2393fs*19, N1287fs*6, N1784fs*2, N2189fs*2, N863fs*11, N986fs*5, Q1429fs*9, Q73*, Q940*, R2318*, R2651fs*6, R2787H, S1442*, S1685*, S1882*, T3085fs*19, V1862fs*1, W1692fs*3, W2830*, or Y1762*;
(x) the alteration in a MEN1 gene comprises one or more of: R521fs*43, E184V, L105fs*13, R457Q, R521fs*15, R532*, or splice site 799−9G>A; and/or
(y) the alteration in a BCOR gene comprises one or more of: P1587fs*53, Q1174fs*8, K1173fs*31, G400fs*42, R810*, Q1272fs*20, R1480*, S158fs*28, S336fs*45, C1329fs*45, D328fs*50, E1025*, E1030fs*48, E1182fs*6, E485fs*42, G154*, G906fs*5, G95fs*16, H1179fs*1, 11290fs*2, K1061fs*52, K1137fs*4, K1271fs*64, K1330*, K839fs*17, L279fs*21, N1425S, N390fs*53, N529fs*28, P602fs*67, P931fs*15, Q1174*, Q1274*, Q348*, Q430*, Q600*, R1053fs*26, R1181fs*1, R1498fs*36, R976*, S1371L, S336fs*42, splice site 166−2A>C, splice site 2997+1G>T, splice site 3239−2A>G, splice site 4072−2A>G, T1331fs*4, or V806fs*10.

10. The method of claim 1, wherein the MSI-H status is detected by sequencing, a polymerase chain reaction (PCR) amplification technique, a non-PCR amplification technique, an isothermal amplification technique, a capillary electrophoresis method, immunohistochemistry, or any combination thereof.

11. The method of claim 3, wherein the one or more biomarkers further comprise a high TMB; optionally wherein the high TMB comprises a TMB of ≥10 mutations/Megabase (mut/Mb) and/or the high TMB is detected by sequencing, whole exome sequencing, whole genome sequencing, gene-targeted sequencing, or next-generation sequencing.

12. The method of claim 3, wherein the one or more biomarkers further comprise a PD-L1 positive status; optionally wherein the PD-L1 positive status is detected based on PD-L1 protein expression as determined using an immunohistochemistry assay.

13. The method of claim 3, wherein:
(a) the alteration in BRAF is a V600E, D594G, G469A, N581S, G466V, K483E, L485F, L485S, or T241M alteration, or any combination thereof;
(b) the alteration in PTEN is a K267fs*9, N323fs*21, R233*, R130*, R130Q, E299*, R173H, T319fs*1, C136Y, C250fs*2, D24fs*20, E157fs*23, E242fs*9, F90fs*9, H93R, I33del, K164fs*3, K330*, L247*, L325P, L57fs*6, N323fs*2, N340fs*3, N63fs*11, P95L, Q171*, Q219*, Q245*, Q261*, R234W, S59*, splice site 209+5G>A, splice site 210−1G>C, splice site 626_634+2delGAACTTGCAGT, splice site 79+1G>A, T131N, or V133I alteration, or any combination thereof;
(c) the alteration in RNF43 is a G659fs*41, R117fs*41, R225fs*194, R132*, R145*, R330*, Y332*, A193fs*6, A273fs*147, A78T, E258fs*162, G257fs*162, K181fs*4, L311fs*132, M1I, P660fs*87, Q153*, Q233*, Q426*, Q426fs*77, Q8*, R225fs*195, R337*, splice site 375+1G>A, V271fs*11, V299fs*120, V479fs*25, W159*, W302*, Y248*, or Y332fs*110V alteration, or any combination thereof;
(d) the alteration in ASXL1 is a G645fs*58, G646fs*12, R693*, A627fs*8, E41K, E676*, G646fs*58, L983fs*8, P1377fs*3, P763fs*12, Q561fs*1, or S892fs*16 alteration, or any combination thereof;
(e) the alteration in CREBBP is a I1084fs*15, P1423fs*36, A1824T, Q1209fs*25, G1145fs*23, R714H, I1084fs*3, K668fs*27, L555fs*7, P2094L, P937fs*61, Q278*, Q911*, R1446H, S801*, splice site 3836+1G>A, or Y1503H alteration, or any combination thereof,
(f) the alteration in MLL2 is a P2354fs*30 9, G1235fs*95, P647fs*283, T382fs*20, A2205fs*59, C2436fs*49, C346fs*17, D2769N, E2962fs*42, F1790fs*12, G2265fs*21, H77fs*53, I977fs*23, K1686fs*36, K304fs*30, L1020fs*36, L5183fs*16, P1460fs*46, P2206fs*58, P367fs*35, P4380fs*4, P444fs*2, P4968fs*27, P506fs*424, P583fs*347, P648fs*2, Q1377R, Q3811fs*201, R1252*, R1687fs*4, R2771*, R2830*, R4238C, R4904*, R5048H, R5282*, R755fs*3, S1107fs*12, S1684T, S2910fs*32, S4507fs*12, S4789fs*27, splice site 14644−1G>T, splice site 16413−2A>G, T209fs*11, V1244fs*86, V1670fs*52, or V4799M alteration, or any combination thereof;
(g) the alteration in BCORL1 is a P1681fs*20, A1166fs*56, A971fs*4, E1655*, E619*, G1682fs*4, K1207N, P323fs*95, Q1001fs*49, R1297*, R1299*, R1420*, or W1105* alteration, or any combination thereof,
(h) the alteration in ATR is a I774fs*5, F1091fs*28, F1134fs*6, I774fs*3, E2579*, F2168*, 1691fs*5, K446fs*11, K773fs*3, R2001*, R223fs*1, R2547*, or W1591* alteration, or any combination thereof,
(i) the alteration in SPEN is a A2105fs*33, R806fs*14, A2105fs*18, H2985fs*199, I1052fs*40, I577fs*37, N2002fs*20, P2495fs*4, P2839fs*50, P3631fs*3, Q253fs*109, R1936*, R2332H, or V1294fs*7 alteration, or any combination thereof,
(j) the alteration in BRCA1 is a K654fs*47, Q1756fs*74, Q74*, R1203*, S324fs*16, splice site 4185+2_4185+22>A, or splice site 442−2A>G alteration, or any combination thereof,
(k) the alteration in BRCA2 is a E2981K, E2981fs*7, N1784fs*7, R2842C, T3033fs*29, A1237fs*2, C3233fs*15, D252fs*24, E2144*, E3316fs*2, E597*, E866*, M2393fs*19, N1287fs*6, N1784fs*3, N2189fs*2, R2034H, R2318*, T3085fs*26, V1862fs*1, or W2830* alteration, or any combination thereof, and/or
(l) the alteration in MSH6 is a F1088fs*5, F1088fs*2, F1088fs*3, E946*, A1236fs*4, A1320fs*5, C694fs*4, K1140fs*24, K247fs*32, L1356fs*1, R240*, R248fs*8, R298*, or R361H alteration, or any combination thereof.

14. The method of claim 3, wherein the alteration in the one or more genes is detected by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing; optionally wherein the sequencing comprises a massively parallel sequencing (MPS) technique, whole genome sequencing (WGS), whole exome sequencing, targeted sequencing, direct sequencing, or a Sanger sequencing technique; and optionally wherein the method further comprises selectively enriching for one or more nucleic acid molecules in a sample from the individual comprising nucleotide sequences corresponding to the one or more genes; wherein the selectively enriching produces an enriched sample.

15. The method of claim 3, further comprising:
(a) optionally, ligating one or more adapters onto nucleic acid molecules in a sample from the individual, thereby generating ligated nucleic acids;
(b) optionally, amplifying nucleic acids from the ligated nucleic acids;
(c) optionally, capturing or enriching from the amplified nucleic acids a plurality of nucleic acids comprising nucleotide sequences corresponding to the one or more genes;
(d) sequencing, by a sequencer, the plurality of nucleic acids to obtain a plurality of sequence reads corresponding to the one or more genes;
(e) analyzing the plurality of sequence reads; and
(f) based on the analysis, detecting the alteration in the one or more genes.

16. The method of claim 1, further comprising:
(a) generating a molecular profile for the individual, based, at least in part, on detecting or acquiring knowledge of the one or more biomarkers in the one or more samples from the individual; and/or
(b) generating a report indicating the presence or absence of the one or more biomarkers in one or more samples from the individual.

17. The method of claim 1, wherein:
(a) detecting the one or more biomarkers in the one or more samples from the individual identifies the individual as one who should be monitored or assessed for CRC recurrence more frequently, as compared to an individual with a CRC that does not comprise the one or more biomarkers;
(b) detecting the one or more biomarkers in the one or more samples from the individual identifies the individual as a candidate to receive a more aggressive anti-cancer therapy for CRC and/or an anti-cancer therapy for CRC of greater duration, as compared to an individual with a CRC that does not comprise the one or more biomarkers; and/or
(c) detecting the presence of the one or more biomarkers in the one or more samples from the individual identifies the individual as one who: (i) should be administered a standard-of-care treatment for CRC, optionally a more aggressive standard-of-care treatment for CRC and/or a standard-of-care treatment for CRC of greater duration, as compared to an individual with a CRC that does not comprise the one or more biomarkers; or (ii) should be administered an anti-cancer therapy other than a standard-of-care treatment for CRC, or an anti-cancer therapy combined with a standard-of-care treatment for CRC; optionally wherein the standard-of-care treatment for CRC comprises a chemotherapy combined with an anti-VEGF agent or an anti-EGFR agent.

18. The method of claim 1, wherein the anti-cancer therapy comprises an immunotherapy.

19. The method of claim 2, wherein the anti-cancer therapy comprises an immunotherapy.

20. The method of claim 1, wherein one or more of the samples from the individual comprise or are derived from a tissue biopsy sample, a liquid biopsy sample, or a normal control.
